SEQUENCE LISTING (33 sequences)

SEQ ID NO: 1 - DNA, 1496 bp, Homo sapiens
  CDS (66)...(389)
cgcccttactcactatagggctcgagcggccgcccgggcaggtgccgcccagtcccgagggcgccatgaggagcctgtgctgcgccccactcctgctcctcttgctgctgccgccgctgctgctcacgccccgcgctggggacgccgccgtgatcaccggggcttgtgacaaggactcccaatgtggtggaggcatgtgctgtgctgtcagtatctgggtcaagagcataaggatttgcacacctatgggcaaactgggagacagctgccatccactgactcgtaaagttccattttttgggcggaggatgcatcacacttgcccatgtctgccaggcttggcctgtttacggacttcatttaaccgatttatttgtttagcccaaaagtaatcgctctggagtagaaaccaaatgtgaatagccacatcttacctgtaaagtcttacttgtgattgtgccaaacaaaaaatgtgccagaaagaaatgctcttgcttcctcaactttccaagtaacatttttatctttgatttgtaaatgatttttttttttttttttatcgaaagagaattttacttttggatagaaatatgaagtgtaaggcattatggaactggttcttatttccctgtttgtgttttggtttgatttggcttttttcttaaatgtcaaaaacgtacccattttcacaaaaatgaggaaaataagaatttgatattttgttagaaaaacttttttttttttttctcaccaccccaagccccatttgtgccctgccgcacaaatacacctacagcttttggtcccttgcctcttccacctcaaagaatttcaaggctcttaccttactttatttttgtccatttctcttccctcctcttgcattttaaagtggagggtttgtctctttgagtttgatggcagaatcactgatgggaatccagctttttgctggcatttaaatagtgaaaagagtgtatatgtgaacttgacactccaaactcctgtcatggcacggaagctaggagtgctgctggacccttcctaaacctgtcactcaagaggacttcagctctgctgttgggctggtgtgtggacagaaggaatggaaagccaaattaatttagtccagatttctaggtttgggtttttctaaaaataaaagattacatttacttcttttactttttataaagttttttttccttagtctcctacttagagatattctagaaaatgtcacttgaagaggaagtatttattttaatctggcacaacactaattaccatttttaaagcggtattaagttgtaatttaaaccttgtttgtaactgaaaggtcgattgtaatggattgccgtttgtacctgtatcagtattgctgtgtaaaaattctgtatcagaataataacagtactgtatatcatttgatttattttaatattatatccttatttttgtcaaaaaaaaaaaaaaaaaaaaaatatgcggccgcg

SEQ ID NO: 2 - protein, 108 aa, Homo sapiens
MRSLCCAPLLLLLLLPPLLLTPRAGDAAVITGACDKDSQCGGGMCCAVSIWVKSIRICTPMGKLGDSCHPLTRKVPFFGRRMHHTCPCLPGLACLRTSFNRFICLAQK

SEQ ID NO: 3 - DNA, 324 bp, Artificial Sequence
  This degenerate sequence encodes the amino acid sequence of SEQ ID NO2
atgmgnwsnytntgytgygcnccnytnytnytnytnytnytnytnccnccnytnytnytnacnccnmgngcnggngaygcngcngtnathacnggngcntgygayaargaywsncartgyggnggnggnatgtgytgygcngtnwsnathtgggtnaarwsnathmgnathtgyacnccnatgggnaarytnggngaywsntgycayccnytnacnmgnaargtnccnttyttyggnmgnmgnatgcaycayacntgyccntgyytnccnggnytngcntgyytnmgnacnwsnttyaaymgnttyathtgyytngcncaraar

SEQ ID NO: 4 - DNA, 1409 bp, Homo sapiens
  CDS (91)...(405)
tggcctccccagcttgccaggcacaaggctgagcgggaggaagcgagaggcatctaagcaggcagtgttttgccttcaccccaagtgaccatgagaggtgccacgcgagtctcaatcatgctcctcctagtaactgtgtctgactgtgctgtgatcacaggggcctgtgagcgggatgtccagtgtggggcaggcacctgctgtgccatcagcctgtggcttcgagggctgcggatgtgcaccccgctggggcgggaaggcgaggagtgccaccccggcagccacaaggtccccttcttcaggaaacgcaagcaccacacctgtccttgcttgcccaacctgctgtgctccaggttcccggacggcaggtaccgctgctccatggacttgaagaacatcaatttttaggcgcttgcctggtctcaggatacccaccatccttttcctgagcacagcctggatttttatttctgccatgaaacccagctcccatgactctcccagtccctacactgactaccctgatctctcttgtctagtacgcacatatgcacacaggcagacatacctcccatcatgacatggtccccaggctggcctgaggatgtcacagcttgaggctgtggtgtgaaaggtggccagcctggttctcttccctgctcaggctgccagagaggtggtaaatggcagaaaggacattccccctcccctccccaggtgacctgctctctttcctgggccctgcccctctccccacatgtatccctcggtctgaattagacattcctgggcacaggctcttgggtgcattgctcagagtcccaggtcctggcctgaccctcaggcccttcacgtgaggtctgtgaggaccaatttgtgggtagttcatcttccctcgattggttaactccttagtttcagaccacagactcaagattggctcttcccagagggcagcagacagtcaccccaaggcaggtgtagggagcccagggaggccaatcagccccctgaagactctggtcccagtcagcctgtggcttgtggcctgtgacctgtgaccttctgccagaattgtcatgcctctgaggccccctcttaccacactttaccagttaaccactgaagcccccaattcccacagcttttccattaaaatgcaaatggtggtggttcaatctaatctgatattgacatattagaaggcaattagggtgtttccttaaacaactcctttccaaggatcagccctgagagcaggttggtgactttgaggagggcagtcctctgtccagattggggtgggagcaagggacagggagcagggcaggggctgaaaggggcactgattcagaccagggaggcaactacacaccaacctgctggctttagaataaaagcaccaactg

SEQ ID NO: 5 - protein, 105 aa, Homo sapiens
MRGATRVSIMLLLVTVSDCAVITGACERDVQCGAGTCCAISLWLRGLRMCTPLGREGEECHPGSHKVPFFRKRKHHTCPCLPNLLCSRFPDGRYRCSMDLKNINF

SEQ ID NO: 6 - DNA, 315 bp, Artificial Sequence
  This degenerate sequence encodes the amino acid sequence of SEQ ID NO5
atgmgnggngcnacnmgngtnwsnathatgytnytnytngtnacngtnwsngaytgygcngtnathacnggngcntgygarmgngaygtncartgyggngcnggnacntgytgygcnathwsnytntggytnmgnggnytnmgnatgtgyacnccnytnggnmgngarggngargartgycayccnggnwsncayaargtnccnttyttymgnaarmgnaarcaycayacntgyccntgyytnccnaayytnytntgywsnmgnttyccngayggnmgntaymgntgywsnatggayytnaaraayathaaytty

SEQ ID NO: 7 - protein, 16 aa, Artificial Sequence
  Peptide linker
GGSGGSGGGGSGGGGS

SEQ ID NO: 8 - protein, 10 aa, Artificial Sequence
  Motif
AVITGACXXD

SEQ ID NO: 9 - protein, 23 aa, Artificial Sequence
  Motif
CHPXXXKVPFFXXRXHHTCPCLP

SEQ ID NO: 10 - protein, 6 aa, Artificial Sequence
  Glu-Glu tag
EYMPME

SEQ ID NO: 11 - DNA, 249 bp, Homo sapiens
atggccgtgatcaccggggcttgtgacaaggactcccaatgtggtggaggcatgtgctgtgctgtcagtatctgggtcaagagcataaggatttgcacacctatgggcaaactgggagacagctgccatccactgactcgtaaagttccattttttgggcggaggatgcatcacacttgcccgtgtctgccaggcttggcctgtttacggacttcatttaaccgatttatttgtttagcccaaaagtaa

SEQ ID NO: 12 - DNA, 68 bp, Artificial Sequence
  oligonucleotide primer ZC40821
ctagaaataattttgtttaactttaagaaggagatatatatatggccgtgatcaccggggcttgtgac

SEQ ID NO: 13 - DNA, 67 bp, Artificial Sequence
  oligonucleotide primer ZC40813
tctgtatcaggctgaaaatcttatctcatccgccaaaacattacttttgggctaaacaaataaatcg

SEQ ID NO: 14 - DNA, 249 bp, Artificial Sequence
  Codon optimized polynucleotide sequence for PROK2
atggctgttattaccggtgcttgcgacaaagactctcagtgtggtggtggtatgtgctgcgctgtttctatctgggttaaatctatccgtatctgcactcctatgggtaaactgggtgactcttgccatccgctgactcgtaaagttccgttcttcggtcgtcgtatgcatcacacctgtccgtgcctgccgggtctggcttgcctgcgtacctctttcaaccgtttcatttgcctggctcagaagtaa

SEQ ID NO: 15 - DNA, 79 bp, Artificial Sequence
  Oligonucleotide primer ZC45,048
agtcaatggatgacaagaatcacccaacttacccataggagtacaaattctgatagacttaacccaaatagaaacagca

SEQ ID NO: 16 - DNA, 77 bp, Artificial Sequence
  Oligonucleotide primer ZC45,049
ttcttgtcatccattgactagaaaggttccattctttggtagaaggatgcatcacacttgtccatgtttgccaggtt

SEQ ID NO: 17 - DNA, 70 bp, Artificial Sequence
  Oligonucleotide primer ZC45,050
ttacttttgagccaaacaaatgaatctgttgaaagaagttctcaaacaagccaaacctggcaaacatgga

SEQ ID NO: 18 - DNA, 68 bp, Artificial Sequence
  Oligonucleotide primer ZC45,051
attactggtgcttgtgataaggattctcaatgtggtggtggtatgtgttgtgctgtttctatttgggt

SEQ ID NO: 19 - DNA, 65 bp, Artificial Sequence
  Oligonucleotide primer ZC45,052
ttatcacaagcaccagtaataacagcagcatcaccggctcttggagtcaacaacaatggtggcaa

SEQ ID NO: 20 - DNA, 59 bp, Artificial Sequence
  Oligonucleotide primer ZC45,053
atgagatctttgtgttgtgctccattgttgttgttgttgttgttgccaccattgttgtt

SEQ ID NO: 21 - DNA, 1182 bp, Homo sapiens
atggagaccaccatggggttcatggatgacaatgccaccaacacttccaccagcttcctttctgtgctcaaccctcatggagcccatgccacttccttcccattcaacttcagctacagcgactatgatatgcctttggatgaagatgaggatgtgaccaattccaggacgttctttgctgccaagattgtcattgggatggccctggtgggcatcatgctggtctgcggcattggaaacttcatctttatcgctgccctggtccgctacaagaaactgcgcaacctcaccaacctgctcatcgccaacctggccatctctgacttcctggtggccattgtctgctgcccctttgagatggactactatgtggtgcgccagctctcctgggagcacggccacgtcctgtgcacctctgtcaactacctgcgcactgtctctctctatgtctccaccaatgccctgctggccatcgccattgacaggtatctggctattgtccatccgctgagaccacggatgaagtgccaaacagccactggcctgattgccttggtgtggacggtgtccatcctgatcgccatcccttccgcctacttcaccaccgagacggtcctcgtcattgtcaagagccaggaaaagatcttctgcggccagatctggcctgtggaccagcagctctactacaagtcctacttcctctttatctttggcatagaattcgtgggccccgtggtcaccatgaccctgtgctatgccaggatctcccgggagctctggttcaaggcggtccctggattccagacagagcagatccgcaagaggctgcgctgccgcaggaagacggtcctggtgctcatgtgcatcctcaccgcctacgtgctatgctgggcgcccttctacggcttcaccatcgtgcgcgacttcttccccaccgtgtttgtgaaggagaagcactacctcactgccttctacatcgtcgagtgcatcgccatgagcaacagcatgatcaacactctgtgcttcgtgaccgtcaagaacgacaccgtcaagtacttcaaaaagatcatgttgctccactggaaggcttcttacaatggcggtaagtccagtgcagacctggacctcaagacaattgggatgcctgccaccgaagaggtggactgcatcagactaaaataa

SEQ ID NO: 22 - DNA, 1155 bp, Homo sapiens
atggcagcccagaatggaaacaccagtttcacacccaactttaatccaccccaagaccatgcctcctccctctcctttaacttcagttatggtgattatgacctccctatggatgaggatgaggacatgaccaagacccggaccttcttcgcagccaagatcgtcattggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccattgccattgacagatatctcgccatcgttcaccccttgaaaccacggatgaattatcaaacggcctccttcctgatcgccttggtctggatggtgtccattctcattgccatcccatcggcttactttgcaacagaaacggtcctctttattgtcaagagccaggagaagatcttctgtggccagatctggcctgtggatcagcagctctactacaagtcctacttcctcttcatctttggtgtcgagttcgtgggccctgtggtcaccatgaccctgtgctatgccaggatctcccgggagctctggttcaaggcagtccctgggttccagacggagcagattcgcaagcggctgcgctgccgcaggaagacggtcctggtgctcatgtgcattctcacggcctatgtgctgtgctgggcacccttctacggtttcaccatcgttcgtgacttcttccccactgtgttcgtgaaggaaaagcactacctcactgccttctacgtggtcgagtgcatcgccatgagcaacagcatgatcaacaccgtgtgcttcgtgacggtcaagaacaacaccatgaagtacttcaagaagatgatgctgctgcactggcgtccctcccagcgggggagcaagtccagtgctgaccttgacctcagaaccaacggggtgcccaccacagaagaggtggactgtatcaggctgaagtga

SEQ ID NO: 23 - DNA, 28 bp, Artificial Sequence
  Oligonucleotide primer ZC29463
ggaattcatgaggagcctgtgctgcgcc

SEQ ID NO: 24 - DNA, 31 bp, Artificial Sequence
  Oligonucleotide primer ZC29464
gctctagacccttttgggctaaacaaataaa

SEQ ID NO: 25 - DNA, 348 bp, Artificial Sequence
  Expression sequence
atgaggagcctgtgctgcgccccactcctgctcctcttgctgctgccgccgctgctgctcacgccccgcgctggggacgccgccgtgatcaccggggcttgtgacaaggactcccaatgtggtggaggcatgtgctgtgctgtcagtatctgggtcaagagcataaggatttgcacacctatgggcaaactgggagacagctgccatccactgactcgtaaagttccattttttgggcggaggatgcatcacacttgcccgtgtctgccaggcttggcctgtttacggacttcatttaaccgatttatttgtttagcccaaaagggtctagaatacatgccgatggac

SEQ ID NO: 26 - protein, 116 aa, Artificial Sequence
  Expression sequence with Gly linker and Glu-Glu-tag
MRSLCCAPLLLLLLLPPLLLTPRAGDAAVITGACDKDSQCGGGMCCAVSIWVKSIRICTPMGKLGDSCHPLTRKVPFFGRRMHHTCPCLPGLACLRTSFNRFICLAQKGLEYMPMD

SEQ ID NO: 27 - protein, 393 aa, Homo sapiens
METTMGFMDDNATNTSTSFLSVLNPHGAHATSFPFNFSYSDYDMPLDEDEDVTNSRTFFAAKIVIGMALVGIMLVCGIGNFIFIAALVRYKKLRNLTNLLIANLAISDFLVAIVCCPFEMDYYVVRQLSWEHGHVLCTSVNYLRTVSLYVSTNALLAIAIDRYLAIVHPLRPRMKCQTATGLIALVWTVSILIAIPSAYFTTETVLVIVKSQEKIFCGQIWPVDQQLYYKSYFLFIFGIEFVGPVVTMTLCYARISRELWFKAVPGFQTEQIRKRLRCRRKTVLVLMCILTAYVLCWAPFYGFTIVRDFFPTVFVKEKHYLTAFYIVECIAMSNSMINTLCFVTVKNDTVKYFKKIMLLHWKASYNGGKSSADLDLKTIGMPATEEVDCIRLK

SEQ ID NO: 28 - protein, 384 aa, Homo sapiens
MAAQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 29 - protein, 129 aa, Homo sapiens
MRSLCCAPLLLLLLLPPLLLTPRAGDAAVITGACDKDSQCGGGMCCAVSIWVKSIRICTPMGKLGDSCHPLTRKNNFGNGRQERRKRKRSKRKKEVPFFGRRMHHTCPCLPGLACLRTSFNRFICLAQK

SEQ ID NO: 30 - DNA, 25 bp, Artificial Sequence
  Oligonucleotide primer ZC41011
ctctccatccttatctttcatcaac

SEQ ID NO: 31 - DNA, 24 bp, Artificial Sequence
  Oligonucleotide primer ZC41012
ctctctgctggctaaacaaaacac

SEQ ID NO: 32 - DNA, 39 bp, Artificial Sequence
  Oligonucleotide primer
gatcgagaattcggccggccaccatgggggacccgcgct

SEQ ID NO: 33 - DNA, 36 bp, Artificial Sequence
  Oligonucleotide primer
tcgatcggatccacgcgttcatttccgggccaagca

(19) United States
(12) Patent Application Publication
(10) Pub. No.: US 2008/0219985 A1
(43) Pub. Date: Sep. 11, 2008

(54) PROK2 ANTAGONISTS AND METHODS OF USE

(76) Inventors: Penny J. Thompson, Snohomish, WA (US); Anthony W. Siadak, Seattle, WA (US); Claire R. Noriega, Shoreline, WA (US); Henry R. Franklin, Seattle, WA (US); Secil Oguz, Bellevue, WA (US); Deborah L. Thompson, Seattle, WA (US); Stavros Topouzis, Seattle, WA (US); Joachim Fruebis, Redmond, WA (US); Kenneth Brasel, Seattle, WA (US); Yue Yao, Issaquah, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 11/531,607

(22) Filed: Sep. 13, 2006

Related U.S. Application Data
(60) Provisional application No. 60/716,586, filed on Sep. 13, 2005.

(51) Int. Cl. A61K 39/395; C07K 16/00; G01N 33/53; A61P 35/00; A61P 9/00

(52) U.S. Cl. 424/139.1; 530/387.9; 436/501

(57) ABSTRACT

The present invention provides methods of using PROK2 and PROK1 antagonist, including monoclonal antibodies to treat inflammation, angiogenesis, and cancer.

### CROSS-REFERENCE TO RELATED APPLICATIONS

This application claims the benefit of U.S. Provisional Application Ser. No. 60/716,586, filed Sep. 13, 2005, which is herein incorporated by reference in its entirety.

### BACKGROUND OF THE INVENTION

Angiogenesis is the sprouting of capillaries from existing blood vessels. During angiogenesis, vascular endothelial cells re-enter the cell cycle, degrade underlying basement membrane, and migrate to form new capillary sprouts. These cells then differentiate, and mature vessels are formed. This process of growth and differentiation is regulated by a balance of pro-angiogenic and anti-angiogenic factors. Angiogenesis occurs during embryonic development, as well as in the adult organism during pregnancy, the female reproductive cycle, and wound healing. In addition, angiogenesis occurs during a variety of pathological conditions, including diabetic retinopathy, macular degeneration, atherosclerosis, psoriasis, rheumatoid arthritis, and solid tumor growth. For review, see Breier et al., Thrombosis and Haemostasis 78:678-683, 1997.

Chief among the angiogenesis-regulating factors are the vascular endothelial growth factors (VEGFs) and the angiopoietins. The VEGFs act through at least three cell surface receptors, designated Flt-1, Flk-1, and Flt-4. The expression of these receptors is limited to certain cell types and/or developmental stages, thereby defining the functions of the ligands. Data obtained from receptor- and growth factor-deficient mice indicate that the VEGFs are essential for vascular development in the embryo. Angiopoietin-1 (Ang-1; see, Davis et al., Cell 87:1161-1169, 1996; and Davis et al., U.S. Pat. No. 5,814,464), acting through the Tie-2 receptor (also known as Tek), is believed to regulate a later stage of vascular development (reviewed by Hanahan, Science 277:48-50, 1997), directing the maturation and stabilization of blood vessels through its action on endothelial cells and the surrounding matrix or mesenchyme. The recently discovered angiopoietin-2 (Ang-2; see, Maisonpierre et al., Science 277:55-60, 1997) is an antagonist of Tie-2-mediated activity. Ang-2 causes a loosening of vessel structure and loss of contact between endothelial cells and the matrix, making the endothelial cells more accessible to VEGF. This destabilization is an initial step in angiogenesis, and both VEGF and Ang-2 are up-regulated at sites of ongoing angiogenesis. Ang-2 is also highly expressed during vascular regression in non-productive ovarian follicles.

In addition to their role in angiogenesis, the angiopoietins may be regulators of hematopoiesis. Endothelial cells and hematopoietic stem cells are believed to be derived from a common precursor cell, and Tie receptors are expressed on both cell types. Tie receptors are expressed in several leukemia cell lines with predominantly megakaryoblastic markers (Batard et al., Blood 87:2212-2220, 1996; Kukk et al., Brit. J. Haematol. 98:195-203, 1997). Analysis of Tie expression in hematopoietic progenitor cells indicates the presence of Tie-mediated pathways in both early hematopoiesis and differentiation and/or proliferation of B cells (Hashiyama et al., Blood 87:93-101, 1996).

The role of growth factors in controlling cellular processes makes them likely candidates and targets for therapeutic intervention. Platelet-derived growth factor, for example, has been disclosed for the treatment of periodontal disease (U.S. Pat. No. 5,124,316) and gastrointestinal ulcers (U.S. Pat. No. 5,234,908). Inhibition of PDGF receptor activity has been shown to reduce intimal hyperplasia in injured baboon arteries (Giese et al., Restenosis Summit VIII, Poster Session #23, 1996; U.S. Pat. No. 5,620,687). Vascular endothelial growth factors have been shown to promote the growth of blood vessels in ischemic limbs (Isner et al., The Lancet 348:370-374, 1996), and have been proposed for use as wound-healing agents, for treatment of periodontal disease, for promoting endothelialization in vascular graft surgery, and for promoting collateral circulation following myocardial infarction (WIPO Publication No. WO 95/24473; U.S. Pat. No. 5,219,739). VEGFs are also useful for promoting the growth of vascular endothelial cells in culture. A soluble VEGF receptor (soluble flt-1) has been found to block binding of VEGF to cell-surface receptors and to inhibit the growth of vascular tissue in vitro (Biotechnology News 16(17):5-6, 1996). Experimental evidence suggests that inhibition of angiogenesis may be used to block tumor development (Biotechnology News, Nov. 13, 1997) and that angiogenesis is an early indicator of cervical cancer (Br. J. Cancer 76:1410-1415, 1997). The hematopoietic cytokine erythropoietin has been developed for the treatment of anemias (e.g., EP 613,683). More recently, thrombopoietin has been shown to stimulate the production of platelets in vivo (Kaushansky et al., Nature 369:568-571, 1994).

In view of the proven clinical utility of angiogenesis regulating factors, there is a need in the art for additional such molecules and antagonists thereof, for use as both therapeutic agents and research tools and reagents.

### SUMMARY OF THE INVENTION

The present invention provides proteins useful for the treatment of PROK2 antagonists in cancer, angiogenesis, tumor growth, and inflammation associated with cancer cells or tissues. Other uses of PROK2 antagonists are described in more detail below.

### DESCRIPTION OF THE INVENTION

### 1. Overview

The present invention is directed to novel uses of previously described proteins, PROK1 and PROK2. See U.S. Pat. No. 6,485,938, U.S. Pat. No. 6,828,425, U.S. Pat. No. 6,756,479, and U.S. patent application Ser. Nos. 10/680,800 and 10/680,755, all of which are herein incorporated by reference. PROK2 and PROK1 are also known as Prokineticin2 and Prokineticin1, respectively. As discussed herein, antagonists PROK1 and PROK1, as well as variants and fragments thereof, can be used to mediate cancer, angiogenesis, tumor growth, and inflammation associated with cancer cells or tissues, as well as regulate gastrointestinal function and gastric emptying. Receptors for PROK2 and PROK1 have been identified as G protein-coupled receptors, GPCR73a and GPCR73b. See Lin, D. et al., J. Biol. Chem. 277: 19276-19280, 2002. The GPCR73a and GPCR73b receptors are also known as PK-R1 and PK-R2.

The present invention provides methods of using antagonists of human PROK polypeptides. A nucleic acid molecule containing a sequence that encodes the PROK2 polypeptide has the nucleotide sequence of SEQ ID NO:1. The encoded polypeptide has the following amino acid sequence: MRSLCCAPLL LLLLLPPLLL TPRAGDAAVI TGACDKDSQC GGGMCCAVSI WVKSIRICTP MGKLGDSCHP LTRKVPFFGR RMHHTCPCLP GLACLRTSFN RFICLAQK (SEQ ID NO:2). Thus, the PROK2 nucleotide sequence described herein encodes a polypeptide of 108 amino acids. The putative signal sequences of PROK2 polypeptide reside at amino acid residues 1 to 20, 1 to 21, and 1 to 22 of SEQ ID NO:2. The mature form of the polypeptide comprises the amino acid sequence from amino acid 28 to 108 as shown in SEQ ID NO:2.

A longer form of the sequence as shown in SEQ ID NO:2 is included in the invention described herein. The longer form has the following amino acid sequence: MRSLCCAPLL LLLLLPPLLL TPRAGDAAVI TGACDKDSQC GGGMCCAVSI WVKSIRICTP MGKLGDSCHP LTRKNNFGNG RQERRKRKRS KRKKEVPFFG RRMHHTCPCL PGLACLRTSF NRFICLAQK (SEQ ID NO:29). The putative signal sequence of the longer form has a mature form that comprises the amino acid sequence from amino acid 28 to 129 as shown in SEQ ID NO:29.

An illustrative nucleic acid molecule containing a sequence that encodes the PROK1 polypeptide has the nucleotide sequence of SEQ ID NO:4. The encoded polypeptide has the following amino acid sequence: MRGATRVSIM LLLVTVSDCA VITGACERDV QCGAGTCCAI SLWLRGLRMC TPLGREGEEC HPGSHKVPFF RKRKHHTCPC LPNLLCSRFP DGRYRCSMDL KNINF (SEQ ID NO:5). Thus, the PROK1 nucleotide sequence described herein encodes a polypeptide of 105 amino acids. The putative signal sequences of PROK1 polypeptide reside at amino acid residues 1 to 17, and 1 to 19 of SEQ ID NO:5.

As described below, the present invention provides isolated polypeptides comprising an amino acid sequence that is at least 70%, at least 75%, at least 80%, at least 85%, at least 90%, or at least 95% identical to amino acid residues 23 to 108 of SEQ ID NO:2, to amino acid residues 28 to 108 of SEQ ID NO:2, or to amino acid residues 28 to 129 if SEQ ID NO:29. Certain of such isolated polypeptides can specifically bind with an antibody that specifically binds with a polypeptide consisting of the amino acid sequence of SEQ ID NO:2. Particular antibodies or antibody fragments can decrease gastric cancer, angiogenesis, tumor growth, and inflammation associated with cancer cells or tissues. An illustrative polypeptide is a polypeptide that comprises the amino acid sequence of SEQ ID NO:2.

Similarly, the present invention provides antibodies or antibody fragments that bind to polypeptides comprising an amino acid sequence that is at least 70%, at least 75%, at least 80%, at least 85%, at least 90%, or at least 95% identical to amino acid residues 20 to 105 of SEQ ID NO:5, wherein such isolated polypeptides can specifically bind with an antibody that specifically binds with a polypeptide consisting of the amino acid sequence of SEQ ID NO:5. An illustrative polypeptide is a polypeptide that comprises the amino acid sequence of SEQ ID NO:5.

The present invention also provides antibodies or antibody fragments that bind to polypeptides comprising an amino acid sequence selected from the group consisting of: (1) amino acid residues 21 to 108 of SEQ ID NO:2, (2) amino acid residues 22 to 108 of SEQ ID NO:2, (3) amino acid residues 23 to 108 of SEQ ID NO:2, (4) amino acid residues 82 to 108 of SEQ ID NO:2, (5) amino acid residues 1 to 78 (amide) of SEQ ID NO:2, (6) amino acid residues 1 to 79 of SEQ ID NO:2, (7) amino acid residues 21 to 78 (amide) of SEQ ID NO:2, (8) amino acid residues 21 to 79 of SEQ ID NO:2, (9) amino acid residues 22 to 78 (amide) of SEQ ID NO:2, (10) amino acid residues 22 to 79 of SEQ ID NO:2, (11) amino acid residues 23 to 78 (amide) of SEQ ID NO:2, (12) amino acid residues 23 to 79 of SEQ ID NO:2, (13) amino acid residues 20 to 108 of SEQ ID NO:2, (14) amino acid residues 20 to 72 of SEQ ID NO:2, (15) amino acid residues 20 to 79 of SEQ ID NO:2, (16) amino acid residues 20 to 79 (amide) of SEQ ID NO:2, (17) amino acid residues 21 to 72 of SEQ ID NO:2, (18) amino acid residues 21 to 79 (amide) of SEQ ID NO:2, (19) amino acid residues 22 to 72 of SEQ ID NO:2, (20) amino acid residues 22 to 79 (amide) of SEQ ID NO:2, (21) amino acid residues 23 to 72 of SEQ ID NO:2, (22) amino acid residues 23 to 79 (amide) of SEQ ID NO:2, (23) amino acid residues 28 to 108 of SEQ ID NO:2, (24) amino acid residues 28 to 72 of SEQ ID NO:2, (25) amino acid residues 28 to 79 of SEQ ID NO:2, (26) amino acid residues 28 to 79 (amide) of SEQ ID NO:2, (27) amino acid residues 75 to 108 of SEQ ID NO:2, (28) amino acid residues 75 to 79 of SEQ ID NO:2, (29) amino acid residues 28 to 108 of SEQ ID NO:2; and (30) amino acid residues 75 to 78 (amide) of SEQ ID NO:2. Illustrative polypeptides consist of amino acid sequences (1) to (30). The present invention also included antibodies polypeptide comprising an amino acid sequence comprising amino acid 28 to 129 as shown in SEQ ID NO:29, and/or fragments thereof.

The present invention further includes antibody or antibody fragments that bind to polypeptides comprising an amino acid sequence selected from the group consisting of: (a) amino acid residues 20 to 105 of SEQ ID NO:5, (b) amino acid residues 18 to 105 of SEQ ID NO:5, (c) amino acid residues 1 to 70 of SEQ ID NO:5, (d) amino acid residues 20 to 70 of SEQ ID NO:5, (e) amino acid residues 18 to 70 of SEQ ID NO:5, (f) amino acid residues 76 to 105 of SEQ ID NO:5, (g) amino acid residues 66 to 105 of SEQ ID NO:5, and (h) amino acid residues 82 to 105 of SEQ ID NO:5. Illustrative polypeptides consist of amino acid sequences (a) to (h).

The present invention further provides antibodies and antibody fragments that specifically bind with such polypeptides. Exemplary antibodies include polyclonal antibodies, murine monoclonal antibodies, humanized antibodies derived from murine monoclonal antibodies, and human monoclonal antibodies. Illustrative antibody fragments include F(ab′)₂, F(ab)₂, Fab′, Fab, Fv, scFv, and minimal recognition units. The present invention also includes anti-idiotype antibodies that specifically bind with such antibodies or antibody fragments. The present invention further includes compositions comprising a carrier and a peptide, polypeptide, antibody, or anti-idiotype antibody described herein.

The present invention also includes vectors and expression vectors comprising nucleic acid molecules encoding PROK antagonists, including antbodies and antibody fragments. Such expression vectors may comprise a transcription promoter, and a transcription terminator, wherein the promoter is operably linked with the nucleic acid molecule, and wherein the nucleic acid molecule is operably linked with the transcription terminator. The present invention further includes recombinant host cells comprising these vectors and expression vectors. Illustrative host cells include bacterial, yeast, avian, fungal, insect, mammalian, and plant cells. Recombinant host cells comprising such expression vectors can be used to prepare PROK polypeptides by culturing such recombinant host cells that comprise the expression vector and that produce the PROK protein, and, optionally, isolating the PROK protein from the cultured recombinant host cells. The present invention further includes products made by such processes.

In addition, the present invention provides pharmaceutical compositions comprising a pharmaceutically acceptable carrier and at least one of such an expression vector or recombinant virus comprising such expression vectors.

The present invention further provides methods for detecting the presence of PROK polypeptide in a biological sample, comprising the steps of: (a) contacting the biological sample with an antibody or an antibody fragment that specifically binds with a polypeptide either consisting of the amino acid sequence of SEQ ID NO:2 or consisting of the amino acid sequence of SEQ ID NO:5, wherein the contacting is performed under conditions that allow the binding of the antibody or antibody fragment to the biological sample, and (b) detecting any of the bound antibody or bound antibody fragment. Such an antibody or antibody fragment may further comprise a detectable label selected from the group consisting of radioisotope, fluorescent label, chemiluminescent label, enzyme label, bioluminescent label, and colloidal gold.

Illustrative biological samples include human tissue, such as an autopsy sample, a biopsy sample, body fluids and digestive components, and the like.

The present invention also provides a kit for detection of PROK protein may comprise a container that comprises an antibody, or an antibody fragment, that specifically binds with a polypeptide consisting of the amino acid sequence of SEQ ID NO:2 or consisting of the amino acid sequence of SEQ ID NO: 29 or consisting of the amino acid sequence of SEQ ID NO:5.

The present invention also contemplates anti-idiotype antibodies, or anti-idiotype antibody fragments, that specifically bind an antibody or antibody fragment that specifically binds a polypeptide consisting of the amino acid sequence of SEQ ID NO:2 or consisting of the amino acid sequence of SEQ ID NO: 29 or the amino acid sequence of SEQ ID NO:5. The invention also contemplates anti-idiotype antibodies, or anti-idiotype antibody fragments, that specifically bind an antibody or antibody fragment that specifically binds a polypeptide consisting of the amino acid sequence of SEQ ID NO:2 or consisting of the amino acid sequence of SEQ ID NO: 29 or the amino acid sequence of SEQ ID NO:5.

The present invention also provides antibodies, including monoclonal antibodies that specifically bind an antibody or antibody fragment that specifically binds a polypeptide consisting of the amino acid sequence of SEQ ID NO:2 or consisting of the amino acid sequence of SEQ ID NO: 29 or the amino acid sequence of SEQ ID NO:5. The invention also contemplates antibodies, including monocloncal antibodies and antibody fragments, that specifically bind an antibody or antibody fragment that specifically binds a polypeptide consisting of the amino acid sequence of SEQ ID NO:2 or consisting of the amino acid sequence of SEQ ID NO: 29 and the amino acid sequence of SEQ ID NO:5.

The present invention also provides fusion proteins comprising a PROK2 antibody or antibody fragment moiety or a PROK1 polypeptide moiety. Such fusion proteins can further comprise an immunoglobulin moiety. A suitable immunoglobulin moiety is an immunoglobulin heavy chain constant region, such as a human F_{c} fragment. The present invention also includes isolated nucleic acid molecules that encode such fusion proteins.

The invention also provides a method of reducing inflammation comprising administering to the mammal a PROK2 or PROK1 antagonist, wherein the inflammation in the intestine is reduced. In an embodiment, the antagonist is an antibody. In another embodiment, the antagonist is selected from: anti-idiotype antibodies; antibody fragments; chimeric antibodies; and humanized antibodies In an embodiment, the antagonist is a receptor, and wherein the receptor binds the amino acid sequence as shown in SEQ ID NO:2, SEQ ID NO:29, or SEQ ID NO:5. In another embodiment the receptor comprises the amino acid sequence as shown in SEQ ID NO:27 or in SEQ ID NO:28. In another embodiment, the antagonist is a portion of a receptor, and wherein that portion of the receptor specifically binds to the amino acid sequence as shown in SEQ ID NO:2, SEQ ID NO:29, or as shown in SEQ ID NO:5. In another embodiment, the inflammation is chronic. In another embodiment, the inflammation is sporadic. In another embodiment, the inflammation is a symptom of irritable bowel syndrome. In another embodiment, the inflammation is a symptom of inflammatory bowel disease. In a further embodiment, the inflammatory bowel disease is ulcerative colitis or Crohn's disease. In another embodiment, the inflammation is associated with cancer. In another embodiment, the inflammation is associated with prognosis of cancer, including tumor progression staging.

The invention also provides a method of treating inflammation comprising administering to the mammal a PROK2 or PROK1 antagonist, wherein the inflammation is reduced. In an embodiment, the antagonist is an antibody. In another embodiment, the antagonist is selected from: anti-idiotype antibodies; antibody fragments; chimeric antibodies; and humanized antibodies. In another embodiment, the antagonist is a receptor, and wherein the receptor binds the amino acid sequence as shown in SEQ ID NO:2, SEQ ID NO:29, or SEQ ID NO:5. In another embodiment, the receptor comprises the amino acid sequence as shown in SEQ ID NO:27 or SEQ ID NO:28. In an embodiment, the antagonist is a portion a receptor, and that portion of the receptor specifically binds to the amino acid sequence as shown in SEQ ID NO:2, SEQ ID NO:29, or as shown in SEQ ID NO:5. In another embodiment, the inflammation is chronic. In another embodiment, the inflammation is sporadic. In another embodiment, the inflammation is a symptom of irritable bowel syndrome. In another embodiment, the inflammation is a symptom inflammatory bowel disease. In a further embodiment, the inflammatory bowel disease is ulcerative colitis, Crohn's disease, or diarrhea-prone irritable bowel syndrome. In another embodiment, the inflammation is associated with cancer. In another embodiment, the inflammation is associated with prognosis of cancer, including tumor progression staging.

The invention also provides a method of detecting inflammatory bowel disease in a biological sample, comprising screening the sample for the polypeptide sequence as shown in SEQ ID NO:2, SEQ ID NO:29, or SEQ ID NO:5 or a fragment thereof.

The invention also provides a method of detecting irritable bowel syndrome, in a biological sample, comprising screening the sample for the polypeptide sequence as shown in SEQ ID NO:2, SEQ ID NO:29, or SEQ ID NO:5 or a fragment thereof.

The invention also provides a method of detecting inflammatory bowel disease in a biological sample, comprising screening the sample for the polynucleotide sequence as shown in SEQ ID NO:1 or SEQ ID NO:4, or a fragment thereof.

The invention also provides a method of diagnosing inflammatory bowel disease in a biological sample, comprising screening the sample for the polypeptide sequence as shown in SEQ ID NO:2, SEQ ID NO:29, or SEQ ID NO:5 or a fragment thereof.

The invention also provides a method of diagnosing irritable bowel syndrome in a biological sample, comprising screening the sample for the polypeptide sequence as shown in SEQ ID NO:2, SEQ ID NO:29, or SEQ ID NO:5 or a fragment thereof.

The invention also provides a method of diagnosing inflammatory bowel disease in a biological sample, comprising screening the sample for the polynucleotide sequence as shown in SEQ ID NO:1 or SEQ ID NO:4, or a fragment thereof.

The invention also provides a method of treating inflammatory bowel disease in a mammal in need thereof, comprising administering to the mammal a polypeptide, wherein the polypeptide comprises the amino acid sequence of amino acid residues 28 to 108 of SEQ ID NO:2, amino acid residues 28 to 129 of SEQ ID NO:29, or amino acid residues 20 to 105 of SEQ ID NO:5.

The invention also provides a method of treating irritable bowel syndrome in a mammal in need thereof, comprising administering to the mammal a polypeptide, wherein the polypeptide comprises the amino acid sequence of amino acid residues 28 to 108 of SEQ ID NO:2, amino acid residues 28 to 129 of SEQ ID NO:29, or amino acid residues 20 to 105 of SEQ ID NO:5.

The invention also provides a method of treating irritable bowel syndrome in a mammal in need thereof, comprising administering to the mammal a polynucleotide, wherein the polynucleotide comprises the nucleic acid sequence of SEQ ID NO:1 or of SEQ ID NO:5.

The invention also provides a method of inhibiting, reducing or delaying progression of cancer comprising administering an antibody, or variant or fragment thereof, to a patient or a patient sample. In an embodiment, the antibody is a monoclonal antibody that specifically binds a polypeptide, wherein the polypeptide comprises the amino acid sequence of amino acid residues 28 to 108 of SEQ ID NO:2, amino acid residues 28 to 129 of SEQ ID NO:29, or amino acid residues 20 to 105 of SEQ ID NO:5. In another embodiment, the antibody is a monoclonal antibody produced by a hybridoma described herein. In another embodiment, the cancer is selected from colon cancer, intestinal cancer, lung cancer, breast cancer, ovarian cancer, and pancreas cancer.

The invention also provides a method of inhibiting, reducing or delaying progression of tumor size comprising administering an antibody, or variant or fragment thereof, to a patient or a patient sample. In an embodiment, the antibody is a monoclonal antibody that specifically binds a polypeptide, wherein the polypeptide comprises the amino acid sequence of amino acid residues 28 to 108 of SEQ ID NO:2, amino acid residues 28 to 129 of SEQ ID NO:29, or amino acid residues 20 to 105 of SEQ ID NO:5. In another embodiment, the antibody is a monoclonal antibody produced by a hybridoma described herein. In another embodiment, the tumor is selected from colon tumor, intestinal tumor, lung tumor, breast tumor, ovarian tumor, and pancreas tumor. In another embodiment, the tumor is a solid organ tumor.

These and other aspects of the invention will become evident upon reference to the following detailed description. In addition, various references are identified below and are incorporated by reference in their entirety.

### 2. Definitions

In the description that follows, a number of terms are used extensively. The following definitions are provided to facilitate understanding of the invention.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “complement of a nucleic acid molecule” refers to a nucleic acid molecule having a complementary nucleotide sequence and reverse orientation as compared to a reference nucleotide sequence.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (i.e., GAU and GAC triplets each encode Asp).

An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

“Linear DNA” denotes non-circular DNA molecules having free 5′ and 3′ ends. Linear DNA can be prepared from closed circular DNA molecules, such as plasmids, by enzymatic digestion or physical disruption.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SPI, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the TATA box and start of transcription. By this definition, a core promoter may or may not have detectable activity in the absence of specific sequences that may enhance the activity or confer tissue specific activity.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner.

An “enhancer” is a type of regulatory element that can increase the efficiency of transcription, regardless of the distance or orientation of the enhancer relative to the start site of transcription.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

A “polypeptide” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A peptide or polypeptide encoded by a non-host DNA molecule is a “heterologous” peptide or polypeptide.

An “integrated genetic element” is a segment of DNA that has been incorporated into a chromosome of a host cell after that element is introduced into the cell through human manipulation. Within the present invention, integrated genetic elements are most commonly derived from linearized plasmids that are introduced into the cells by electroporation or other techniques. Integrated genetic elements are passed from the original host cell to its progeny.

A “cloning vector” is a nucleic acid molecule, such as a plasmid, cosmid, or bacteriophage, that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces a PROK2 or PROK1 peptide or polypeptide from an expression vector. In contrast, such polypeptides can be produced by a cell that is a “natural source” of PROK2 or PROK1, and that lacks an expression vector.

A “fusion protein” is a hybrid protein expressed by a nucleic acid molecule comprising nucleotide sequences of at least two genes. For example, a fusion protein can comprise at least part of a PROK2 or PROK1 polypeptide fused with a polypeptide that binds an affinity matrix. Such a fusion protein provides a means to isolate large quantities of PROK2 or PROK1 using affinity chromatography.

The term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

The term “secretory signal sequence” denotes a DNA sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “expression” refers to the biosynthesis of a gene product. For example, in the case of a structural gene, expression involves transcription of the structural gene into mRNA and the translation of mRNA into one or more polypeptides.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene.

As used herein, the term “immunomodulator” includes cytokines, stem cell growth factors, lymphotoxins, co-stimulatory molecules, hematopoietic factors, and synthetic analogs of these molecules.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of less than 10⁹ M⁻¹.

An “anti-idiotype antibody” is an antibody that binds with the variable region domain of an immunoglobulin. In the present context, an anti-idiotype antibody binds with the variable region of an anti-PROK2 or anti-PROK1 antibody, and thus, an anti-idiotype antibody mimics an epitope of PROK2 or PROK1.

An “antibody fragment” is a portion of an antibody such as F(ab′)₂, F(ab)₂, Fab′, Fab, and the like. Regardless of structure, an antibody fragment binds with the same antigen that is recognized by the intact antibody. For example, an anti-PROK2 monoclonal antibody fragment binds with an epitope of PROK2.

The term “antibody fragment” also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A “chimeric antibody” is a recombinant protein that contains the variable domains and complementary determining regions derived from a rodent antibody, while the remainder of the antibody molecule is derived from a human antibody.

“Humanized antibodies” are recombinant proteins in which murine complementarity determining regions of a monoclonal antibody have been transferred from heavy and light variable chains of the murine immunoglobulin into a human variable domain.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNAs encoding affinity tags are available from commercial suppliers (e.g., Pharmacia Biotech, Piscataway, N.J.).

A “naked antibody” is an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

As used herein, the term “antibody component” includes both an entire antibody and an antibody fragment.

A “target polypeptide” or a “target peptide” is an amino acid sequence that comprises at least one epitope, and that is expressed on a target cell, such as a tumor cell, or a cell that carries an infectious agent antigen. T cells recognize peptide epitopes presented by a major histocompatibility complex molecule to a target polypeptide or target peptide and typically lyse the target cell or recruit other immune cells to the site of the target cell, thereby killing the target cell.

An “antigenic peptide” is a peptide, which will bind a major histocompatibility complex molecule to form an MHC-peptide complex which is recognized by a T cell, thereby inducing a cytotoxic lymphocyte response upon presentation to the T cell. Thus, antigenic peptides are capable of binding to an appropriate major histocompatibility complex molecule and inducing a cytotoxic T cells response, such as cell lysis or specific cytokine release against the target cell which binds or expresses the antigen. The antigenic peptide can be bound in the context of a class I or class II major histocompatibility complex molecule, on an antigen presenting cell or on a target cell.

In eukaryotes, RNA polymerase II catalyzes the transcription of a structural gene to produce mRNA. A nucleic acid molecule can be designed to contain an RNA polymerase II template in which the RNA transcript has a sequence that is complementary to that of a specific mRNA. The RNA transcript is termed an “anti-sense RNA” and a nucleic acid molecule that encodes the anti-sense RNA is termed an “anti-sense gene.” Anti-sense RNA molecules are capable of binding to mRNA molecules, resulting in an inhibition of mRNA translation.

The term “variant PROK2 gene” refers to nucleic acid molecules that encode a polypeptide having an amino acid sequence that is a modification of SEQ ID NO:2. Such variants include naturally-occurring polymorphisms of PROK2 genes, as well as synthetic genes that contain conservative amino acid substitutions of the amino acid sequence of SEQ ID NO:2. Additional variant forms of PROK2 genes are nucleic acid molecules that contain insertions or deletions of the nucleotide sequences described herein. A variant PROK2 gene can be identified by determining whether the gene hybridizes with a nucleic acid molecule having the nucleotide sequence of SEQ ID NO:1, or its complement, under stringent conditions. Similarly, a variant PROK1 gene and a variant PROK1 polypeptide can be identified with reference to SEQ ID NO:4 and SEQ ID NO:5, respectively.

Alternatively, variant PROK genes can be identified by sequence comparison. Two amino acid sequences have “100% amino acid sequence identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art (see, for example, Peruski and Peruski, The Internet and the New Biology: Tools for Genomic and Molecular Research (ASM Press, Inc. 1997), Wu et al. (eds.), “Information Superhighway and Computer Databases of Nucleic Acids and Proteins,” in Methods in Gene Biotechnology, pages 123-151 (CRC Press, Inc. 1997), and Bishop (ed.), Guide to Human Genome Computing, 2nd Edition (Academic Press, Inc. 1998)). Particular methods for determining sequence identity are described below.

Regardless of the particular method used to identify a variant PROK2 gene or variant PROK2 polypeptide, a variant gene or polypeptide encoded by a variant gene may be characterized by its ability to bind specifically to an anti-PROK2 antibody. Similarly, a variant PROK1 gene product or variant PROK1 polypeptide may be characterized by its ability to bind specifically to an anti-PROK1 antibody.

The present invention includes functional fragments of PROK2 and PROK1 genes. Within the context of this invention, a “functional fragment” of a PROK2 (or PROK1) gene refers to a nucleic acid molecule that encodes a portion of a PROK2 (or PROK1) polypeptide, which specifically binds with an anti-PROK2 (anti-PROK1) antibody.

Due to the imprecision of standard analytical methods, molecular weights and lengths of polymers are understood to be approximate values. When such a value is expressed as “about” X or “approximately” X, the stated value of X will be understood to be accurate to ±10%.

Production of Human PROK2 and PROK1 Antibodies

Anti-PROK antibodies, produced as described below, can be used to isolate DNA sequences that encode human PROK genes from cDNA libraries. For example, the antibodies can be used to screen λgt11 expression libraries, or the antibodies can be used for immunoscreening following hybrid selection and translation (see, for example, Ausubel (1995) at pages 6-12 to 6-16; Margolis et al., “Screening λ expression libraries with antibody and protein probes,” in DNA Cloning 2: Expression Systems, 2nd Edition, Glover et al. (eds.), pages 1-14 (Oxford University Press 1995)).

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

A limited number of non-conservative amino acids, amino acids that are not encoded by the genetic code, non-naturally occurring amino acids, and unnatural amino acids may be substituted for amino acid residues in the antibody and antibody fragments.

Amino acid sequence analysis indicates that PROK2 and PROK1 share several motifs. For example, one motif is “AVITGAC[DE][KR]D” (SEQ ID NO:8), wherein acceptable amino acids for a given position are indicated within square brackets. This motif occurs in PROK2 at amino acid residues 28 to 37 of SEQ ID NO:2, and in PROK1 at amino acid residues 20 to 29 of SEQ ID NO:5. Another motif is “CHP[GL][ST][HR]KVPFFX[KR]RXHHTCPCLP” (SEQ ID NO:9), wherein acceptable amino acids for a given position are indicated within square brackets, and “X” can be any amino acid residue. This motif occurs in PROK2 at amino acid residues 68 to 90 in SEQ ID NO:2, and in PROK1 at amino acid residues 60 to 82 of SEQ ID NO:5. The present invention includes antibody and antibody fragments that bind to peptides and polypeptides comprising these motifs.

Sequence analysis also indicated that PROK2 and PROK1 include various conservative amino acid substitutions with respect to each other. Accordingly, particular PROK2 variants can be designed by modifying its sequence to include one or more amino acid substitutions corresponding with the PROK1 sequence, while particular PROK1 variants can be designed by modifying its sequence to include one or more amino acid substitutions corresponding with the PROK2 sequence. Such variants can be constructed using Table 1, which presents exemplary conservative amino acid substitutions found in PROK2 and PROK1. Although PROK2 and PROK1 variants can be designed with any number of amino acid substitutions, certain variants will include at least about X amino acid substitutions, wherein X is selected from the group consisting of 2, 5, 7, 10, 12, 14, 16, 18, and 20.

TABLE 1

| PROK2 | | PROK1 | |
|---|---|---|---|
| Amino acid Position | | Amino acid Position | |
| (SEQ ID NO: 2) | Amino acid | (SEQ ID NO: 5) | Amino acid |
| 4 | Leu | 4 | Ala |
| 7 | Ala | 7 | Val |
| 9 | Leu | 9 | Ile |
| 14 | Leu | 14 | Val |
| 35 | Asp | 27 | Glu |
| 36 | Lys | 28 | Arg |
| 42 | Gly | 34 | Ala |
| 48 | Val | 40 | Ile |
| 50 | Ile | 42 | Leu |
| 52 | Val | 44 | Leu |
| 53 | Lys | 45 | Arg |
| 55 | Ile | 47 | Leu |
| 63 | Lys | 55 | Arg |
| 66 | Asp | 58 | Glu |
| 71 | Leu | 63 | Gly |
| 72 | Thr | 64 | Ser |
| 73 | Arg | 65 | His |
| 80 | Arg | 72 | Lys |
| 93 | Ala | 85 | Leu |
| 102 | Phe | 94 | Tyr |

The present invention also antibodies and antibody fragments that bind to “functional fragments” of PROK2 or PROK1 polypeptides and nucleic acid molecules encoding such functional fragments. Routine deletion analyses of nucleic acid molecules can be performed to obtain functional fragments of a nucleic acid molecule that encodes a PROK2 or PROK1 polypeptide. As an illustration, DNA molecules having the nucleotide sequence of SEQ ID NO:1 can be digested with Bal31 nuclease to obtain a series of nested deletions. The fragments are then inserted into expression vectors in proper reading frame, and the expressed polypeptides are isolated and tested for the ability to bind anti-PROK antibodies. One alternative to exonuclease digestion is to use oligonucleotide-directed mutagenesis to introduce deletions or stop codons to specify production of a desired fragment. Alternatively, particular fragments of a PROK gene can be synthesized using the polymerase chain reaction.

The present invention also contemplates functional fragments of a PROK2 or PROK1 gene that have amino acid changes, compared with the amino acid sequence of SEQ ID NO:2 or SEQ ID NO:5. A variant PROK gene can be identified on the basis of structure by determining the level of identity with the particular nucleotide and amino acid sequences disclosed herein. An alternative approach to identifying a variant gene on the basis of structure is to determine whether a nucleic acid molecule encoding a potential variant PROK2 or PROK1 gene can hybridize to a nucleic acid molecule having the nucleotide sequence of SEQ ID NO:1 or SEQ ID NO:4, as discussed above.

The present invention also provides polypeptide fragments or peptides comprising an epitope-bearing portion of a PROK2 or PROK1 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides and polypeptides of the present invention are useful to raise antibodies that bind with the polypeptides described herein.

Antigenic epitope-bearing peptides and polypeptides can contain at least four to ten amino acids, at least ten to fifteen amino acids, or about 15 to about 30 amino acids of SEQ ID NOs:2 or 5. Such epitope-bearing peptides and polypeptides can be produced by fragmenting a PROK2 or PROK1 polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

Regardless of the particular nucleotide sequence of a variant PROK2 or PROK1 gene, the gene encodes a polypeptide that may be characterized by its ability to bind specifically to an anti-PROK2 or anti-PROK1 antibody.

### 3. Production of PROK Antibodies

The antibody or antibody fragments of the present invention can be produced in recombinant host cells, including mammalian, bacterial, insect, and fungal cells, following conventional techniques.

Expression vectors that are suitable for production of a foreign protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence. As discussed above, expression vectors can also include nucleotide sequences encoding a secretory sequence that directs the heterologous polypeptide into the secretory pathway of a host cell. For example, a PROK2 expression vector may comprise a PROK2 gene and a secretory sequence derived from a PROK2 gene or another secreted gene.

PROK2 or PROK1 antibodies and antibody fragments of the present invention may be expressed in mammalian cells. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 [Chasin et al., Som. Cell. Molec. Genet. 12:555 1986]), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (1H-3T3; ATCC CRL 1658).

For a mammalian host, the transcriptional and translational regulatory signals may be derived from viral sources, such as adenovirus, bovine papilloma virus, simian virus, or the like, in which the regulatory signals are associated with a particular gene which has a high level of expression. Suitable transcriptional and translational regulatory sequences also can be obtained from mammalian genes, such as actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene (Hamer et al., J. Molec. Appl. Genet. 1:273 (1982)), the TK promoter of Herpes virus (McKnight, Cell 31:355 (1982)), the SV40 early promoter (Benoist et al., Nature 290:304 (1981)), the Rous sarcoma virus promoter (Gorman et al., Proc. Natl. Acad. Sci. USA 79:6777 (1982)), the cytomegalovirus promoter (Foecking et al., Gene 45:101 (1980)), and the mouse mammary tumor virus promoter (see, generally, Etcheverry, “Expression of Engineered Proteins in Mammalian Cell Culture,” in Protein Engineering: Principles and Practice, Cleland et al. (eds.), pages 163-181 (John Wiley & Sons, Inc. 1996)).

Alternatively, a prokaryotic promoter, such as the bacteriophage T3 RNA polymerase promoter, can be used to control PROK2 or PROK1 gene expression in mammalian cells if the prokaryotic promoter is regulated by a eukaryotic promoter (Zhou et al., Mol. Cell. Biol. 10:4529 (1990), and Kaufman et al., Nucl. Acids Res. 19:4485 (1991)).

An expression vector can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

PROK2 or PROK1 antibodies and antibody fragments can also be produced by cultured mammalian cells using a viral delivery system. Exemplary viruses for this purpose include adenovirus, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161 (1994), and Douglas and Curiel, Science & Medicine 4:44 (1997)). Advantages of the adenovirus system include the accommodation of relatively large DNA inserts, the ability to grow to high-titer, the ability to infect a broad range of mammalian cell types, and flexibility that allows use with a large number of available vectors containing different promoters.

Established techniques for producing recombinant proteins in baculovirus systems are provided by Bailey et al., “Manipulation of Baculovirus Vectors,” in Methods in Molecular Biology, Volume 7: Gene Transfer and Expression Protocols, Murray (ed.), pages 147-168 (The Humana Press, Inc. 1991), by Patel et al., “The baculovirus expression system,” in DNA Cloning 2: Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering: Principles and Practice, Cleland et al. (eds.), pages 183-218 (John Wiley & Sons, Inc. 1996).

Fungal cells, including yeast cells, can also be used to express the genes described herein. Yeast species of particular interest in this regard include Saccharomyces cerevisiae, Pichia pastoris, and Pichia methanolica. Suitable promoters for expression in yeast include promoters from GAL1 (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp17, YEp vectors such as YEp13 and YCp vectors, such as YCp19. Methods for transforming S. cerevisiae cells with exogenous DNA and producing recombinant polypeptides therefrom are disclosed by, for example, Kawasaki, U.S. Pat. No. 4,599,311, Kawasaki et al., U.S. Pat. No. 4,931,373, Brake, U.S. Pat. No. 4,870,008, Welch et al., U.S. Pat. No. 5,037,743, and Murray et al., U.S. Pat. No. 4,845,075. Transformed cells are selected by phenotype determined by the selectable marker, commonly drug resistance or the ability to grow in the absence of a particular nutrient (e.g., leucine). A suitable vector system for use in Saccharomyces cerevisiae is the POT1 vector system disclosed by Kawasaki et al. (U.S. Pat. No. 4,931,373), which allows transformed cells to be selected by growth in glucose-containing media. Additional suitable promoters and terminators for use in yeast include those from glycolytic enzyme genes (see, e.g., Kawasaki, U.S. Pat. No. 4,599,311, Kingsman et al., U.S. Pat. No. 4,615,974, and Bitter, U.S. Pat. No. 4,977,092) and alcohol dehydrogenase genes. See also U.S. Pat. Nos. 4,990,446, 5,063,154, 5,139,936, and 4,661,454.

Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459 (1986), and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

For example, the use of Pichia methanolica as host for the production of recombinant proteins is disclosed by Raymond, U.S. Pat. No. 5,716,808, Raymond, U.S. Pat. No. 5,736,383, Raymond et al., Yeast 14:11-23 (1998), and in international publication Nos. WO 97/17450, WO 97/17451, WO 98/02536, and WO 98/02565. DNA molecules for use in transforming P. methanolica will commonly be prepared as double-stranded, circular plasmids, which can be linearized prior to transformation. For polypeptide production in P. methanolica, the promoter and terminator in the plasmid can be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A suitable selectable marker for use in Pichia methanolica is a P. methanolica ADE2 gene, which encodes phosphoribosyl-5-aminoimidazole carboxylase (AIRC; EC 4.1.1.21), and which allows ade2 host cells to grow in the absence of adenine. For large-scale, industrial processes where it is desirable to minimize the use of methanol, it is possible to use host cells in which both methanol utilization genes (AUG1 and AUG2) are deleted. For production of secreted proteins, host cells can be used that are deficient in vacuolar protease genes (PEP4 and PRB1). Electroporation is used to facilitate the introduction of a plasmid containing DNA encoding a polypeptide of interest into P. methanolica cells. P. methanolica cells can be transformed by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Expression vectors can also be introduced into plant protoplasts, intact plant tissues, or isolated plant cells. Methods for introducing expression vectors into plant tissue include the direct infection or co-cultivation of plant tissue with Agrobacterium tumefaciens, microprojectile-mediated delivery, DNA injection, electroporation, and the like. See, for example, Horsch et al., Science 227:1229 (1985), Klein et al., Biotechnology 10:268 (1992), and Miki et al., “Procedures for Introducing Foreign DNA into Plants,” in Methods in Plant Molecular Biology and Biotechnology, Glick et al. (eds.), pages 67-88 (CRC Press, 1993).

Alternatively, genes encoding the antibodies or antibody fragments can be expressed in prokaryotic host cells. Suitable promoters that can be used to express PROK2 or PROK1 polypeptides in a prokaryotic host are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, lpp-lacSpr, phoa, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters have been reviewed by Glick, J. Ind. Microbiol. 1:277 (1987), Watson et al., Molecular Biology of the Gene, 4th Ed. (Benjamin Cummins 1987), and by Ausubel et al. (1995).

Suitable prokaryotic hosts include E. coli and Bacillus subtilus. Suitable strains of E. coli include BL21(DE3), BL21(DE3)pLysS, BL21(DE3)pLysE, DH1, DH4I, DH5, DH5I, DH5IF′, DH5IMCR, DH10B, DH10B/p3, DH11S, C600, HB101, JM101, JM105, JM109, JM110, K38, RR1, Y1088, Y1089, CSH18, ER1451, and ER1647 (see, for example, Brown (ed.), Molecular Biology Labfax (Academic Press 1991)). Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)).

When expressing an anti-PROK antibody or antibody fragment in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Methods for expressing proteins in prokaryotic hosts are well-known to those of skill in the art (see, for example, Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995), Ward et al., “Genetic Manipulation and Expression of Antibodies,” in Monoclonal Antibodies: Principles and Applications, page 137 (Wiley-Liss, Inc. 1995), and Georgiou, “Expression of Proteins in Bacteria,” in Protein Engineering: Principles and Practice, Cleland et al. (eds.), Chapter 4, starting at page 101 (John Wiley & Sons, Inc. 1996), and Rudolph, “Successful Refolding on an Industrial Scale”, Chapter 10).

Standard methods for introducing expression vectors into bacterial, yeast, insect, and plant cells are provided, for example, by Ausubel (1995).

General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2: Expression Systems, 2nd Edition, Glover et al. (eds.), pages 59-92 (Oxford University Press 1995). Established methods for isolating recombinant proteins from a baculovirus system are described by Richardson (ed.), Baculovirus Expression Protocols (The Humana Press, Inc. 1995).

As an alternative, antibodies or antibody fragments of the present invention can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat'l Acad. Sci. USA 94:7845 (1997), Dawson, Methods Enzymol. 287: 34 (1997), Muir et al, Proc. Nat'l Acad. Sci. USA 95:6705 (1998), and Severinov and Muir, J. Biol. Chem. 273:16205 (1998)).

Antibodies and antibody fragments bind peptides and polypeptides of the present invention comprise at least six, at least nine, or at least 15 contiguous amino acid residues of SEQ ID NOs:2 and 5. Illustrative polypeptides of PROK1, for example, include 15 contiguous amino acid residues of amino acids 82 to 105 of SEQ ID NO:5. Exemplary polypeptides of PROK2 include 15 contiguous amino acid residues of amino acids 1 to 32 or amino acids 75 to 108 of SEQ ID NO:2, whereas exemplary PROK1 polypeptides include amino acids 82 to 105 of SEQ ID NO:5. Within certain embodiments of the invention, the polypeptides comprise 20, 30, 40, 50, 75, or more contiguous residues of SEQ ID NOs:2 or 5. Nucleic acid molecules encoding such peptides and polypeptides are useful as polymerase chain reaction primers and probes.

Antibodies to a PROK polypeptide can be obtained, for example, using the product of a PROK expression vector or PROK isolated from a natural source as an antigen. Particularly useful anti-PROK2 and anti-PROK1 antibodies “bind specifically” with PROK2 and PROK1, respectively. Antibodies are considered to be specifically binding if the antibodies exhibit at least one of the following two properties: (1) antibodies bind to PROK2 and/or PROK1 with a threshold level of binding activity, and (2) antibodies do not significantly cross-react with polypeptides related to PROK2 or PROK1.

With regard to the first characteristic, antibodies specifically bind if they bind to a PROK polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660 (1949)). With regard to the second characteristic, antibodies do not significantly cross-react with related polypeptide molecules, for example, if they detect PROK, but not known polypeptides using a standard Western blot analysis. Particular anti-PROK2 antibodies bind PROK2, but not PROK1, while certain anti-PROK1 antibodies bind PROK1, but not PROK2.

In addition, an antibody or variant or fragment thereof, that binds to both PROK2 and PROK1 may be useful as an antagonist of the anti-angiogenesis, anti-tumor, anti-vascularization, anti-contractility, and anti-inflammation described herein.

Anti-PROK2 and anti-PROK1 antibodies can be produced using antigenic PROK2 or PROK1 epitope-bearing peptides and polypeptides. Antigenic epitope-bearing peptides and polypeptides of the present invention contain a sequence of at least four, or between 15 to about 30 amino acids contained within SEQ ID NOs:2, 29, or 5. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies that bind with PROK2 or PROK1. It is desirable that the amino acid sequence of the epitope-bearing peptide is selected to provide substantial solubility in aqueous solvents (i.e., the sequence includes relatively hydrophilic residues, while hydrophobic residues are preferably avoided). Moreover, amino acid sequences containing proline residues may be also be desirable for antibody production.

As an illustration, potential antigenic sites in PROK2 or PROK1 were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

The Jameson-Wolf method predicts potential antigenic determinants by combining six major subroutines for protein structural prediction. Briefly, the Hopp-Woods method, Hopp et al., Proc. Nat'l Acad. Sci. USA 78:3824 (1981), was first used to identify amino acid sequences representing areas of greatest local hydrophilicity (parameter: seven residues averaged). In the second step, Emini's method, Emini et al., J. Virology 55:836 (1985), was used to calculate surface probabilities (parameter: surface decision threshold (0.6)=1). Third, the Karplus-Schultz method, Karplus and Schultz, Naturwissenschaften 72:212 (1985), was used to predict backbone chain flexibility (parameter: flexibility threshold (0.2)=1). In the fourth and fifth steps of the analysis, secondary structure predictions were applied to the data using the methods of Chou-Fasman, Chou, “Prediction of Protein Structural Classes from Amino Acid Composition,” in Prediction of Protein Structure and the Principles of Protein Conformation, Fasman (ed.), pages 549-586 (Plenum Press 1990), and Garnier-Robson, Garnier et al., J. Mol. Biol. 120:97 (1978) (Chou-Fasman parameters: conformation table=64 proteins; a region threshold=103; β region threshold=105; Garnier-Robson parameters: α and β decision constants=0). In the sixth subroutine, flexibility parameters and hydropathy/solvent accessibility factors were combined to determine a surface contour value, designated as the “antigenic index.” Finally, a peak broadening function was applied to the antigenic index, which broadens major surface peaks by adding 20, 40, 60, or 80% of the respective peak value to account for additional free energy derived from the mobility of surface regions relative to interior regions. This calculation was not applied, however, to any major peak that resides in a helical region, since helical regions tend to be less flexible.

The results of this analysis indicated that suitable antigenic peptides of PROK2 include the following segments of the amino acid sequence of SEQ ID NO:2: amino acids 22 to 27 (“antigenic peptide 1”), amino acids 33 to 41 (“antigenic peptide 2”), amino acids 61 to 68 (“antigenic peptide 3”), amino acids 80 to 85 (“antigenic peptide 4”), amino acids 97 to 102 (“antigenic peptide 5”), and amino acids 61 to 85 (“antigenic peptide 6”). The present invention contemplates the use of any one of antigenic peptides 1 to 6 to generate antibodies to PROK2. The present invention also contemplates polypeptides comprising at least one of antigenic peptides 1 to 6.

Similarly, analysis of the PROK1 amino acid sequence indicated that suitable antigenic peptides of PROK1 include the following segments of the amino acid sequence of SEQ ID NO:5: amino acids 25 to 33 (“antigenic peptide 7”), amino acids 53 to 66 (“antigenic peptide 8”), amino acids 88 to 95 (“antigenic peptide 9”), amino acids 98 to 103 (“antigenic peptide 10”), and amino acids 88 to 103 (“antigenic peptide 11”). The present invention contemplates the use of any one of antigenic peptides 7 to 11 to generate antibodies to PROK1. The present invention also contemplates polypeptides comprising at least one of antigenic peptides 7 to 11.

Polyclonal antibodies to recombinant PROK protein or to PROK isolated from natural sources can be prepared using methods well-known to those of skill in the art. See, for example, Green et al., “Production of Polyclonal Antisera,” in Immunochemical Protocols (Manson, ed.), pages 1-5 (Humana Press 1992), and Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995). The immunogenicity of a PROK polypeptide can be increased through the use of an adjuvant, such as alum (aluminum hydroxide) or Freund's complete or incomplete adjuvant. Polypeptides useful for immunization also include fusion polypeptides, such as fusions of PROK or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an anti-PROK antibody of the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310 (1990).

Alternatively, monoclonal anti-PROK antibodies can be generated. Rodent mono-clonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.1-2.6.7 (John Wiley & Sons 1991) [“Coligan”], Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising a PROK gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

Hybridomas expressing the neutralizing monoclonal antibodies to human PROK2 described above were deposited with the American Type Tissue Culture Collection (ATCC; Manassas Va.) patent depository as original deposits under the Budapest Treaty and were given the following ATCC Accession No.s: clone 279.111.5.2 (ATCC Patent Deposit Designation PTA-6856, deposited on Jul. 13, 2005); clone 279.121.7.4 (ATCC Patent Deposit Designation PTA-6859, deposited on Jul. 13, 2005); clone 279.124.1.4 (ATCC Patent Deposit Designation PTA-6857, deposited on Jul. 13, 2005); and clone 279.126.5.6.5 (ATCC Patent Deposit Designation PTA-6858; deposited on Jul. 13, 2005).

In addition, an anti-PROK antibody of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)).

For particular uses, it may be desirable to prepare fragments of anti-PROK antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230 (1960), Porter, Biochem. J. 73:119 (1959), Edelman et al., in Methods in Enzymology Vol. 1, page 422 (Academic Press 1967), and by Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Nat'l Acad. Sci. USA 69:2659 (1972). Alternatively, the variable chains can be linked by an intermolecular disulfide bond or cross-linked by chemicals such as glutaraldehyde (see, for example, Sandhu, Crit. Rev. Biotech. 12:437 (1992)).

The Fv fragments may comprise V_{H} and V_{L} chains, which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97 (1991) (also see, Bird et al., Science 242:423 (1988), Ladner et al., U.S. Pat. No. 4,946,778, Pack et al., Bio/Technology 11:1271 (1993), and Sandhu, supra).

As an illustration, a scFV can be obtained by exposing lymphocytes to PROK polypeptide in vitro, and selecting antibody display libraries in phage or similar vectors (for instance, through use of immobilized or labeled PROK protein or peptide). Genes encoding polypeptides having potential PROK polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides, which interact with a known target that can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the PROK sequences disclosed herein to identify proteins which bind to PROK.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106 (1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an anti-PROK antibody may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986), Carter et al., Proc. Nat'l Acad. Sci. USA 89:4285 (1992), Sandhu, Crit. Rev. Biotech. 12:437 (1992), Singer et al., J. Immun. 150:2844 (1993), Sudhir (ed.), Antibody Engineering Protocols (Humana Press, Inc. 1995), Kelley, “Engineering Therapeutic Antibodies,” in Protein Engineering Principles and Practice, Cleland et al. (eds.), pages 399-434 (John Wiley & Sons, Inc. 1996), and by Queen et al., U.S. Pat. No. 5,693,762 (1997).

Polyclonal anti-idiotype antibodies can be prepared by immunizing animals with anti-PROK antibodies or antibody fragments, using standard techniques. See, for example, Green et al., “Production of Polyclonal Antisera,” in Methods In Molecular Biology: Immunochemical Protocols, Manson (ed.), pages 1-12 (Humana Press 1992). Also, see Coligan at pages 2.4.1-2.4.7. Alternatively, monoclonal anti-idiotype antibodies can be prepared using anti-PROK antibodies or antibody fragments as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J. Gen. Virol. 77:1875 (1996).

### 4. Therapeutic Uses of PROK Polypeptides and Antibodies

The present invention includes the use of anti-PROK molecules, including antagonists, antibodies, binding proteins, variants and fragments, having anti-PROK activity. The invention includes administering to a subject, the anti-PROK molecule and contemplates both veterinary and human therapeutic uses. Illustrative subjects include mammalian subjects, such as farm animals, domestic animals, and human patients.

Anti-PROK molecules, antagonists, antibodies, binding proteins, variants and fragments, are useful in treating and detecting Inflammatory Bowel Disease (IBD) and Irritable Bowel Syndrome (IBS), cancer, tumor size and proression, angiogenesis and vascularization disorders.

Inflammatory Bowel Disease (IBD) can affect the colon and/or rectum (Ulcerative colitis), or the small and large intestine (Crohn's Disease). The pathogenesis of these diseases is unclear, but they involve chronic inflammation of the affected tissues. Potential therapeutics include anti-PROK molecules, including, anti-PROK2 and anti-PROK1 antibodies, other binding proteins, variants, fragments, chimeras, and other PROK2 and PROK1 antagonists. These molecules could serve as a valuable therapeutic to reduce inflammation and pathological effects in IBD and related diseases.

Ulcerative colitis (UC) is an inflammatory disease of the large intestine, commonly called the colon, characterized by inflammation and ulceration of the mucosa or innermost lining of the colon. This inflammation causes the colon to empty frequently, resulting in diarrhea. Symptoms include loosening of the stool and associated abdominal cramping, fever and weight loss. Although the exact cause of UC is unknown, recent research suggests that the body's natural defenses are operating against proteins in the body which the body thinks are foreign (an “autoimmune reaction”). Perhaps because they resemble bacterial proteins in the gut, these proteins may either instigate or stimulate the inflammatory process that begins to destroy the lining of the colon. As the lining of the colon is destroyed, ulcers form, releasing mucus, pus and blood. The disease usually begins in the rectal area and may eventually extend through the entire large bowel. Repeated episodes of inflammation lead to thickening of the wall of the intestine and rectum with scar tissue. Death of colon tissue or sepsis may occur with severe disease. The symptoms of ulcerative colitis vary in severity and their onset may be gradual or sudden. Attacks may be provoked by many factors, including respiratory infections or stress. Thus, the anti-PROK molecules of the present invention can be useful to treat and or detect UC.

Although there is currently no cure for UC available, treatments are focused on suppressing the abnormal inflammatory process in the colon lining. Treatments including corticosteroids immunosuppressives (e.g. azathioprine, mercaptopurine, and methotrexate) and aminosalicytates are available to treat the disease. However, the long-term use of immunosuppressives such as corticosteroids and azathioprine can result in serious side effects including thinning of bones, cataracts, infection, and liver and bone marrow effects. In the patients in whom current therapies are not successful, surgery is an option. The surgery involves the removal of the entire colon and the rectum.

There are several animal models that can partially mimic chronic ulcerative colitis. The most widely used model is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanied by the clinical picture of progressive weight loss (wasting), bloody diarrhea, rectal prolapse, and large bowel wall thickening (Neurath et al. Intem. Rev. Immunol. 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. DSS is regarded as a T cell-independent model because it is observed in T cell-deficient animals such as SCID mice.

The administration of anti-PROK2 or znti-PROK1 antibodies or binding partners to these TNBS or DSS models can be used to ameliorate symptoms and alter the course of gastrointestinal disease. PROK2 and/or PROK1 may play a role in the inflammatory response in colitis, and the neutralization of PROK2 and/or PROK1 activity by administrating antagonists is a potential therapeutic approach for IBD.

Inflammatory reactions cause various clinical manifestations frequently associated with abnormal motility of the gastrointestinal tract, such as nausea, vomiting, ileus or diarrhea. Bacterial lipopolysaccharide (LPS) exposure, for example, induces such an inflammatory condition, which is observed in both humans and experimental animals, and is characterized by biphasic changes in gastrointestinal motility: increased transit in earlier phases and delayed transit in later phases. Since PROK2 plays a role in inflammation, and has biphasic activities at low (prokinetic) and high (inhibitory) doses, it will be beneficial in these inflammatory conditions.

Irritable Bowel Syndrome is one of the most common conditions in the gastrointestinal clinic. Yet, diagnosis and treatment for IBS remain limited. As the expression of PROK2 has been correlated with symptoms of IBS, anti-PROK molecules, including, anti-PROK2 and anti-PROK1 antibodies, other binding proteins, variants, fragments, chimeras, and other PROK2 and PROK1 antagonists are useful in reducing symptoms and treatment of the disease.

Additional characteristic of IBS are impaired gastrointestinal motility, with symptoms often alternating between bouts of diarhea and constipation, and increased visceral sensitivity to intestinal smooth muscle contractions and distention. As PROK2 and PROK1 are molecules that regulate gastrointestinal contractiliy, gastric emptying and intestinal transit, PROK polypeptides, such as PROK2, PROK1, as well as agonists, fragments, variants and/or chimeras, of the present invention can be particularly useful in an overall treatment for IBS. The biphasic nature of PROK2, i.e., its ability to inhibit motility at high doses, and enhance motility at low doses, suggest that its expression is dys-regulated in IBS, with constipation prone patients displaying elevated PROK2 levels, and diarrhea prone patients displaying lower PROK2 levels.

The administration of anti-PROK2 or znti-PROK1 antibodies or binding partners to a patient with IBD or IBS can be used to ameliorate symptoms and alter the course of gastrointestinal disease. PROK2 and/or PROK1 may play a role in the inflammatory response in colitis, and the neutralization of PROK2 and/or PROK1 activity by administrating antagonists is a potential therapeutic approach for IBD and/or IBS.

PROK polypeptides, such as PROK2, PROK1, as well as agonists, fragments, variants and/or chimeras thereof, can be used to stimulate chemokine production. Chemokines are small pro-inflammatory proteins that have a broad range of activities involved in the recruitment and function of leukocytes. Rat CINC-1, murine KC, and human GROα are members of the CXC subfamily of chemokines. Chemokines, in general, can be divided into groups that are chemotactic predominately for neutrophils, and also have angiogenic activity, and those that primarily attract T lymphocytes and monocytes. See Banks, C. et al, J. Pathology 199: 28-35, 2002. Chemokines in the first group display an ELR (Glu-Leu-Arg) amino acid motif at the NH₂ terminus. GROα, for example, contains this motif. GROα also has mitogenic and angiogenic properties and is involved in wound healing and blood vessel formation. (See, for example, Li and Thornhill, Cytokine 12:1409 (2000)). As illustrated by Examples 2, 3, and 11, PROK2 and PROK1 stimulated the release of chemokine CINC-1 (Cytokine Induced Neutrophil Chemoattractant factor 1) in cell lines derived from the thoracic aorta of rats, PROK2 stimulated the release of chemokine KC from mice, and chemokine MIP-2 (mouse Macrophage Inflammatory Protein-2) is up-regulated in response to a low dose (intraperitoneal injection) of PROK2. Therefore, PROK polypeptides, such as PROK2, PROK1, as well as agonists, fragments, variants and/or chimeras thereof, can be used to stimulate the production chemokines in vivo. The chemokines can be purified from culture media and used in research or clinical settings. PROK variants can also be identified by the ability to stimulate production of chemokines in vitro or in vivo.

Upregulated chemokine expression correlates with increasing activity of IBD. See Banks, C. et al, J. Pathology 199: 28-35, 2002. Chemokines are able to attract inflammatory cells and are involved in their activation. Similarly, MIP-2 expression has been found to be associated with neutrophil influx in various inflammatory conditions. As polypeptides that stimulate the production of chemokines, PROK polypeptides, such as PROK2, PROK1, as well as agonists, fragments, variants and/or chimeras thereof, may be useful in treating Inflammatory Bowel Disease by reducing, inhibiting or preventing chemokine influx in the intestinal tract.

As a protein that can stimulate the production of chemokines, PROK polypeptides, such as PROK2, PROK1, as well as agonists, fragments, variants and/or chimeras thereof, may be useful in treating infections, including fungal, bacterial, viral and parasitic infections. Thus, the administration of a PROK polypeptide, such as PROK2, PROK1, as well as an agonist, fragment, variant and/or a chimera thereof, may be used as an immune booster to a specific tissue site. For example, PROK2 administered to gastrointestinal tissue, or to lung tissue, may be useful alone, or in combination therapy to treat infections.

As shown in Example 3, PROK2 administration can cause neutrophil infiltration. There are many aspects involved in the immune response of a mammal to an injury or infection where neutrophil infiltration would be desirable. As such, PROK polypeptides, such as PROK2, PROK1, as well as agonists, fragments, variants and/or chimeras thereof, will be useful as an agent to induce neutrophil infiltration.

The additional activity of PROK2 as a modulator of immunity and chemotaxis, inducing neutrophil infiltration, indicates that it may be involved in the early infectious insults that are often the initiator of IBS (Collins et al). By both increasing intestinal motility and inducing neutrophil influx to remove invading pathogens, PROK2 would serve to resolve a gastrointestinal infection such as food poisoning. In some IBS patients, this infectious event is never resolved, leading to a chronic inflammatory state and gastrointestinal motility problems, either constipation or diarrhea, or alternating bouts of both. A PROK2 inhibitor could additionally reduce the inflammatory state, by reducing neutrophil numbers in affected inflamed gastrointestinal tissue.

Inflammatory reactions cause various clinical manifestations frequently associated with abnormal motility of the gastrointestinal tract, such as nausea, vomiting, ileus or diarrhea. Bacterial lipopolysaccharide (LPS) exposure, for example, induces such an inflammatory condition, which is observed in both humans and experimental animals, and is characterized by biphasic changes in gastrointestinal motility: increased transit in earlier phases and delayed transit in later phases. Since PROK2 plays a role in inflammation, and has biphasic activities at low (prokinetic) and high (inhibitory) doses, it will be beneficial in these inflammatory conditions.

For disorders related to IBS and IBD, clinical signs of improved function include, but are not limited to, reduction in pain, cramping and sensitivity, reduction in diarrhea and improved stool consistency, reduced abdominal distension, and increased intestinal transit. Improvement can also be measured by a decrease in mean Crohn's Disease Activity Index (CDAI). See Best. W. et al., Gasttoenterology 70: 439-44, 1976. Additionally, improved function can be measured by a quality of life assessment as described by Irvine et al. (Irvine, E. et al., Gasttoenterology 106: 287-96, 1994.

For disorders related to deficient gastrointestinal function, clinical signs of improved function include, but are not limited to, increased intestinal transit, increased gastric emptying, flatus, and borborygmi, ability to consume liquids and solids, and/or a reduction in nausea and/or emesis

For disorders related to hyperactive gastrointestinal contractility, clinical signs of improved gastrointestinal function include, but are not limited to, slowed gastric emptying, slowed intestinal transit, and/or a reduction in cramps associated with diarrhea.

PROK polypeptides, such as PROK2, PROK1, as well as agonists, fragments, variants and/or chimeras thereof, can also be used to treat gastrointestinal related sepsis. Experimental “sepsis”/endotoxemia is produced in rodents using methods described in Ceregrzyn et al. Neurogastroenterol. Mot. 13:605-613 (2001). These animals develop biphasic alterations in gastrointestinal transit. A PROK polypeptide, such as PROK2, PROK1, as well as agonists, fragments, variants and/or chimeras thereof, can be administered orally (p.o.), intraperitoneally (i.p.), intraveneously (i.v.), subcutaneously (s.c.), or intramuscularly (i.m.) at either low (prokinetic) or high (inhibitory) concentrations, depending on the phase of the disease. Gastric emptying and/or intestinal transit would then be measured using one of the Major Models described below.

As shown in the Examples, PROK2 induces the release of GROα. There are several inflammatory disorders diseases associated with GROα production, such as inflammation, neoplasms, and other disease. For example, the inflammatory disease include but are not limited to, psoriasis, ulcerative colitis, rheumatoid arthritis, bacterial pneumonica, and adult respiratory distress syndrome. Models associated with GROα increases in inflammation include an endotoxin-induced uvetis model, an air pouch-type allergic inflammation model, a monosodium urate pleurisy model, an antiglomerular basement membrane (GBM) glomerulonephritis model, a LPS-induced endotoxemia model, a Type II collagen-induced arthritis model, a bacterial meningitis model, an experimental allergic encephalomyelitis model and an acute lung inflammation model. See for example, Aggarwal, B., “Human Cytokines: Handbook for Basic and Clinical Research, Vol. III, page 294-295.

The neoplastic diseases associated with GROα production, such as but not limited to squamous cell carcinoma, melanoma, basal cell carcinoma, and colon carcinoma. Models associated with GROα increases in neoplasm include melanoma, HTLV-1 T-cell leukemia, and angiogenesis. See for example, Aggarwal, B., “Human Cytokines: Handbook for Basic and Clinical Research, Vol. III, page 294-295.

The injury diseases associated with GROα production include verruca vulgaris, keratonacanthoma and viral infection (such as HIV). Models associated with injury include ischemia (cerebral and renal), hepatotoxicity (ethanol, cadmium), and wound healing. See for example, Aggarwal, B., “Human Cytokines: Handbook for Basic and Clinical Research, Vol. III, page 294-295.

PROK2 is also expressed in leukocytes (neutrophils), testis, and brain and is upregulated post hypoxic stress, which induces angiogenic factors. As such, an antagonist is useful to treat or reduce the symptoms of diseases that are associated with hypoxic stress. Such diseases are readily known.

Since chemokines can promote and accelerate tissue repair, such as PROK2, PROK1, as well as agonists, fragments, variants and/or chimeras thereof, can have a beneficial role in resolving disease. For example, topical administration is useful for wound healing applications, including the prevention of excess scaring and granulation tissue, prevention of keyloids, and prevention of adhesions following surgery.

A number of in vivo models can be used to evaluate the anti-inflammation, anti-gastric emptying, and anti-intestinal transit effects of the PROK antagonists described herein. For example, Wirtz and Neurath describe spontaneous and inducible models of Inflammatory Bowel Disease (IBD). See Wirtz and Neurath. Int J. Colorectal Dis. 15:144-60 (2000). Similarly, Mayer and Collins describe in vivo models of irritable bowel syndrome (IBS), including pain assessment, intestinal transit and gastric emptying. See Mayer and Collins. Gastroenterol. 122:2032-2048 (2002). See also Puig and Pol. J. Pharmacol. Experiment. Therap. 287:1068 (1998); and Takeuchi et al. Digest. Dis. Sci. 42; 251-258 (1997); Trudel et al Peptides 24:531-534 (2003); Martinez et al. J. Pharmacol. Experiment. Ther. 301: 611-617 (2002); Takeda et al. Jpn. J. Pharmacol. 81:292-297 (1999); and Yoshida. and Ito. J. Pharmacol. Experiment. Therap. 257, 781-787 (1991) and Furuta et al. Biol. Pharm. Bull. 25:103-1071 (2002). In addition, models to assess emesis are well known in the art.

### 5. General Models of Inflammation

High chemokine levels and neutrophil infiltrates are characteristics of local acute inflammation. Epithelial cell damage and infiltration by neutrophils is especially prominent in the local inflammatory process of ulcerative colitis. PROK2 antagonists or PROK1 antagonists, therefore, can be used as anti-inflammatory agents, including inflammation associated with cells or tissues. As an illustration, a PROK2 antagonist can be used as an anti-inflammatory agent to treat inflammatory bowel diseases associated with increased neutrophil infiltration, or chemokine expression (e.g., Crohn's disease, ulcerative colitis, and irritable bowel syndrome). A PROK2 antagonist can also be used to treat inflammation of the brain (e.g., associated with encephalomyelitis, multiple sclerosis, and the like). An illustrative PROK2 antagonist is an antibody or antibody fragment that binds with a polypeptide having the amino acid sequence of amino acid residues 23 to 108 of SEQ ID NO:2, with a polypeptide having the amino acid sequence of amino acid residues 28 to 108 of SEQ ID NO:2, or with a polypeptide having the amino acid sequence of amino acid residues 20 to 105 of SEQ ID NO:5. The monoclonal antibodies described herein can be used as anti-inflammatory agents to treat inflammatory diseases associated with neutrophil and/or chemokine expression.

Neuropathy and sensory deficiency involve pain and loss of sensitivity, and can be related to such diseases as, diabetes, multiple sclerosis, and hypertension, for example. As a protein that is expressed in the brain, antagonists of PROK2 may be useful to treat pain and sensory deficiencies. For example, PROK2 antagonists can be delivered topically, centrally, or systemically, to treat diabetic neuropathy. The monoclonal antibodies described herein can be used as to treat pain associated with neuropathy and pain.

PROK2 polypeptides, and other PROK2 agonists, can be used to enhance the immune function in, for example, patients with various forms of cancer, angiogenesis, tumor growth, and inflammation associated with cancer cells or tissues, HIV infection, or an immune disorder, such as chronic granulomatous disease or Chedick Higashi Syndrome. PROK2 polypeptides, and other PROK2 agonists, can also be used to alleviate pain, such as visceral pain or severe headache (e.g., migraine).

### 6. General Models of Angiogenesis

As shown in Example 5, PROK2 and PROK1 can stimulate angiogenesis. Accordingly, PROK2, PROK1, PROK2 agonists, and PROK1 agonists can be used to induce growth of new blood vessels. These molecules can be administered to a mammalian subject alone or in combination with other angiogenic factors, such as vascular endothelial growth factor.

In vitro models to measure the anti-antiogenic effects of the antibodies and antagonists of the present invention include the rat aortic ring outgrowth assay, the tube formation assay, the microcarrier sprouting assay, all of which are well-known in the art.

In vivo models to measure the anti-angiogenic effects of the antibodies and antagonists of the present invention include the dorsal airsac model (using transiently and stably transfected cell lines to express the PROK ligands in nude mice), the matrigel assay, the rat cornel model, and injection adenovirus containing the PROK gene in selected tissues such as testes and ovary.

PROK2 and PROK1 polypeptides for the methods of the present invention are shown to stimulate angiogenesis in animal models. Thus, the monoclonal antibodies of the present invention will be useful in decreased tumor burden and tumor cells, and increased survival, and can hence be used in therapeutic anti-cancer applications in humans. As such, anti-PROK2 and anti-PROK1 anti-cancer activity is useful in the treatment and prevention of human cancers. Such indications include but are not limited to the following: Carcinomas (epithelial tissues), Sarcomas of the soft tissues and bone (mesodermal tissues), Adenomas (glandular tissues), cancers of all organ systems, such as liver (hepatoma) and kidney (renal cell carcinomas), CNS (gliomas, neuroblastoma), and hematological cancers, viral associated cancers (e.g., associated with retroviral infections, HPV, hepatitis B and C, and the like), lung cancers, endocrine cancers, gastrointestinal cancers (e.g., biliary tract cancer, liver cancer, pancreatic cancer, stomach cancer and colorectal cancer), genitourinary cancers (e.g., prostate cancer bladder cancer, renal cell carcinoma), gynecologic cancers (e.g., uterine cancer, cervical cancer, ovarian cancer) breast, and other cancers of the reproductive system, head and neck cancers, and others. Of particular interest are hematopoietic cancers, including but not limited to, lymphocytic leukemia, myeloid leukemia, Hodgkin's lymphoma, Non-Hodgkins lymphomas, chronic lymphocytic leukemia, AML, and other leukemias and lymphomas. Moreover PROK2 can be used therapeutically in cancers of various non-metastatic as wells as metastatic stages such as “Stage 1” Localized (confined to the organ of origin); “Stage 2” Regional; “Stage 3” Extensive; and “Stage 4” Widely disseminated cancers. In addition, anti-PROK2 and anti-PROK1 antibodies can be used in various applications for cancer, immunotherapy, and in conjunction with chemotherapy and the like.

### 7. General Tumor Models

Models of tumor progression consist of models of tumor cell lines and in vivo models. The tumor cell line models are readily known in the art and include, for example, the EG7 mouse thymoma cell line, the P815 mouse mastocytom cell line, the HT29 human colorectal adenocarcinoma cell line, the SW620 human colorectal adenocarcinoma cell line, the CT26 mouse colon carcinoma cell line, the Renca mouse kidney carcinoma cell line, the B16 mouse melanoma cell line, the 4T1 cell line (when injected into BALB/c mice, 4T1 cell spontaneously produce highly metastatic tumors that can metastaisize to the lung, liver, lymph nodes and brain while the primary tumor is growing in situ. Class 4 breast cancer model), and the EMT6 cell line (which was established from a transplantable murine mammary carcinoma that arose in BALB/cCRGL mouse).

Models of tumor progression in solid tumors include but are not limited to, sub cutaneous tumor models (syngeneic and xenograft models), orthotopic tumor models (e.g. implantation in the cecum), and CD8+ stable expression of tumor cell lines.

There are several syngeneic mouse models that have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Appropriate tumor models for our studies include the Lewis lung carcinoma (ATCC No. CRL-1642) and B16 melanoma (ATCC No. CRL-6323), amongst others. These are both commonly used tumor lines, syngeneic to the C57BL6/J mouse, that are readily cultured and manipulated in vitro. Tumors resulting from implantation of either of these cell lines are capable of metastasis to the lung in C57BL6/J mice. The Lewis lung carcinoma model has recently been used in mice to identify an inhibitor of angiogenesis (O'Reilly M S, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves can be infected with recombinant adenovirus, such as one expressing PROK2 or PROK1, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500-1800 mm³ in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed PROK2 or PROK1, on the ability of the tumor to recruit vasculature and undergo metastasis can thus be assessed. In addition, aside from using adenovirus, the implanted cells can be transiently transfected with PROK2 or PROK1. Use of stable PROK2 or PROK1 transfectants as well as use of inducible promoters to activate PROK2 or PROK1 expression in vivo are known in the art and can be used in this system to assess PROK2 or PROK1 induction of metastasis. Moreover, purified PROK2 or PROK1 or PROK2 or PROK1 conditioned media can be directly injected in to this mouse model, and hence be used in this system. For general reference see, O'Reilly M S, et al. Cell 79:315-328, 1994; and Rusciano D, et al. Murine Models of Liver Metastasis. Invasion Metastasis 14:349-361, 1995.

The activity of PROK2 or PROK1 and its derivatives (conjugates) on growth and dissemination of tumor cells derived from human hematologic malignancies can be measured in vivo. Several mouse models have been developed in which human tumor cells are implanted into immunodeficient mice (collectively referred to as xenograft models); see, for example, Cattan A R, Douglas E, Leuk. Res. 18:513-22, 1994 and Flavell, D J, Hematological Oncology 14:67-82, 1996. The characteristics of the disease model vary with the type and quantity of cells delivered to the mouse, and several disease models are known in the art. In an example of this model, tumor cells (e.g. Raji cells (ATCC No. CCL-86)) would be passaged in culture and about 1×10⁶ cells injected intravenously into severe combined immune deficient (SCID) mice. Such tumor cells proliferate rapidly within the animal and can be found circulating in the blood and populating numerous organ systems. Therapies designed to kill or reduce the growth of tumor cells using PROK2 or PROK1 or its derivatives, agonists, conjugates or variants can be tested by administration of PROK2 or PROK1 compounds to mice bearing the tumor cells. Efficacy of treatment is measured and statistically evaluated as increased survival within the treated population over time. Tumor burden may also be monitored over time using well-known methods such as flow cytometry (or PCR) to quantitate the number of tumor cells present in a sample of peripheral blood. For example, therapeutic strategies appropriate for testing in such a model include direct treatment with PROK2 or PROK1 or related conjugates or antibody-induced toxicity based on the interaction of PROK2 or PROK1 with its receptor(s), or for cell-based therapies utilizing PROK2 or PROK1 or its derivatives, agonists, conjugates or variants. The latter method, commonly referred to as adoptive immunotherapy, would involve treatment of the animal with components of the human immune system (i.e. lymphocytes, NK cells, bone marrow) and may include ex vivo incubation of cells with PROK2 or PROK1 with or without other immunomodulatory agents described herein or known in the art.

The activity of PROK2 or PROK1 on immune (effector) cell-mediated tumor cell destruction can be measured in vivo, using the murine form or the human form of PROK2 (SEQ ID NO:2) or PROK1 protein in syngeneic mouse tumor models. Several such models have been developed in order to study the influence of polypeptides, compounds or other treatments on the growth of tumor cells and interaction with their natural host, and can serve as models for therapeutics in human disease. In these models, tumor cells passaged in culture or in mice are implanted into mice of the same strain as the tumor donor. The cells will develop into tumors having similar characteristics in the recipient mice. For reference, see, for example, van Elsas et al., J. Exp. Med. 190:355-66, 1999; Shrikant et al., Immunity 11:483-93, 1999; and Shrikant et al., J. Immunol. 162:2858-66, 1999. Appropriate tumor models for studying the activity of PROK2 or PROK1 on immune (effector) cell-mediated tumor cell destruction include the B16-F10 melanoma (ATCC No. CRL-6457), and the EG.7 thymoma (ATCC No. CRL-2113), described herein, amongst others. These are both commonly used tumor cell lines, syngeneic to the C57BL6 mouse, which are readily cultured and manipulated in vitro.

In an example of an in vivo model, the tumor cells (e.g. B16-F10 melanoma (ATCC No. CRL-6475) are passaged in culture and about 100,000 cells injected intravenously into C57BL6 mice. In this mode of administration, B16-F10 cells will selectively colonize the lungs. Small tumor foci are established and will grow within the lungs of the host mouse. Therapies designed to kill or reduce the growth of tumor cells using PROK2 or PROK1 or its derivatives, agonists, conjugates or variants can be tested by administration of compounds to mice bearing the tumor cells. Efficacy of treatment is measured and statistically evaluated by quantitation of tumor burden in the treated population at a discrete time point, two to three weeks following injection of tumor cells. Therapeutic strategies appropriate for testing in such a model include direct treatment with PROK2 or PROK1 or its derivatives, agonists, conjugates or variants, or cell-based therapies utilizing PROK2 or PROK1 or its derivatives, agonists, conjugates or variants. The latter method, commonly referred to as adoptive immunotherapy, would involve treatment of the animal with immune system components (i.e. lymphocytes, NK cells, dendritic cells or bone marrow, and the like) and may include ex vivo incubation of cells with PROK2 or PROK1 with or without other immunomodulatory agents described herein or known in the art.

Another syngeneic mouse tumor cell line can used to test the anti-cancer efficacy of PROK2 or PROK1 and to identify the immune (effector) cell population responsible for mediating this effect. EG.7ova is a thymoma cell line that has been modified (transfected) to express ovalbumin, an antigen foreign to the host. Mice bearing a transgenic T cell receptor specific for EG.7ova are available (OT-I transgenics, Jackson Laboratory). CD8 T cells isolated from these animals (OT-I T cells) have been demonstrated to kill EG.7 cells in vitro and to promote rejection of the tumor in vivo. EG.7ova cells can be passaged in culture and about 1,000,000 cells injected intraperitoneal into C57BL6 mice. Multiple tumor sites are established and grow within the peritoneal cavity. Therapies designed to kill or reduce the growth of tumor cells using PROK2 or PROK1 or its derivatives, agonists, conjugates or variants can be tested by administration of compounds to mice bearing the tumor cells. OT-I T cells can be administered to the mice to determine if their activity is enhanced in the presence of PROK2 or PROK1. Efficacy of treatment is measured and statistically evaluated by time of survival in the treated populations. Therapeutic strategies appropriate for testing in such models include direct treatment with PROK2 or PROK1 or its derivatives, agonists, conjugates or variants, or cell-based therapies utilizing PROK2 or PROK1 or its derivatives, agonists, conjugates or variants. Ex vivo treatment of cytotoxic T-lymphocytes (CTL) could also be used to test the PROK2 or PROK1 in the cell-based strategy.

Analysis of PROK2 or PROK1 efficacy for treating certain specific types of cancers are preferably made using animals that have been shown to correlate to other mammalian disease, particularly human disease. After PROK2 or PROK1 is administered in these models evaluation of the effects on the cancerous cells or tumors is made. Xenografts are used for most preclinical work, using immunodeficient mice. For example, a syngeneic mouse model for ovarian carcinoma utilizes a C57BL6 murine ovarian carcinoma cell line stably overexpressing VEGF16 isoform and enhanced green fluorescent protein (Zhang et al., Am. J. Pathol. 161:2295-2309, 2002). Renal cell carcinoma mouse models using Renca cell injections have been shown to establish renal cell metastatic tumors that are responsive to treatment with immunotherapeutics such as IL-12 and IL-2 (Wigginton et al., J. of Nat. Cancer Inst. 88:38-43, 1996). A colorectal carcinoma mouse model has been established by implanting mouse colon tumor MC-26 cells into the splenic subcapsule of BALB/c mice (Yao et al., Cancer Res. 63 (3):586-586-592, 2003). An immunotherapeutic-responsive mouse model for breast cancer has been developed using a mouse that spontaneously develops tumors in the mammary gland and demonstrates peripheral and central tolerance to MUC1 (Mukherjee et al., J. Immunotherapy 26:47-42, 2003). To test the efficacy of PROK2 or PROK1 in prostate cancer, animal models that closely mimic human disease have been developed. A transgenic adenocarcinoma of the mouse prostate model (TRAMP) is the most commonly used syngeneic model (Kaplan-Lefko et al., Prostate 55 (3):219-237, 2003; Kwon et al., PNAS 96:15074-15079, 1999; Arap et al., PNAS 99:1527-1531, 2002).

The angiogenic potential of the PROK2 proteins of the present invention can also measured in a murine model where a diffusion chamber is subcutaneously implanted into the mid back of a mouse. To prepare the diffusion chambers, approximately 20 membranes (Millipore, Danvers, Mass.; Catalogue No. HAWP 013 00) are removed from the holder and placed onto a water-dampened 4×4 gauze pad in a Petri dish. The membranes need to be wetted so they can swell and become larger than the Plexiglas ring. After approximately 10 minutes on the dampened gauze the membranes are ready for use. A Plexiglas ring with 0.59 mm hole (Millipore, Danvers, Mass.; Catalogue No. PR00 014 01) is placed on a Petri dish and via a Icc syringe with an attached 26G needle; MF cement (Millipore, Danvers, Mass.; Catalogue No. SD1M057E0) is distributed completely around one side of the Plexiglas ring. Using a pair of forceps, a membrane is picked up, touched to a dry gauze pad to wick off any excess fluid and then placed in contact with the cement on the Plexiglas ring. The membrane is pressed between two fingers to make good contact with the cement and set aside to dry. After a minimum of approximately 10 minutes, this same procedure is repeated to place another membrane on the other side of this Plexiglas ring. The completed rings are allowed to completely dry, usually 3-4 hours and then sealed in a Petri dish for sterilization. Sterilization is performed by placing the sealed Petri dish with the completed discs under an Ultraviolet light for 1-2 hours.

To load and implant the chambers, under sterile conditions, the Petri dish containing the discs is opened and a disc removed. Via the hole in the side of the Plexiglas ring, a 23G needle is inserted and approximately 200 μL of a solution containing cells or test material is injected. The needle is removed and the hole plugged with a short piece of nylon rod (included with the Plexiglas rings). The filled chamber is then ready for subcutaneous implantation. The mouse into which the chamber is to be placed is anesthetized with isoflurane inhalation anesthesia. While under anesthesia, the mouse is placed in ventral recumbency, the mid to lower dorsal skin scrubbed with a Povidone Iodine soap, wiped dry and finally prepped with a Povidone Iodine prep solution. Using aseptic technique, a 12-15 mm skin incision is created in the mid-back with a blunt scissors. Via blunt dissection, a pocket is created extending from the incision caudal to the base of the tail. Into this pocket, the chamber is inserted and advanced toward the tail base. The skin incision is closed with 2-3 skin staples.

The effect of PROK2 monoclonal antibodies on B-cell-derived tumors in vivo can be measured as follows. Administration of PROK2 is by constant infusion via mini-osmotic pumps resulting in steady state serum concentrations proportional to the concentration of the PROK2 contained in the pump. 0.22 ml of human PROK2 contained in phosphate buffered saline (pH 6.0) at a concentration of 2 mg/ml or 0.2 mg/ml is loaded under sterile conditions into Alzet mini-osmotic pumps (model 2004; Alza corporation Palo Alto, Calif.). Pumps are implanted subcutaneously in mice through a 1 cm incision in the dorsal skin, and the skin is closed with sterile wound closures. These pumps are designed to deliver their contents at a rate of 0.25 μl per hour over a period of 28 days. This method of administration can result in significant increase in tumor progression in mice injected with tumor cells (below).

The effects of PROK2 antagonists are measured in vivo using a mouse tumor xenograft model described herein. The xenograft models tested are human lymphoblastoid cell line IM-9 (ATCC No. CRL159). C.B-17 SCID mice (female C.B-17/IcrHsd-scid; Harlan, Indianapolis, Ind.) are divided into 4 groups. On day 0, IM-9 cells (ATCC No. CRL159) are harvested from culture and injected intravenously, via the tail vein, to all mice (about 1,000,000 cells per mouse). On day 1, mini-osmotic pumps containing test article or control article are implanted subcutaneously in the mice. Mice are divided into and are treated with increasing concentrations of PROK2 and the PROK2 monoclonal antibody. A reduction in the effects of the B-cell tumor cells in vivo, by the PROK2 monoclonal antibody will indicate increased survival.

The anti-tumor effects of anti-PROK antagonists can be measure in the in B16-F10 Melanoma and EG.7 Thymoma models as described herein. Briefly, mice (female, C57B16, 9 weeks old; Charles River Labs, Kingston, N.Y.) are divided into three groups. On day 0, B 16-F 10 melanoma cells (ATCC No. CRL-6475) are harvested from culture and injected intravenously, via the tail vein, to all mice (about 100,000 cells per mouse). Mice are then treated with the test article or associated vehicle by intraperitoneal injection of 0.1 ml of the indicated solution. Mice in the first group (n=24) are treated with vehicle (PBS pH 6.0), which is injected on day 0, 2, 4, 6, and 8. Mice in the second group (n=24) are treated with murine PROK2. Mice in the third group (n=12) are treated with a PROK2 monoclonal antibody. All of the mice are sacrificed on day 18, and lungs are collected for quantitation of tumor. Foci of tumor growth greater than 0.5 mm in diameter are counted on all surfaces of each lung lobe. Effect of a PROK antagonist is measured by a reduction in number of tumor foci present on lungs of the monoclonal antibody treated group as compared to mice treated with vehicle. The monoclonal antibodies of the present invention can either slow the growth of the B 16 melanoma tumors or enhance the ability of the immune system to destroy the tumor cells. The effects of the treatment on tumor cells may mediated through cells of the immune system.

In a similar model, mice (female, C57B16, 9 weeks old; Charles River Labs, Kingston, N.Y.) are divided into three groups. On day 0, EG.7 cells (ATCC No. CRL-2113) are harvested from culture and 1, 000, 000 cells are injected intraperitoneal in all mice. Mice are then treated with the test article or associated vehicle by intraperitoneal injection of 0.1 mL of the indicated solution. Mice in the first group (n=6) are treated with vehicle (PBS pH 6.0), which is injected on day 0, 2, 4, and 6. Mice in the second group (n=6) are treated with PROK2. Mice in the third group (n=6) are treated with a PROK2 monoclonal antibody. Effects of the monoclonal antibodies will be judged by an increased survival time compared to mice treated with vehicle.

The effect of a PROK antagonist on EG.7 thymoma growth can be measured in vivo. Cytotoxic T lymphocytes (CTL) recognize infected and transformed cells by virtue of the display of viral and tumor antigens on the cell surface. Effective anti-tumor responses require the stimulation and expansion of antigen specific CTL clones. This process requires the interaction of several cell types in addition to CTL and usually results in the establishment of immunologic memory. The EG-7 tumor cell line is transfected with chicken ovalbumin and thereby expresses a well characterized T cell antigen, an ova peptide (SEQ ID NO:17) presented in H-2 Kb. OT-I T cells (Example 21) kill EG7 tumor cells in vitro and in vivo. (Shrikant, P and Mescher, M, J. Immunology 162:2858-2866, 1999). Mice (female, C57B16, 9 weeks old; Charles River Labs, Kingston, N.Y.) are divided into three groups. On day 0, EG.7 cells (ATCC No. CRL-2113) are harvested from culture and 1,000, 000 cells are injected intraperitoneal in all mice. Mice are then treated with the test article or associated vehicle by intraperitoneal injection of 0.1 ml of the indicated solution. Mice in the first group (n=6) are treated with vehicle (PBS pH 6.0), which is injected on day 0, 2, 4, and 6. Mice in the second group (n=6) are treated with PROK2. Mice in the third group (n=6) are treated with a PROK2 monoclonal antibody. Increased time of survival is the desired effect of treatment with the PROK antagonist.

The effects of PROK antagonists on B-cell lymphomas can also be measured in an in vivo assay. Human B-lymphoma cell lines are maintained in vitro by passage in growth medium. The cells are washed thoroughly in PBS to remove culture components. SCID Mice are injected with (typically) one million human lymphoma cells via the tail vein in a 100 microliter volume. (The optimal number of cell injected is determined empirically in a pilot study to yield tumor take consistently with desired kinetics.) PROK2 treatment is begun the next day by either subcutaneous. implantation of an ALZET® osmotic mini-pump (ALZET, Cupertino, Calif.) or by daily i.p injection of PROK2 or vehicle. Mice are monitored for survival and significant morbidity. Mice that lose greater than 20% of their initial body weight are sacrificed, as well as mice that exhibit substantial morbidity such as hind limb paralysis. Depending on the lymphoma cell line employed, the untreated mice typically die in 3 to 6 weeks. For B cell lymphomas that secrete IgG or IgM, the disease progression can also be monitored by weekly blood sampling and measuring serum human Immunoglobulin levels by ELISA.

A. PROK2 Dose Response/IM-9 Model

Mice are injected with 1×106 IM-9 cells, and 28 day osmotic mini pumps implanted the following day. The pumps are loaded with the following concentrations of PROK2 to deliver: 0, 0.12, 1.2 or 12 micrograms per day with 8 mice per dose group.

B. PROK2 NK Depletion/IM-9 Model

Mice are depleted of NK-cells by administering 5 doses of anti-asialo-GM-1 antibody every third day beginning 15 days prior to injection of tumor cells or left undepleted as controls. Group I of the depleted and undepleted mice are treated with vehicle only; Group II are treated with PROK2; and Group III are treated with a PROK2 monoclonal antibody.

C. Other Cell Lines Tested

The following additional cell lines are tested using the model shown for IM-9 cells: CESS cells in SCID mice; RAJI cell implanted tumors; mice with RAMOS cell implanted tumors; and mice with HS SULTAN cell implanted tumors.

The effects of PROK2 can be measured in a Mouse Syngeneic Ovarian Carcinoma Model. The effect of PROK2, or antagonists thereof, is tested for efficacy in ovarian carcinoma using a mouse syngeneic model as described in Zhang et al., Am. J. of Pathol. 161:2295-2309, 2002. Briefly, using retroviral transfection and fluorescence-activated cell sorting a C57BL6 murine ID8 ovarian carcinoma cell line is generated that stably overexpresses the murine VEGF 164 isoform and the enhanced green fluorescence protein (GFP). The retroviral construct containing VEGF164 and GFP cDNAs is transfected into BOSC23 cells. The cells are analyzed by FACS cell sorting and GFP high positive cells are identified.

The ID8 VEGF164/GFP transfected cells are cultured to subconfluence and prepared in a single-cell suspension in phosphate buffer saline (PBS) and cold MATRIGEL (BD Biosciences, Bedford, Mass.). Six to eight week old femal C57BL6 mice are injected subcutaneously in the flank at 5×106 cells or untransfected control cells. Alternatively, the mice can be injected intraperitoneally at 7×106 cells or control cells. Animals are either followed for survival or sacrificed eight weeks after inoculation and evaluated for tumor growth. Mice are treated with a PROK2 monoclonal antibody beginning 3-14 days following tumor implantation, or when tumor engraftment and growth rate is established.

The effect of PROK2 can be measured in a in a mouse RENCA model. The efficacy of PROK2 in a renal cell carcinoma model can be evaluated using BALB/c mice that have been injected with RENCA cells, a mouse renal adenocarcinoma of spontaneous origin, essentially as described in Wigginton et al., J. Nat. Cancer Instit. 88:38-43, 1996.

Briefly, BALB/c mice between eight and ten weeks are injected with RENCA cells R 1×105 cells into the kidney capsule of the mice. Twelve days after tumor cell implantation, the mice are nepharectomized to remove primary tumors. The mice are allowed to recover from surgery, prior to administration of a PROK2 monoclonal antibody. Mice are treated beginning 3-14 days following tumor implantation, or when tumor engraftment and growth rate is established. Treatment will be administered on a daily basis for 5-14 days, and may be continued thereafter if no evidence of neutralizing antibody formation is seen. Alternatively, RENCA cells may be introduced by subcutaneous (5×10e5 cells) or intravenous (1×10e5 cells) injection. The mice are evaluated for tumor response as compared to untreated mice. Survival is compared using a Kaplan-Meier method, as well as tumor volume being evaluated.

The effects of PROK antagonists can be measured in a mouse colorectal tumor model. The effects of PROK2 in a colorectal mouse model are tested as described in Yao et al., Cancer Res. 63:586-592, 2003. In this model, MC-26 mouse colon tumor cells are implanted into the splenic subcapsul of BALB/c mice. After 14 days, the treated mice are administered a PROK2 monoclonal antibody. Mice are treated beginning 3-14 days following tumor implantation, or when tumor engraftment and growth rate is established. Treatment is administered on a daily basis for 5-14 days, and may be continued thereafter if no evidence of neutralizing antibody formation is seen. The efficacy of PROK antagonist in prolonging survival or promoting a tumor response is evaluated using standard techniques described herein.

The efficacy of PROK2 in a mouse pancreatic cancer model is evaluated using the protocol developed by Mukherjee et al., J. Immunol. 165:3451-3460, 2000. Briefly, MUC1 transgenic (MUC1.Tg) mice are bred with oncogene-expressing mice that spontaneously develop tumors of the pancreas (ET mice) designated as MET. MUC1.Tg mice. ET mice express the first 127 aa of SV40 large T Ag under the control of the rat elastase promoter. Fifty percent of the animals develop life-threatening pancreatic tumors by about 21 wk of age. Cells are routinely tested by flow cytometry for the presence of MUC1. All mice are on the C57BL/6 background. Animals are sacrificed and characterized at 3-wk intervals from 3 to 24 wk. Mice are carefully observed for signs of ill-health, including lethargy, abdominal distention, failure to eat or drink, marked weight loss, pale feces, and hunched posture.

The entire pancreas is dissected free of fat and lymph nodes, weighed, and spread on bibulus paper for photography. Nodules are counted, and the pancreas is fixed in methacam, processed for microscopy by conventional methods, step sectioned at 5 μm (about 10 sections per mouse pancreas), stained with hematoxylin and eosin, and examined by light microscopy. Tumors are obtained from MET mice at various time points during tumor progression, fixed in methacarn (60% methanol, 30% chloroform, 10% glacial acetic acid), embedded in paraffin, and sectioned for immunohistochemical analysis. MUC1 antibodies used are CT1, a rabbit polyclonal Ab that recognizes mouse and human cytoplasmic tail region of MUC1, HMFG-2, BC2, and SM-3, which have epitopes in the TR domain of MUC1.

Determination of CTL activity is performed using a standard 51Cr release method after a 6-day in vitro peptide stimulation without additional added cytokines. Splenocytes from individual MET mice are harvested by passing through a nylon mesh followed by lysis of RBC.

Single cells from spleens of MET mice are analyzed by two-color immunofluorescence for alterations in lymphocyte subpopulations: CD3, CD4, CD8, Fas, FasL, CD11c, and MHC class I and II. Intracellular cytokine levels are determined after cells are stimulated with MUC1 peptide (10 μg/ml for 6 days) and treated with brefeldin-A (also called Golgi-Stop; PharMingen) as directed by the manufacturer's recommendation (4 μl/1.2×107 cells/6 ml for 3 h at 37° C. before staining). Cells are permeabilized using the PharMingen permeabilization kit and stained for intracellular IFN-, IL-2, IL-4, and IL-5 as described by PharMingen. All fluorescently labeled Abs are purchased from PharMingen. Flow cytometric analysis is done on Becton Dickinson FACscan using the CellQuest program (Becton Dickinson, Mountain View, Calif.). Mice are treated with a PROK2 monoclonal antibody beginning 3-14 days following tumor implantation, or when tumor engraftment and growth rate is established. Treatment is administered on a daily basis for 5-14 days, and may be continued thereafter if no evidence of neutralizing antibody formation is seen.

The effect of a PROK2 antagonist in a murine model for breast cancer is made using a syngeneic model as described in Colombo et al., Cancer Research 62:941-946, 2002. Briefly, TS/A cells which are a spontaneous mammary carcinoma for BALB/C mice. The cells are cultured for approximately one week to select for clones. The selected TS/A cells are grown and used to challenge CD-1 nu/nu BR mice (Charles River Laboratories) by injected 2×102 TS/A cells subcutaneously into the flank of the mouse.

Mice are treated with a PROK2 monoclonal antibody beginning 3-14 days following tumor implantation, or when tumor engraftment and growth rate is established. Treatment is administered on a daily basis for 5-14 days, and may be continued thereafter if no evidence of neutralizing antibody formation is seen. The tumors are excised after sacrificing the animals and analyzed for volume and using histochemistry and immunohistochemistry.

The effects of PROK2 antagonists on tumor response are evaluated in murine prostate cancer model, using a model similar to that described in Kwon et al., PNAS 96:15074-15079, 1999. In this model, there is a metastatic outgrowth of transgenic adenocarcinoma of mouse prostate (TRAMP) derived prostate cancer cell line TRAMP-C2, which are implanted in C57BL/6 mice. Metastatic relapse is reliable, occurring primarily in the draining lymph nodes in close proximity to the primary tumor.

Briefly, the C2 cell line used is an early passage line derived from the TRAMP mouse that spontaneously develops autochthonous tumors attributable to prostate-restricted SV40 antigen expression. The cells are cultured and injected subcutaneously into the C57BL/6 mice at 2.5−5×106 cells/0.1 ml media. Mice are treated with a PROK2 monoclonal antibody beginning 3-14 days following tumor implantation, or when tumor engraftment and growth rate is established. Treatment is administered on a daily basis for 5-14 days, and may be continued thereafter if no evidence of neutralizing antibody formation is seen. The tumors are excised after sacrificing the animals and analyzed for volume and using histochemistry and immunohistochemistry.

In these models, the effects of the monoclonal antibodies, fragments, or variants thereof can be measured for inhibition, reduction, or delay on progression of the tumor.

### 8. Dosage and Administration of PROK Antagonists

Generally, the dosage of administered antibodies or antagonists will vary depending upon such factors as the patient's age, weight, height, sex, general medical condition and previous medical history. Typically, it is desirable to provide the recipient with a dosage of a molecule having anti-PROK activity, which is in the range of from about 1 pg/kg to 10 mg/kg (amount of agent/body weight of patient), although a lower or higher dosage also may be administered as circumstances dictate.

Administration of a molecule having anti-PROK activity to a subject can be intravenous, intraarterial, intraperitoneal, intramuscular, subcutaneous, intrapleural, intrathecal, by perfusion through a regional catheter, inhalation, as a suppository, or by direct intralesional injection. When administering therapeutic proteins by injection, the administration may be by continuous infusion or by single or multiple boluses. Alternatively, anti-PROK polypeptides, such as anti-PROK2, anti-PROK1, as well as fragments, variants and/or chimeras thereof, can be administered as a controlled release formulation.

Additional routes of administration include oral, dermal, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Illum, Adv. Drug Deliv. Rev. 35:199 (1999)). Dry or liquid particles comprising such as anti-PROK2, anti-PROK1, as well as fragments, variants and/or chimeras thereof, can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850 (1995)). Transdermal delivery using electroporation provides another means to administer such as PROK2, PROK1, as well as agonists, fragments, variants and/or chimeras thereof, (Potts et al., Pharm. Biotechnol. 10:213 (1997)).

PROK antagonists can also be applied topically as, for example, liposomal preparations, gels, salves, as a component of a glue, prosthesis, or bandage, and the like.

A pharmaceutical composition comprising molecules having PROK2 or PROK1 antagonist activity can be furnished in liquid form, in an aerosol, or in solid form. Proteins having PROK2 or PROK1 antagonist activity can be administered as a conjugate with a pharmaceutically acceptable water-soluble polymer moiety. As an illustration, a PROK2 antagonist-polyethylene glycol conjugate is useful to increase the circulating half-life of the interferon, and to reduce the immunogenicity of the polypeptide. Liquid forms, including liposome-encapsulated formulations, are illustrated by injectable solutions and oral suspensions. Exemplary solid forms include capsules, tablets, and controlled-release forms, such as a miniosmotic pump or an implant. Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

The anti-PROK antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

A pharmaceutical composition comprising a protein, polypeptide, or peptide having PROK2 or PROK1 antagonist activity can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

For purposes of therapy, molecules having anti-PROK2 or anti-PROK1 activity and a pharmaceutically acceptable carrier are administered to a patient in a therapeutically effective amount. A combination of a protein, polypeptide, or peptide having PROK activity and a pharmaceutically acceptable carrier is said to be administered in a “therapeutically effective amount” if the amount administered is physiologically significant. An agent is physiologically significant if its presence results in a detectable change in the physiology of a recipient patient.

For example, the present invention includes methods of increasing or decreasing gastrointestinal symptoms related to IBD and IBS, such as inflammation, contractility, gastric emptying, and/or intestinal transt, comprising the step of administering a composition comprising an anti-PROK, such as antagonists, antibodies, binding proteins, variants and fragments polypeptide, to the patient. In an in vivo approach, the composition is a pharmaceutical composition, administered in a therapeutically effective amount to a mammalian subject. Additionally, the anti-PROK antibodies of the present invention can be used to reduce, inhibit or delay progression of tumor, angiogenesis and vascularization.

A pharmaceutical composition comprising molecules having anti-PROK activity can be furnished in liquid form, or in solid form. Liquid forms, including liposome-encapsulated formulations, are illustrated by injectable solutions and oral suspensions. Exemplary solid forms include capsules, tablets, and controlled-release forms, such as a miniosmotic pump or an implant. Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

Anti-PROK2 or anti-PROK1 pharmaceutical compositions may be supplied as a kit comprising a container that comprises a PROK2 or PROK1 antagonist (e.g., an anti-PROK2 or PROK1 antibody or antibody fragment). For example, anti-PROK2 or anti-PROK1 can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic polypeptide. Such a kit may further comprise written information on indications and usage of the pharmaceutical composition.

Administration of anti-PROK2 and anti-PROK1 monoclonal antibodies of using the methods of the present invention will result in a tumor response. While each protocol may define tumor response assessments differently, exemplary guidelines can be found in Clinical Research Associates Manual, Southwest Oncology Group, CRAB, Seattle, Wash., Oct. 6, 1998, updated August 1999. According to the CRA Manual (see, chapter 7 “Response Assessment”), tumor response means a reduction or elimination of all measurable lesions or metastases. Disease is generally considered measurable if it comprises bidimensionally measurable lesions with clearly defined margins by medical photograph or X-ray, computerized axial tomography (CT), magnetic resonance imaging (MRI), or palpation. Evaluable disease means the disease comprises unidimensionally measurable lesions, masses with margins not clearly defined, lesion with both diameters less than 0.5 cm, lesions on scan with either diameter smaller than the distance between cuts, palpable lesions with diameter less than 2 cm, or bone disease. Non-evaluable disease includes pleural effusions, ascites, and disease documented by indirect evidence. Previously radiated lesions which have not progressed are also generally considered non-evaluable.

The criteria for objective status are required for protocols to access solid tumor response. A representative criteria includes the following: (1) Complete Response (CR) defined as complete disappearance of all measurable and evaluable disease. No new lesions. No disease related symptoms. No evidence of non-evaluable disease; (2) Partial Response (PR) defined as greater than or equal to 50% decrease from baseline in the sum of products of perpendicular diameters of all measurable lesions. No progression of evaluable disease. No new lesions. Applies to patients with at least one measurable lesion; (3) Progression defined as 50% or an increase of 10 cm² in the sum of products of measurable lesions over the smallest sum observed using same techniques as baseline, or clear worsening of any evaluable disease, or reappearance of any lesion which had disappeared, or appearance of any new lesion, or failure to return for evaluation due to death or deteriorating condition (unless unrelated to this cancer); (4) Stable or No Response defined as not qualifying for CR, PR, or Progression. (See, Clinical Research Associates Manual, supra.)

### 9. Detection of PROK Gene Expression with Nucleic Acid Probes

Nucleic acid molecules can be used to detect the expression of a PROK2 or PROK1 gene in a biological sample, including diagnostic staging in cancer, tumors, angiogenesis, and inflammation associated cancer cells and tissues. Such probe molecules include double-stranded nucleic acid molecules comprising the nucleotide sequence of SEQ ID NO:1, or a fragment thereof, as well as single-stranded nucleic acid molecules having the complement of the nucleotide sequence of SEQ ID NO:1, or a fragment thereof. Probe molecules may be DNA, RNA, oligonucleotides, and the like.

Illustrative probes comprise a portion of the nucleotide sequence of nucleotides 66 to 161 of SEQ ID NO:1, the nucleotide sequence of nucleotides 288 to 389 of SEQ ID NO:1, or the complement of such nucleotide sequences. An additional example of a suitable probe is a probe consisting of nucleotides 354 to 382 of SEQ ID NO:1, or a portion thereof. As used herein, the term “portion” refers to at least eight nucleotides to at least 20 or more nucleotides.

For example, nucleic acid molecules comprising a portion of the nucleotide sequence of SEQ ID NO:1 or of SEQ ID NO:4, can be used to detect activated neutrophils. Such molecules can also be used to identity therapeutic or prophylactic agents that modulate the response of a neutrophil to a pathogen.

In a basic detection assay, a single-stranded probe molecule is incubated with RNA, isolated from a biological sample, under conditions of temperature and ionic strength that promote base pairing between the probe and target PROK2 RNA species. After separating unbound probe from hybridized molecules, the amount of hybrids is detected.

Well-established hybridization methods of RNA detection include northern analysis and dot/slot blot hybridization (see, for example, Ausubel (1995) at pages 4-1 to 4-27, and Wu et al. (eds.), “Analysis of Gene Expression at the RNA Level,” in Methods in Gene Biotechnology, pages 225-239 (CRC Press, Inc. 1997)). Nucleic acid probes can be detectably labeled with radioisotopes such as ³²P or ³⁵S. Alternatively, PROK RNA can be detected with a nonradioactive hybridization method (see, for example, Isaac (ed.), Protocols for Nucleic Acid Analysis by Nonradioactive Probes (Humana Press, Inc. 1993)). Typically, nonradioactive detection is achieved by enzymatic conversion of chromogenic or chemiluminescent substrates. Illustrative nonradioactive moieties include biotin, fluorescein, and digoxigenin.

PROK2 oligonucleotide probes are also useful for in vivo diagnosis. As an illustration, ¹⁸F-labeled oligonucleotides can be administered to a subject and visualized by positron emission tomography (Tavitian et al., Nature Medicine 4:467 (1998)).

Numerous diagnostic procedures take advantage of the polymerase chain reaction (PCR) to increase sensitivity of detection methods. Standard techniques for performing PCR are well-known (see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

One variation of PCR for diagnostic assays is reverse transcriptase-PCR(RT-PCR). In the RT-PCR technique, RNA is isolated from a biological sample, reverse transcribed to cDNA, and the cDNA is incubated with PROK2 primers (see, for example, Wu et al. (eds.), “Rapid Isolation of Specific cDNAs or Genes by PCR,” in Methods in Gene Biotechnology, pages 15-28 (CRC Press, Inc. 1997)). PCR is then performed and the products are analyzed using standard techniques.

As an illustration, RNA is isolated from biological sample using, for example, the guanidinium-thiocyanate cell lysis procedure described above. Alternatively, a solid-phase technique can be used to isolate mRNA from a cell lysate. A reverse transcription reaction can be primed with the isolated RNA using random oligonucleotides, short homopolymers of dT, or PROK2 anti-sense oligomers. Oligo-dT primers offer the advantage that various mRNA nucleotide sequences are amplified that can provide control target sequences. PROK2 sequences are amplified by the polymerase chain reaction using two flanking oligonucleotide primers that are typically 20 bases in length.

PCR amplification products can be detected using a variety of approaches. For example, PCR products can be fractionated by gel electrophoresis, and visualized by ethidium bromide staining. Alternatively, fractionated PCR products can be transferred to a membrane, hybridized with a detectably-labeled PROK2 probe, and examined by autoradiography. Additional alternative approaches include the use of digoxigenin-labeled deoxyribonucleic acid triphosphates to provide chemiluminescence detection, and the C-TRAK colorimetric assay.

Another approach for detection of PROK expression is cycling probe technology (CPT), in which a single-stranded DNA target binds with an excess of DNA-RNA-DNA chimeric probe to form a complex, the RNA portion is cleaved with RNAase H, and the presence of cleaved chimeric probe is detected (see, for example, Beggs et al., J. Clin. Microbiol. 34:2985 (1996), Bekkaoui et al., Biotechniqes 20:240 (1996)). Alternative methods for detection of PROK2 sequences can utilize approaches such as nucleic acid sequence-based amplification (NASBA), cooperative amplification of templates by cross-hybridization (CATCH), and the ligase chain reaction (LCR) (see, for example, Marshall et al., U.S. Pat. No. 5,686,272 (1997), Dyer et al., J. Virol. Methods 60:161 (1996), Ehricht et al., Eur. J. Biochem. 243:358 (1997), and Chadwick et al., J. Virol. Methods 70:59 (1998)). Other standard methods are known to those of skill in the art.

PROK2 probes and primers can also be used to detect and to localize PROK2 gene expression in tissue samples. Methods for such in situ hybridization are well-known to those of skill in the art (see, for example, Choo (ed.), In Situ Hybridization Protocols (Humana Press, Inc. 1994), Wu et al. (eds.), “Analysis of Cellular DNA or Abundance of mRNA by Radioactive In Situ Hybridization (RISH),” in Methods in Gene Biotechnology, pages 259-278 (CRC Press, Inc. 1997), and Wu et al. (eds.), “Localization of DNA or Abundance of mRNA by Fluorescence In Situ Hybridization (RISH),” in Methods in Gene Biotechnology, pages 279-289 (CRC Press, Inc. 1997)). Various additional diagnostic approaches are well-known to those of skill in the art (see, for example, Mathew (ed.), Protocols in Human Molecular Genetics (Humana Press, Inc. 1991), Coleman and Tsongalis, Molecular Diagnostics (Humana Press, Inc. 1996), and Elles, Molecular Diagnosis of Genetic Diseases (Humana Press, Inc., 1996)).

Example 14, below, shows a method that can be used to detect and monitor IBD in patient samples. As discussed above, biological samples, including biopsy specimens can be screened for the presence of the polynucleotide sequences of SEQ ID NO:1 or SEQ ID NO:4, or a fragment thereof, to determine if PROK2 or PROK1 is upregulated in the sample.

### 10. Detection of PROK2 Protein with Anti-PROK2 Antibodies

The present invention contemplates the use of anti-PROK2 antibodies to screen biological samples in vitro for the presence of PROK2, and particularly for the upregulation of PROK2. In one type of in vitro assay, anti-PROK2 antibodies are used in liquid phase. For example, the presence of PROK2 in a biological sample can be tested by mixing the biological sample with a trace amount of labeled PROK2 and an anti-PROK2 antibody under conditions that promote binding between PROK2 and its antibody. Complexes of PROK2 and anti-PROK2 in the sample can be separated from the reaction mixture by contacting the complex with an immobilized protein which binds with the antibody, such as an Fc antibody or Staphylococcus protein A. The concentration of PROK2 in the biological sample will be inversely proportional to the amount of labeled PROK2 bound to the antibody and directly related to the amount of free-labeled PROK2. Anti-PROK1 antibodies can be used in the same or a similar fashion.

Alternatively, in vitro assays can be performed in which anti-PROK2 antibody is bound to a solid-phase carrier. For example, antibody can be attached to a polymer, such as aminodextran, in order to link the antibody to an insoluble support such as a polymer-coated bead, a plate or a tube. Other suitable in vitro assays will be readily apparent to those of skill in the art.

In another approach, anti-PROK2 antibodies can be used to detect PROK2 in tissue sections prepared from a biopsy specimen. Such immunochemical detection can be used to determine the relative abundance of PROK2 and to determine the distribution of PROK2 in the examined tissue. General immunochemistry techniques are well established (see, for example, Ponder, “Cell Marking Techniques and Their Application,” in Mammalian Development: A Practical Approach, Monk (ed.), pages 115-38 (IRL Press 1987), Coligan at pages 5.8.1-5.8.8, Ausubel (1995) at pages 14.6.1 to 14.6.13 (Wiley Interscience 1990), and Manson (ed.), Methods In Molecular Biology, Vol. 10: Immunochemical Protocols (The Humana Press, Inc. 1992)).

Immunochemical detection can be performed by contacting a biological sample with an anti-PROK2 antibody, and then contacting the biological sample with a detectably labeled molecule that binds to the antibody. For example, the detectably labeled molecule can comprise an antibody moiety that binds to anti-PROK2 antibody. Alternatively, the anti-PROK2 antibody can be conjugated with avidin/streptavidin (or biotin) and the detectably labeled molecule can comprise biotin (or avidin/streptavidin). Numerous variations of this basic technique are well-known to those of skill in the art.

Alternatively, an anti-PROK2 antibody can be conjugated with a detectable label to form an anti-PROK2 immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below.

The detectable label can be a radioisotope that is detected by autoradiography. Isotopes that are particularly useful for the purpose of the present invention are ³H, ¹²⁵I, ¹³¹I, ³⁵S and ¹⁴C.

Anti-PROK2 immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine.

Alternatively, anti-PROK2 immunoconjugates can be detectably labeled by coupling an antibody component to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label anti-PROK2 immunoconjugates of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-PROK2 immunoconjugates can be detectably labeled by linking an anti-PROK2 antibody component to an enzyme. When the anti-PROK2-enzyme conjugate is incubated in the presence of the appropriate substrate, the enzyme moiety reacts with the substrate to produce a chemical moiety, which can be detected, for example, by spectrophotometric, fluorometric or visual means. Examples of enzymes that can be used to detectably label polyspecific immunoconjugates include β-galactosidase, glucose oxidase, peroxidase and alkaline phosphatase.

Those of skill in the art will know of other suitable labels, which can be employed in accordance with the present invention. The binding of marker moieties to anti-PROK2 antibodies can be accomplished using standard techniques known to the art. Typical methodology in this regard is described by Kennedy et al., Clin. Chim. Acta 70:1 (1976), Schurs et al., Clin. Chim. Acta 81:1 (1977), Shih et al., Int'l J. Cancer 46:1101 (1990), Stein et al., Cancer Res. 50:1330 (1990), and Coligan, supra.

Moreover, the convenience and versatility of immunochemical detection can be enhanced by using anti-PROK2 antibodies that have been conjugated with avidin, streptavidin, and biotin (see, for example, Wilchek et al. (eds.), “Avidin-Biotin Technology,” Methods In Enzymology, Vol. 184 (Academic Press 1990), and Bayer et al., “Immunochemical Applications of Avidin-Biotin Technology,” in Methods In Molecular Biology, Vol. 10, Manson (ed.), pages 149-162 (The Humana Press, Inc. 1992).

Methods for performing immunoassays are well-established. See, for example, Cook and Self, “Monoclonal Antibodies in Diagnostic Immunoassays,” in Monoclonal Antibodies: Production, Engineering, and Clinical Application, Ritter and Ladyman (eds.), pages 180-208, (Cambridge University Press, 1995), Perry, “The Role of Monoclonal Antibodies in the Advancement of Immunoassay Technology,” in Monoclonal Antibodies: Principles and Applications, Birch and Lennox (eds.), pages 107-120 (Wiley-Liss, Inc. 1995), and Diamandis, Immunoassay (Academic Press, Inc. 1996).

In a related approach, biotin- or FITC-labeled PROK2 can be used to identify cells that bind PROK2. Such can binding can be detected, for example, using flow cytometry.

The present invention also contemplates kits for performing an immunological diagnostic assay for PROK2 gene expression. Such kits comprise at least one container comprising an anti-PROK2 antibody, or antibody fragment. A kit may also comprise a second container comprising one or more reagents capable of indicating the presence of PROK2 antibody or antibody fragments. Examples of such indicator reagents include detectable labels such as a radioactive label, a fluorescent label, a chemiluminescent label, an enzyme label, a bioluminescent label, colloidal gold, and the like. A kit may also comprise a means for conveying to the user that PROK2 antibodies or antibody fragments are used to detect PROK2 protein. For example, written instructions may state that the enclosed antibody or antibody fragment can be used to detect PROK2. The written material can be applied directly to a container, or the written material can be provided in the form of a packaging insert.

Diagnosis of IBS to date has been limited to using criteria that correlate with symptoms. For example, the major criteria include the Manning criteria and the Rome criteria. See Farhadi, A. et al., Expert Opin. Investig. Drugs 10(7): 1211-1222, 2001. The Manning criteria consider: 1) pain that is improved after bowel movement; 2) looser stool at the onset of pain; 3) more frequent stool at the onset of pain; and 4) visible bowel distension. The Rome criteria consider: 1) relief upon defacation; 2) onset associated with change in frequency of stool; and 3) onset associated with change in form (appearance) of stool. An improved method of detecting and monitoring IBS can be the use of anti-PROK antibodies, including anti-PROK2 and anti-PROK1 antibodies to screen biological samples from patients with IBS. Example 15, below, shows a method that can be used to detect and monitor IBD in patient samples. As discussed above, biological samples, including biopsy specimens can be screened for the presence of the polypeptide sequences of SEQ ID NO:2 or SEQ ID NO:5, or a fragment thereof, to determine if PROK2 or PROK1 is upregulated in the sample. As such PROK polypeptides and nucleic acids of the present invention can be used as a diagnostic marker for Irritable Bowel Syndrome.

The present invention, thus generally described, will be understood more readily by reference to the following examples, which are provided by way of illustration and are not intended to be limiting of the present invention. The examples describe studies using PROK2 protein produced in baculovirus with a C-terminal Glu-Glu tag, following the methods generally described above. PROK1 (“endocrine-gland-derived vascular endothelial growth factor”) protein was purchased from Peprotech, Inc. (Rocky Hill, N.J.).

The invention provides an antibody that specifically binds a polypeptide comprising the amino acid sequence of SEQ ID NO: 2, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859). Within an embodiment, the hybridoma is selected from: a) the hybridoma of clone designation number 279.124.1.4 (ATCC Patent Deposit Designation PTA-6857); b) the hybridoma of clone designation number 279.126.5.6.5 (ATCC Patent Deposit Designation PTA-6858); and c) the hybridoma of clone designation number 279.121.7.4 (ATCC Patent Deposit Designation PTA-6859). Within an embodiment the hybridoma is hybridoma of clone designation number 279.124.1.4 (ATCC Patent Deposit Designation PTA-6857). Within another embodiment, the hybridoma is hybridoma of clone designation number 279.126.5.6.5 (ATCC Patent Deposit Designation PTA-6858). Within another embodiment, the hybridoma is hybridoma of clone designation number 279.121.7.4 (ATCC Patent Deposit Designation PTA-6859). Within another embodiment, the hybridoma is hybridoma of clone designation number 279.111.5.2 (ATCC Patent Deposit Designation PTA-6856). Within another embodiment, the antibody is capable of binding the polypeptide as shown in SEQ ID NO: 5.

The invention provides a method of reducing, inhibiting or preventing angiogenesis comprising admixing an antibody with a polypeptide as shown in SEQ ID NO: 2, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide. Within an embodiment, the binding of the antibody to the polypeptide inhibits, reduces or prevents signal transduction by the polypeptide on its receptor. Within an embodiment, the antibody neutralizes the signal transduction. Within an embodiment, there is also an inhibition of chemokine release. Within an embodiment, the chemokine is GROα.

The invention provides a method of reducing, inhibiting or preventing angiogenesis comprising admixing an antibody with a polypeptide as shown in SEQ ID NO: 5, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide.

The invention provides a method of reducing, inhibiting or preventing tumor formation or tumor size comprising admixing an antibody with a polypeptide as shown in SEQ ID NO: 2, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide. Within an embodiment, the binding of the antibody to the polypeptide inhibits, reduces or prevents signal transduction by the polypeptide on its receptor. Within an embodiment, the antibody neutralizes the signal transduction. Within an embodiment, there is also an inhibition of chemokine release. Within an embodiment, the chemokine is GROα.

The invention provides a method of reducing, inhibiting or preventing tumor formation or tumor size comprising admixing an antibody with a polypeptide as shown in SEQ ID NO: 5, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide.

The invention provides a method of decreasing vascular leakage comprising admixing an antibody with a polypeptide as shown in SEQ ID NO: 2, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide. Within an embodiment, the binding of the antibody to the polypeptide inhibits, reduces or prevents signal transduction by the polypeptide on its receptor. Within an embodiment, the antibody neutralizes the signal transduction. Within an embodiment, there is also an inhibition of chemokine release. Within an embodiment, the chemokine is GROα.

The invention provides a method of decreasing vascular leakage comprising admixing an antibody with a polypeptide as shown in SEQ ID NO: 5, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide.

The invention provides a method of inhibiting, reducing or preventing metastasis formation comprising admixing an antibody with a polypeptide as shown in SEQ ID NO: 2, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide. Within an embodiment, the binding of the antibody to the polypeptide inhibits, reduces or prevents signal transduction by the polypeptide on its receptor. Within an embodiment, the antibody neutralizes the signal transduction. Within an embodiment, there is also an inhibition of chemokine release. Within an embodiment, the chemokine is GROA.

The invention provides a method of reducing, inhibiting or preventing metastasis formation or tumor size comprising admixing an antibody with a polypeptide as shown in SEQ ID NO: 5, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide.

The invention provides a method of inhibiting, reducing or preventing secretion of the polypeptide as shown by the amino acid sequence of SEQ ID NO: 2, comprising admixing an antibody with a polypeptide as shown in SEQ ID NO: 2, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide.

The invention provides a method of inhibiting, reducing, or delaying progression of inflammation comprising admixing an antibody with a polypeptide as shown in SEQ ID NO: 2, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide.

The invention provides a method of detecting a polypeptide comprising admixing the polypeptide with an antibody wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide. Within an embodiment, the polypeptide comprising the amino acid sequence of SEQ ID NO: 2 or SEQ ID NO: 5, or a fragment thereof. Within an embodiment, the polypeptide is detected in serum. Within an embodiment, the serum is from a patient with cancer.

The invention provides a method of inhibiting or reducing neutrophil infiltration comprising admixing an antibody with a polypeptide as shown in SEQ ID NO: 2, wherein the polypeptide is capable of binding the antibody produced by the hybridoma selected from: a) the hybridoma of clone designation number 279.111.5.2 (ATCC Patent Deposit Designation PTA-6856); b) the hybridoma of clone designation number 279.124.1.4 (ATCC Patent Deposit Designation PTA-6857); c) the hybridoma of clone designation number 279.126.5.6.5 (ATCC Patent Deposit Designation PTA-6858); and d) the hybridoma of clone designation number 279.121.7.4 (ATCC Patent Deposit Designation PTA-6859); and where in the antibody binds to the polypeptide.

The invention provides methods of reducing, limiting, inhibiting, and/or neutralizing the effects of PROK2, including antagonizing the effects of signal transduction caused by PROK2 on the GP37a or GP37b receptor. Such antagonistic effects will result in a reduction, limitation, neutralization or inhibition of angiogenesis, tumor formation, tumor size, metastaisi, vascular leakage, secretion of PROK2 from polymorphonuclear monocytes. Such antagonistic effects will be useful in a variety of cancers, such as colon cancer, breast cancer, renal cancer, neroblastoma, AML, solid tumors in general, and metastases. The antibodies produced by the deposited hybridomas described herein will be useful in treating these disorders as well as inflammation.

### 11. Examples

### Example 1

### Response of W12-22 Cells PROK2 and PROK1 Stimulation

Wky12-22 cells were derived from the medial layer of the thoracic aorta of Wistar-Kyoto rat pups, as described by Lemire et al., American Journal of Pathology 144:1068 (1994). These cells respond to both PROK2 and PROK1 in a reporter luciferase assay following transfection with NFkB/Ap-1 reporter construct. A control cell line, Wky3M-22, derived from the same tissue in adult rat did not signal. Activity was detected at concentrations ranging from 1-100 ng/ml of PROK2 or PROK1 (approximately 0.1 nM-10 nM). These data suggest that Wky12-22 cells carry the PROK2 receptor, and that PROK2 and PROK1 activate the NfKb/Ap-1 transcription factor.

In one experiment, Wky12-22 cells were loaded with the fluorescent dye Fura. The emission peak of Fura shifts when bound to calcium. Intracellular calcium release is detected by monitoring the wavelength shift. PROK2 induced intracellular calcium release at concentrations of 1-1000 ng/ml. PROK1 induced a similar response.

Extracellular signal-regulated kinase/mitogen-activated protein kinase (ERK-Map kinase) activity was measured in Wky12-22 cells in response to PROK2 treatment. Cells were incubated in PROK2 at concentrations ranging from 1 to 1,000 ng/ml for thirty minutes. Cells were fixed and stained for phosphorylated ERK-Map kinase using the Arrayscan, which measures the fluorescent intensities in the cytosol and the nucleus of the treated cell. The difference in fluorescence of the nucleus and the cytosol were quantified and plotted. PROK2 induced ERK-Map kinase activity with an EC₅₀ of 0.50 nM (approximately 5 ng/ml).

The binding of PROK2 to Wky12-22 cells was assessed using I¹²⁵-radiolabeled PROK2. Wky12-22 cells were seeded at low cell density and cultured for three to four days until they reached about 70% confluency. The cells were placed on ice, the medium was removed, and the monolayers were washed. The cells were incubated with increasing amounts of I¹²⁵-PROK2 in the absence (total binding) and presence (nonspecific binding) of a large excess of unlabeled PROK2. After various times at 4° C., the binding media were removed, the monolayers were washed, and the cells were solubilized with a small volume of 1.0 N NaOH. Cell associated radioactivity was determined in a gamma counter. The specific binding of I¹²⁵-PROK2 was calculated as the difference between the total and nonspecific values. The measured radioacitivity was normalized to cell number that was determined on a set of parallel cultures. Nonlinear regression using a two-site model was used to fit the binding data for determination of Kd and Bmax. The high affinity site exhibited a Kd of 1.5 nM and a Bmax of 350 fmol bound/10⁶ cells whereas the low affinity site showed a Kd of 31 nM with a Bmax of 1025 fmol bound/10⁶ cells.

The results of these studies show that a neonatal rat aortic cell expresses the PROK2 receptor while equivalent adult rat cells do not. This suggests that PROK2 is involved with heart development and vasculogenesis. PROK2 signals through NFkB/Ap1 and induces chemokine release only in the neonatal cells, suggesting that it may trigger a mitogenic response in fetal or neonatal heart. PROK2 may be a required factor necessary for the induction of vasculogenesis/angiogenesis in cardiac stem cells. PROK2 induces intracellular calcium release in the Wky12-22 cell line, an effect consistent with chemokine activity. Consistent with its mitogenic activity, PROK2 activates a mitogen activated protein kinase.

### Example 2

PROK2 and PROK1 Stimulate Chemokine Release In Vitro

Confluent Wky12-22 or Wky3M22 cells were incubated with varying concentrations of PROK2 for twenty-four hours. Conditioned media were collected and assayed for the chemokine CINC-1 using a commercially-available rat cytokine multiplex kit (Linco Research, Inc.; St. Charles, Mo.). CINC-1, thought to be equivalent to human growth-related oncogene-α (GRO-α), was detected at levels ranging from 1.8-5 ng/ml in cells treated with 0.1 to 100 ng/ml of PROK2 respectively. PROK1 induced an equivalent level of CINC-1 release from Wky12-22 cells. CINC-1 was not detected in either the control Wky3M-22 cell line derived from adult rat aorta, or non-treated controls.

### Example 3

PROK2 Induces a Chemotactic Response and Stimulates Chemokine Release and Neutrophil Infiltration In Vivo

Four groups of ten mice (BALB57/BL6 females at eight weeks of age) were either not treated, or injected with vehicle buffer control, 0.1 μg of PROK2 or 1 μg of PROK2. Four hours later, peritoneal lavage fluid was collected, concentrated, and the cell pellets were resuspended. The relative cell populations were enumerated using the Cell Dyne, and cytospins were prepared for CBC/diff counts. The non-treated and buffer control animals had approximately 2% neutrophils in their lavage fluid, while the 0.1 μg treated animals had approximately 30% neutrophils, indicating an approximate 15-fold increase in neutrophils in the peritoneum of the PROK2-treated animals. The 1 μg PROK2-treated animals had neutrophil levels consistent with the non-treated controls, suggesting a bi-phasic PROK2 response. In sum, PROK2 induced neutrophil infiltration into the peritoneum following intraperitoneal injection.

Murine KC, the ortholog of GROα in mice, was measured in serum and lavage fluids obtained from the four groups of mice using an ELISA kit (R&D Systems Inc.; MN). The 0.1 μg PROK2-treated (low dose) mice had approximately 45 picograms/ml KC in their peritoneal fluid, which was significantly higher than the non-treated controls, the vehicle controls, and the 1.0 μg PROK2-treated (high dose) mice.

Serum levels of KC in the 0.1 μg PROK2-treated mice were considerably higher than the non-treated, the 1.0 μg PROK2-treated, and the vehicle-treated mice. The 0.1 μg PROK2-treated mice had KC levels of approximately 185 picograms/ml, which is a six-fold increase.

TABLE 2
Murine KC in PROK2-treated mice following IP injection

| | Concentration of Murine KC | | | |
|---|---|---|---|---|
| | (picogram/ml) | | | |
| | Non-treated | Vehicle | 0.1 μg | 1.0 μg |
| | animals | Control | PROK2/animal | PROK2/animal |
| Lavage Fluid | 10 | 21 | 45 | 8 |
| Serum | 30 | 38 | 185 | 50 |

These results are consistent with the stimulation of chemokine release in vitro shown in Example 2. Furthermore these results correlate with the observed neutrophil infiltration in the peritoneum in the 0.1 μg PROK2-treated (low dose) mice.

### Example 4

### PROK2 Effect on Gastric Emptying

Seven mice received an intraperitoneal injection of approximately 200 μg of PROK2 (10 μg/g body weight) or vehicle control followed by 7.5 mg phenol red. Gastric function was measured by monitoring phenol red transport through the gut after twenty minutes. The general behavior of PROK2 treated animals was observed and was consistent with the behavior of the control animals. In the PROK2-treated mice, gastric transit time was reduced by approximately 50%.

These results show that, at high doses following intraperitoneal injection, PROK2 reduces gastric transit. PROK2 administration did not appear to have any immediate toxic effects. This reduction in transit may be the result of a massive muscle contraction at such high doses. PROK2 may well increase motility in vivo at low doses, and inhibit motility at high doses.

### Example 5

### Stimulation of Angiogenesis by PROK2 and PROK1

Thoracic aortas were removed from twelve-day, five-week, and three-month old Wistar rats. The tissues were flushed with Hanks basic salt solution to remove any blood cells and adventitial tissues were removed. Aortic rings were prepared and plated on Matrigel coated plates in serum free modified MCDB media from Clonetics plus antibiotics, penicillin-streptomycin. Varying concentrations of PROK2 and PROK1 were added to culture dish approximately thirty minutes after plating. Proliferation was measured visually and individual rings were photographed to record results. Both PROK2 and PROK1 induced a proliferative response at concentrations ranging from 1 to 100 ng/ml. This mitogenic effect was observed in aortas from the animals at all three ages. PROK2 was also tested in the rat corneal model of anigiogenesis where no effect was noted. The observed angiogenic effect in the aortic ring cultures may be due to the mitogenic effects of the GROα homologue.

### Example 6

### Baculovirus Expression of PROK2

An expression vector containing a GLU-GLU tag, pzBV32L:PROK2cee, was designed and prepared to express PROK2cee polypeptides in insect cells.

### A. Expression Vector:

An expression vector, pzBV32L:PROK2cee, was prepared to express human PROK2 polypeptides having a carboxy-terminal Glu-Glu tag, in insect cells as follows.

A 371 bp fragment containing sequence for PROK2 and a polynucleotide sequence encoding EcoR1 and Xba1 restriction sites on the 5′ and 3′ ends, respectively, was generated by PCR amplification using PCR SuperMix (Gibco BRL, Life Technologies) and appropriate buffer from a plasmid containing PROK2 cDNA (PROK2-zyt-1.contig) using primers ZC29463 (SEQ ID NO:23) and ZC29462 (SEQ ID NO:24). (Note: the PROK2 sequence and the Xba1 site was out of frame. An additional 2 bases, CC—antisense, were added to put in frame, which coded for an additional Gly between the PROK2 sequence and the CEE tag.) The PCR reaction conditions were as follows: 1 cycle of 94° C. for 3 minutes, followed by 25 cycles of 94° C. for 30 seconds, 50° C. for 30 seconds, and 68° C. for 30 seconds; followed by a 4° C. hold. The fragment was visualized by gel electrophoresis (1% Agarose-1 μl of 10 mg/ml EtBr per 10 ml of agarose). A portion of the PCR product was digested with EcoR1 and Xba1 restriction enzymes in appropriate buffer, then run on an agarose gel. DNA corresponding to the EcoR1/Xba1 digested PROK2 coding sequence was excised, purified using Qiagen Gel Extraction kit (#28704), and ligated into an EcoR1/XbaI digested baculovirus expression donor vector, pZBV32L. The pZBV32L vector is a modification of the pFastBac1™ (Life Technologies) expression vector, where the polyhedron promoter has been removed and replaced with the late activating Basic Protein Promoter. In addition, the coding sequence for the Glu-Glu tag (SEQ ID NO:10) as well as a stop signal is inserted at the 3′ end of the multiple cloning region. About 216 nanograms of the restriction digested PROK2 insert and about 300 ng of the corresponding vector were ligated overnight at 15° C. One μl of ligation mix was electroporated into 35 μl DH10B cells (Life Technologies) at 2.1 kV. The electroporated DNA and cells were diluted in 1 ml of LB media, grown for 1 hr at 37° C., and plated onto LB plates containing 100 μg/ml ampicillin. Clones were analyzed by restriction digests and one positive clone was selected and streaked on AMP+ plates to get single colonies for confirmation by sequencing.

Sequencing revealed the presence of a initiation codon upstream of the actual start codon which would possibly interfere with proper translation. Therefore, the upstream codon was removed using a Quick-change mutagenesis kit from Stratagene (La Jolla, Calif.). This was accomplished by designing forward and reverse primers that changed the upstream initiation ATG to a ATC, thereby also eliminating a Nco restriction digest site and creating a Sma1 site instead. The new mutagenized plasmid containing the Sma1 and Xba1 cleavage sites at the 5′ and 3′ ends of the PROK2 sequence was then electroporated into DH10B cells as before, analyzed by restriction digests, this time with Sma1 and Xba1, and a positive clone was selected and streaked on AMP+ plates to get a single colony for confirmation by sequencing as before. A clone for the PROK2 polynucleotide sequence could also be cloned without the upstream initiation codon.

One to 5 ng of the positive clone donor vector was transformed into 100 μl DH10Bac Max Efficiency competent cells (GIBCO-BRL, Gaithersburg, Md.) according to manufacturer's instruction, by heat shock for 45 seconds in a 42° C. waterbath. The transformed cells were then diluted in 980 μl SOC media (2% Bacto Tryptone, 0.5% Bacto Yeast Extract, 10 ml 1M NaCl, 1.5 mM KCl, 10 mM MgCl₂, 10 mM MgSO₄ and 20 mM glucose) out-grown in shaking incubator at 37° C. for four hours and plated onto Luria Agar plates containing 50 μg/ml kanamycin, 7 μg/ml gentamicin, 10 μg/ml tetracycline, IPTG and Blue Gal. The plated cells were incubated for 48 hours at 37° C. A color selection was used to identify those cells having PROK2cee encoding donor insert that had incorporated into the plasmid (referred to as a “bacmid”). Those colonies, which were white in color, were picked for analysis. Bacmid DNA was isolated from positive colonies using standard isolation technique according to Life Technologies directions. Clones were screened for the correct insert by amplifying DNA using primers to the transposable element in the bacmid via PCR. The PCR reaction conditions were as follows: 35 cycles of 94° C. for 45 seconds, 50° C. for 45 seconds, and 72° C. for 5 minutes; 1 cycle at 72° C. for 10 min.; followed by 4° C. soak. The PCR product was run on a 1% agarose gel to check the insert size. Those having the correct insert size were used to transfect Spodoptera frugiperda (Sf9) cells. The polynucleotide sequence is shown in SEQ ID NO:25. The corresponding amino acid sequence is shown inis shown in SEQ ID NO:26.

### B. Transfection in Insect Cells:

Sf9 cells were seeded at 1×10⁶ cells per 35 mm plate and allowed to attach for 1 hour at 27° C. Five micrograms of bacmid DNA was diluted with 100 μl Sf-900 II SFM medium (Life Technologies, Rockville, Md.). Fifteen μl of lipofectamine Reagent (Life Technologies) was diluted with 100 μl Sf-900 II SFM. The bacmid DNA and lipid solutions were gently mixed and incubated 30-45 minutes at room temperature. The media from one plate of cells was aspirated. Eight hundred microliters of Sf-900 II SFM was added to the lipid-DNA mixture. The DNA-lipid mix was added to the cells. The cells were incubated at 27° C. overnight. The DNA-lipid mix was aspirated the following morning and 2 ml of Sf-900 II media was added to each plate. The plates were incubated at 27° C., 90% humidity, for 168 hours after which the virus was harvested.

### C. Primary Amplification

Sf9 cells were seeded at 1×10⁶ cells per 35 mm plate and allowed to attach for 1 hour at 27° C. They were then infected with 500 μl of the viral stock from above and incubated at 27° C. for 4 days after which time the virus was harvested according to standard methods known in the art.

### D. Secondary Amplification

Sf9 cells were seeded at 1×10⁶ cells per 35 mm plate and allowed to attach for 1 hour at 27° C. They were then infected with 20 μl of the viral stock from above and incubated at 27° C. for 4 days after which time the virus was harvested according to standard methods known in the art.

### E. Tertiary Amplification

Sf9 cells were grown in 80 ml Sf-900 II SFM in 250 ml shake flask to an approximate density of 1×10⁶ cells/ml. They were then infected with 200 μl of the viral stock from above and incubated at 27° C. for 4 days after which time the virus was harvested according to standard methods known in the art.

### F. Expression of PROK2cee

Third round viral stock was titered by a growth inhibition curve and the culture showing an MOI of “1” was allowed to proceed for 48 hrs. The supernatant was analyzed via Western blot using a primary monoclonal antibody specific for the n-terminal Glu Glu epitope and a HRP conjugated Gt anti Mu secondary antibody. Results indicated a band of the predicted molecular weight.

A large viral stock was then generated by the following method: Sf9 cells were grown in 1 L Sf-900 II SFM in a 2800 ml shake flask to an approximate density of 1×10⁶ cells/ml. They were then infected with viral stock from the 3^{rd} round amp. and incubated at 27° C. for 72 hrs after which time the virus was harvested. Larger scale infections were completed to provide material for downstream purification.

### Example 7

### Expression in E. coli

### A. Generation of the Native PROK2 Expression Construct

A DNA fragment of native PROK2 (SEQ ID NO:11) was isolated using PCR. Primer zc #40,821 (SEQ ID NO:12) containing 41 bp of vector flanking sequence and 24 bp corresponding to the amino terminus of PROK2, and primer zc#40,813 (SEQ ID NO:13) contained 38 bp corresponding to the 3′ end of the vector which contained the PROK2 insert. Template was pZBV32L:PROK2cee. The PCR conditions were as follows: 25 cycles of 94° C. for 30 seconds, 50° C. for 30 seconds, and 72° C. for 1 minute; followed by a 4° C. soak. A small sample (2-4 μL) of the PCR sample was run on a 1% agarose gel with 1×TBE buffer for analysis, and the expected band of approximately 500 bp fragment was seen. The remaining volume of the 100 μL reaction was precipitated with 200 μL absolute ethanol. Pellet was resuspended in 10 μL water to be used for recombining into Sma1 cut recipient vector pTAP238 to produce the construct encoding the PROK2 as disclosed above. The clone with correct sequence was designated as pTAP432. It was digested with Not1/Nco1 (10P DNA, 5 μl buffer 3 New England BioLabs, 2 μL Not 1, 2 μL Nco 1, 31 μL water for 1 hour at 37° C.) and religated with T4 DNA ligase buffer (7 μL of the previous digest, 2 μL of 5× buffer, 1 μL of T4 DNA ligase). This step removed the yeast sequence, CEN-ARS, to streamline the vector. The DNA was diagnostically digested with Pvu 2 and Pst 1 to confirm the absence of the yeast sequence. DNA was transformed into E. coli strain W3110/pRARE.

B. Expression of the Native PROK2 in E. coli

E. coli was inoculated into 100 ml Superbroth II medium (Becton Dickinson, Franklin Lakes, N.J.) with 0.01% Antifoam 289 (Sigma), 30 μg/ml kanamycin, 35 μg/ml chloramphenicol and cultured overnight at 37° C. A 5 ml inoculum was added to 500 ml of the same medium in a 2 L culture flask which was shaken at 250 rpm at 37° C. until the culture attained an OD₆₀₀ of 4. IPTG was then added to a final concentration of 1 mM and shaking was continued for another 2.5 hours. The cells were centrifuged at 4,000×g for 10 min at 4° C. The cell pellets were frozen at −80° C.

### Example 8

### Codon Optimization

### A. Generation of the Codon Optimized PROK2 Expression Construct

Native human PROK2 gene sequence could not be expressed in E. coli strain W3110. Examination of the codons used in the PROK2 coding sequence indicated that it contained an excess of the least frequently used codons in E. coli with a CAI value equal to 0.211. The CAI is a statistical measure of synonymous codon bias and can be used to predict the level of protein production (Sharp et al., Nucleic Acids Res. 15(3):1281-95, 1987). Genes coding for highly expressed proteins tend to have high CAI values (>0.6), while proteins encoded by genes with low CAI values (≦0.2) are generally inefficiently expressed. This suggested a reason for the poor production of PROK2 in E. coli. Additionally, the rare codons are clustered in the second half of the message leading to higher probability of translational stalling, premature termination of translation, and amino acid misincorporation (Kane J F. Curr. Opin. Biotechnol. 6(5):494-500, 1995).

It has been shown that the expression level of proteins whose genes contain rare codons can be dramatically improved when the level of certain rare tRNAs is increased within the host (Zdanovsky et al., ibid., 2000; Calderone et al., ibid., 1996; Kleber-Janke et al., ibid., 2000; You et al., ibid., 1999). The pRARE plasmid carries genes encoding the tRNAs for several codons that are rarely used E. coli (argU, argW, leuW, proL, ileX and glyT). The genes are under the control of their native promoters. Co-expression with pRARE enhanced PROK2 production in E. coli and yielded approximately 100 mg/L. Co-expression with pRARE also decreased the level of truncated PROK2 in E. coli lysate. These data suggest that re-resynthesizing the gene coding for PROK2 with more appropriate codon usage provides an improved vector for expression of large amounts of PROK2.

The codon optimized PROK2 coding sequence (SEQ ID NO:14) was constructed from six overlapping oligonucleotides: zc45,048 (SEQ ID NO:15), zc45,049 (SEQ ID NO:16), zc45,050 (SEQ ID NO:17), zc45,051 (SEQ ID NO:18), zc45,052 (SEQ ID NO:19) and zc45,053 (SEQ ID NO:20). Primer extension of these overlapping oligonucleotides followed by PCR amplification produced a full length PROK2 gene with codons optimized for expression in E. coli. The final PCR product was inserted into expression vector pTAP237 by yeast homologous recombination. The expression construct was extracted from yeast and transformed into competent E. coli DH10B. Clones resistance to kanamycin were identified by colony PCR. A positive clone was verified by sequencing and subsequently transformed into production host strain W3110. The expression vector with the optimized PROK2 sequence was named pSDH187. The resulting gene was expressed very well in E. coli. Expression levels with the new construct increased to around 150 mg/L.

B. Expression of the Codon Optimized PROK2 in E. coli

E. coli was inoculated into 100 ml Superbroth II medium (Becton Dickinson) with 0.01% Antifoam 289 (Sigma), 30 μg/ml kanamycin and cultured overnight at 37° C. A 5 ml inoculum was added to 500 ml of same medium in a 2 L culture flask which was shaken at 250 rpm at 37° C. until the culture attained an OD₆₀₀ of 4. IPTG was then added to a final concentration of 1 mM and shaking was continued for another 2.5 hours. The cells were centrifuged at 4,000×g for 10 min at 4° C. The cell pellets were frozen at −80° C. until use at a later time.

### Example 9

### Purification and Refolding of PROK2 Produced in E. coli

### A. Inclusion Body Isolation:

Following induction of protein expression in either batch ferment or shaker flask culture, the E. coli broth was centrifuged in 1 liter bottles at 3000 RPM in a Sorvall swinging bucket rotor. Additional washing of the cell paste to remove any broth contaminants was performed with 50 mM Tris pH 8.0 containing 200 mM NaCl and 5 mM EDTA until the supernate was clear.

The cell pellets were then suspended in ice cold lysis buffer (50 mM Tris pH 8.0; 5 mM EDTA; 200 mM NaCl, 10% sucrose (w/v); 5 mM DTT; 5 mM Benzamidine;) to 10-20 Optical Density units at 600 nm. This slurry was then subjected to 2-3 passes at 8500-9000 psi in a chilled APV 2000 Lab Homogenizer producing a disrupted cell lysate. The insoluble fraction (inclusion bodies) was recovered by centrifugation of the cell lysate at 20,000×G for 1 hour at 4° C.

The inclusion body pellet (resulting from the 20,000×G spin) was re-suspended in wash buffer (50 mM Tris pH 8 containing 200 mM NaCl, 5 mM EDTA, 5 mM DTT, 5 mM Benzamidine) at 10 ml wash buffer per gram inclusion bodies, and was completely dispersed utilizing an OMNI international rotor stator generator. This suspension was centrifuged at 20,000×G for 30 minutes at 4° C. The wash cycle was repeated 3-5 times until the supernatant was clear.

The final washed pellet was solubilized in 8M Urea, 50 mM Borate buffer at pH 8.6 containing 0.1M Sodium Sulfite and 0.05 M Sodium Tetrathionate at pH 8.2. The solubilization and sulfitolysis reaction was allowed to proceed at 4° C. overnight with gentle shaking. The resulting pinkish colored solution was centrifuged at 35,000×g for 1 hour at 4° C. and the clarified supernate, containing the soluble PROK2, was 0.45 um filtered.

### B. PROK2 Refolding:

The solubilized PROK2 was refolded by drop-wise dilution into ice cold refolding buffer containing 55 mM Borate pH 8.6, 1.0 M Arginine, 0.55 M Guanidine HCL, 10.56 mM NaCl, 0.44 mM KCl, 0.055% PEG, 10 mM reduced Glutathione and 1.0 mM oxidized Glutathione at a final PROK2 concentration of 100-150 ug/ml. Once diluted, the mixture was allowed to stir slowly in the cold room for 48-72 hours.

### C. Product Recovery & Purification:

After refolding, the solution was clarified by centrifugation at 22,000×G, 1 hour, 4° C. and/or by filtration using a 0.45 micron membrane. The clarified supernate, containing refolded PROK2, was adjusted to 50 mM acetate and the pH adjusted to 4.5 with addition of HCl. The pH adjusted material was captured by cation exchange chromatography on a Pharmacia Streamline SP column (33 mm ID×65 mm length) equilibrated in 50 mM acetate pH 4.5 buffer. The load flow rate was 10 ml/min with inline dilution proportioning 1:5 in 50 mM acetate buffer at pH 4.5. This dilution lowers the ionic strength enabling efficient binding of the target to this matrix. After sample loading was complete, the column was washed to baseline absorbance with equilibration buffer prior to step elution with 50 mM acetate pH 4.5 buffer containing 1 M NaCl.

The eluate pool from the cation exchange step was brought to 1% Acetic acid, pH 3.0 and Loaded to a column (22 mm×130 mm) containing Toso Hass Amberchrom CG71m reverse phase media equilibrated in 1% acetic acid, pH 3.0 at a flow rate of 10 ml/min. Upon washing to baseline absorbance, the column was eluted with a 20 column volume gradient formed between equilibration buffer and 99% (V/V) acetonitrile, 1% (V/V) acetic acid.

The eluate pool from the reverse phase step was subjected to another round of cation exchange chromatography. The pool was directly loaded on to a Toso Haas SP 650 S column (10 mm×50 mm) equilibrated in 50 mM acetate pH 4.5 buffer at a flow rate of 3 ml/min. Upon completing the sample load, and washing to baseline absorbance, the column was step eluted with 50 mM acetate pH 3.0 buffer containing 1.0 M NaCl. The protein eluate pool was concentrated against a 3 k Da cutoff ultrafiltration membrane using an Amicon concentration unit in preparation for the final purification and buffer exchange size exclusion step.

### D. Size Exclusion Buffer Exchange and Formulation:

The concentrated cation pool was injected onto a Pharmacia Superdex Peptide size exclusion column (Pharmacia, now Pfizer, La Jolla, Calif.) equilibrated in 25 mM Histidine; 120 mM NaCl at pH 6.5. The symetric eluate peak containing the product was pooled, 0.2 micron sterile-filtered, aliquoted and stored at −80° C.

### Example 10

### Activity of PROK2 and PROK1 in a Reporter Assay

### A. Cell Lines

Rat2 fibroblast cells (ATCC #CRL-1764, American Type Culture Collection, Manassass, Va.) were transfected with a SRE luciferase reporter construct and selected for stable clones. These were then transfected with constructs for either GPCR73a receptor (SEQ ID NO:21) or GPCR73b receptor (SEQ ID NO:22).

### B. Assay Procedure

Cells were trypsinized and seeded in Corning 96-well white plates at 3,000 cells/well in media containing 1% serum and incubated overnight at 37° C. and 5% CO₂. Media was removed and samples were added in triplicate to cells in media containing 0.5% BSA and incubated for four hours at 37° C. and 5% C0₂. After media was removed the cells were lysed and luciferase substrate was added according to the Promega luciferase assay system (Promega Corp., Madison, Wis.)

### C. Data and Conclusions

All data were reported as fold-induction of the RLU (relative light units) from the luminometer divided by the basal signal (media only). PROK2 was prepared in house. PROK1 used in the assay was purchased from PeproTech Inc. (Rocky Hill, N.J.).

Tables 3 and 4 show that PROK2 was more active than PROK1 in a dose-dependent manner with cells expressing the GPCR73a receptor.

TABLE 3
GPCR 73a Fold-induction

| conc. (ng/ml) | PROK2 (E. coli produced) | PROK1 |
|---|---|---|
| 1000 | 17.8 | 20 |
| 320 | 20.7 | 24.4 |
| 100 | 19 | 11.4 |
| 32 | 15 | 5.8 |
| 10 | 8.4 | 2.5 |
| 3.2 | 4 | 1.6 |
| 1 | 1.9 | 1.2 |

TABLE 4
GPCR73a Fold-induction

| conc. (ng/ml) | PROK2 (E. coli produced) | PROK1 |
|---|---|---|
| 1000 | 13.9 | 15 |
| 320 | 22 | 20.5 |
| 100 | 17.6 | 11.4 |
| 32 | 14.1 | 7.2 |
| 10 | 10.2 | 2.6 |
| 3.2 | 7.6 | 1.3 |
| 1 | 4.1 | 0.95 |

Tables 5 and 6 show that PROK2 and PROK1 were similar in activity with the cells expressing the GPCR73b receptor. Activity of both molecules was lower in the cells expressing the GPCR73b receptor. It is not known if the GPCR73b receptor numbers were equivalent in both cell lines.

TABLE 5
GPCR73b Fold-induction

| conc. (ng/ml) | PROK2 (E. coli produced) | PROK1 |
|---|---|---|
| 1000 | 7.1 | 8.4 |
| 320 | 6.3 | 8.3 |
| 100 | 4.7 | 5.6 |
| 32 | 3 | 2.8 |
| 10 | 1.9 | 1.8 |
| 3.2 | 1.3 | 1.3 |
| 1 | 0.7 | 1.1 |

TABLE 6
GPCR73b Fold-induction

| conc. (ng/ml) | PROK2 (E. coli produced) | PROK1 |
|---|---|---|
| 1000 | 4.8 | 6.1 |
| 320 | 5.2 | 5.8 |
| 100 | 4.4 | 4.1 |
| 32 | 2.6 | 2.7 |
| 10 | 1.7 | 1.8 |
| 3.2 | 1.2 | 1.4 |
| 1 | 1 | 1.1 |

Table 7 shows that Baculovirus-expressed PROK2 that has been heated at 56° C. for 30 minutes may have reduced activity than fresh PROK2.

TABLE 7
GPCR73a Fold-induction

| conc. (ng/ml) | Fresh PROK2 | Heated PROK2 |
|---|---|---|
| 100 | 20.5 | 18.6 |
| 32 | 18.7 | 14.8 |
| 10 | 13.1 | 10 |
| 3.2 | 7.1 | 3.7 |
| 1 | 2.5 | 1.8 |

### Example 11

### MIP-2 Detection in Lavage Fluids and Serum of Mice Following IP (Intraperitoneal) Injection of PROK2

As discussed in Example 3, above, mouse KC is the mouse homolog of human GROα, and CINC-1 is the rat homolog. Similarly, increased MIP-2 expression has been found to be associated with neutrophil influx in various inflammatory conditions. See Banks, C. et al, J. Path. 199: 28-35, 2003.

Similar to the methods used in Example 3, four groups of ten mice were injected with PROK2 at 5 and 50 ug/kg, a vehicle control, or no treatment. These mice weighed approximately 20 grams, so the dose was 5 μg/kg. MIP-2 levels were measured in both peritoneal lavage fluid and serum using a Quantikine M Murine mouse MIP-2 ELISA kit (R and D Systems, Minneapolis, Minn.). Test results are shown in Table 8.

TABLE 8
MIP-2 picograms/ml

| | Serum | Lavage Fluid |
|---|---|---|
| Non-treated control | 6.2 +/− 1.3 | 5.9 +/− 0.7 |
| Vehicle | 6.7 +/− 1.3 | 16.7 +/− 2.2 |
| 5 ug/kg PROK2 | 14.3 +/− 2.7 | 21.5 +/− 3.7 |
| 50 ug/kg PROK2 | 7.7 +/− 1.8 | 8.7 +/− 1.2 |

Data = mean +/− SEM

Conclusions: MIP-2 is up-regulated in serum and lavage fluid in response to a low, (5 ug/kg), IP injection of PROK2. Concentrations in serum are approximately 2-fold higher in the PROK2 treated animals. There is a lesser effect in lavage fluid, but that is due to the fact that some activation took place in the vehicle treated animals over non-treated control animals. At the higher (50 ug/kg dose) no effect was observed suggesting that at elevated doses there is no chemotactic effect. These results correlate with the neutrophil numbers, where in, neutrophil infiltration was observed only in the animals administered the lower (5 ug/kg) dose of PROK2.

### Example 12

### Production of PROK2 Polyclonal Antibodies

Polyclonal antibodies were prepared by immunizing 2 female New Zealand white rabbits with the purified recombinant protein huPROK2-CEE-Bv (SEQ ID NO:24) The rabbits were each given an initial intraperitoneal (ip) injection of 200 μg of purified protein in Complete Freund's Adjuvant followed by booster ip injections of 100 μg peptide in Incomplete Freund's Adjuvant every three weeks. Seven to ten days after the administration of the second booster injection (3 total injections), the animals were bled and the serum was collected. The animals were then boosted and bled every three weeks.

Polyclonal antibodies were purified from the immunized rabbit serum using a 5 ml Protein A sepharose column (Pharmacia LKB). Following purification, the polyclonal antibodies were dialyzed with 4 changes of 20 times the antibody volume of PBS over a time period of at least 8 hours. HuPROK2-specific antibodies were characterized by ELISA using 500 ng/ml of the purified recombinant protein huPROK2-CEE-Bv (SEQ ID NO:24) as the antibody target. The lower limit of detection (LLD) of the rabbit anti-huPROK2 purified antibody was 1 ng/ml on its specific purified recombinant antigen huPROK2-CEE-Bv.

### Example 13

### Detection of PROK2 Protein

The purified polyclonal huPROK2 antibodies were characterized for their ability to bind recombinant human PROK2 polypeptides using the ORIGEN® Immunoassay System (IGEN Inc, Gaithersburg, Md.). In this assay, the antibodies were used to quantitatively determine the level of recombinant huPROK2 in rat serum samples. An immunoassay format was designed that consisted of a biotinylated capture antibody and a detector antibody, which was labeled with ruthenium (II) tris-bipyridal chelate, thereby sandwiching the antigen in solution and forming an immunocomplex. Streptavidin-coated paramagnetic beads were then bound to the immunocomplex. In the presence of tripropylamine, the ruthenylated Ab gave off light, which was measured by the ORIGEN analyzer. Concentration curves of 0.1-50 ng/ml huPROK2 made quantitation possible using 50 microliters of sample. The resulting assay exhibited a lower limit of detection of 200 pg/ml huPROK2 in 5% normal rat serum.

### Example 14

### PROK2 and Inflammatory Bowel Disease (IBD)

The purpose was to determine if PROK2 expression was up-regulated in IBD, intestinal tissue biopsies from six ulcerative colitis (UC) patients, seven Crohn's disease patients, and four normal donor controls were analyzed using Taqman RTPCR. Tissue biopsies were obtained from two sites in the intestine from each individual donor, one site with no or low amounts of inflammation and one diseased site. In some instances, no unaffected areas could be found. Sites of biopsy obtainment included: Cecum, rectum, transverse, ascending, and descending colon, terminal ileum, and signum.

Immediately following biopsy, tissues were flash frozen in liquid nitrogen. Tissue was crushed and resuspended in lysis buffer: 2% SDS, 20 mM Tris (pH 7.4), and 2% Phosophotase Inhibitor Cocktail (Sigma, Saint Louis, Mo.). RNA was prepared using RNeasy kits from (Qiagen, Valencia, Calif.), following manufacturer's instructions. Taqman EZ RT-PCR Core Reagent Kit (Applied Biosystems, Foster City, Calif.) was used to determine PROK2 expression levels.

Following manufacturer's instructions a PROK2 standard curve was prepared using human testis RNA at different concentrations (250 ng/μl, 50 ng/μl, 12.5 ng/μl and 3.125 ng/μl). These standard curve dilutions were first used to test the primers designed for PROK2 gene and for a housekeeping gene (human glucuronidase (GUS). Once the working conditions of primer and standard curve were established, intestinal disease RNA samples were tested. The RNA samples were thawed on ice and then were diluted to 50 ng/μl in RNase-free water (Invitrogen, Cat #750023). Diluted samples were kept on ice all the time.

Using the TaqMan EZ RT-PCR Core Reagent Kit (Applied Biosystems, Cat# N808-0236), master mix was prepared for both PROK2 and for a housekeeping gene (GUS). To assay samples in triplicate, 3.5 μl of each RNA samples were aliquoted. For positive controls, 3.5 μl each standard curve dilutions were used in place of sample RNA. For the negative control, 3.5 μl RNase-free water was used for a no template control. For endogenous controls (human GUS message), 3.5 μl of both standard curve dilutions and the sample RNAs were aliquoted. Then 84 μl of PCR master mix was added and mixed well by pipetting. A MicroAmp Optical 96-well Reaction Plate (Applied Biosystems Cat# N801-0560) was placed on ice and 25 μl of RNA/master mix was added in triplicates to the appropriate wells. Then MicroAmp 12-Cap Strips (Applied Biosystems Cat# N801-0534) were used to cover entire plate. The plate was then spun for two minutes at 3000 RPM in the Qiagen Sigma 4-15 centrifuge.

The samples were run on a PE-ABI 7700 (Perkin Elmer, now EG&G, Inc. Wellesley, Mass.). Sequence Detector was launched and the default was set to Real Time PCR. Fluorochrome was set to FAM. Plate template was set to indicate where standards and where unknown test samples were.

Expression for each sample was reported as a Ct value. The Ct value was the point at which the fluorochrome level or RT-PCR product (a direct reflection of RNA abundance) was amplified to a level, which exceeds the threshold or background level. The lower the Ct value, the higher the expression level, since RT-PCR of a highly expressing sample results in a greater accumulation of fluorochrome/product which crosses the threshold sooner. A Ct value of 40 indicates that there was no product measured and should result in a mean expression value of zero. The Ct was converted to relative expression value based on comparison to the standard curve. For each sample was being tested, the amount of PROK2 and GUS expression level was determined from the appropriate standard curve. Then these calculated PROK2 expression values were divided by the GUS expression value for each sample in order to obtain a normalized PROK2 expression value for each sample.

Results: In the four normal donor tissues, PROK2 relative expression was extremely low (mean 0.07+/−0.07 SEM). In both UC and Crohn's diseased tissues, PROK2 expression was significantly elevated compared to the expression seen in normal donors. Mean relative PROK2 expression in UC and Crohn's patients with minimally inflamed tissue was: 4.9+/−10 SEM in UC, and 1.45+/−0.8 SEM in Crohn's.

Mean fold-increase over normal donors was 70-fold in UC and 20.7-fold in Crohn's. In the inflamed tissue samples, PROK2 expression was even higher. Mean fold PROK 1 expression in inflamed UC tissue was 15.8+/−18.5 SEM and 40.8+/−92.8 SEM in Crohn's disease inflamed tissue. Mean fold increase in PROK2 expression over normals in UC was 213-fold and in Crohn's was 583-fold.

All thirteen UC and Crohn's donor inflamed intestinal tissue biopsies had PROK2 expression levels higher than the mean normal donor biopsies.

Conclusions: PROK2 has been shown to induce chemokine release both in vitro and in vivo. See Examples 2 and 3 above. Furthermore, following IP injection in mice, two potent chemokines, mouse KC (as shown in Example 3) and MIP-2 (as shown in Example 11) can be measured in the peritoneum and the blood stream, accompanied by an influx of neutrophils. Additionally, as shown in this Example, PROK2 was up-regulated in intestinal tissues obtained from inflammatory bowel disease patients suggesting that it may be involved in the inflammatory process and the progression of IBD.

These results are consistent with studies that show that chemokines are chemotactic cytokines that are able to promote leukocyte migration to areas of inflammation and have recently been implicated in the pathophysiology of many disease states, including IBD. Mucosal changes in IBD were characterized by ulcerative lesions accompanied by prominent cellular infiltrates in the bowel.

### Example 15

### Measurements of PROK2 in Irritable Bowel Syndrome

In order to determine if PROK2 expression is dys-regulated in IBS, circulating levels were measured in plasma samples from women approximately 20-45 years of age that were carefully screened for the presence of current IBS symptoms. Samples were obtained from donors displaying mild or moderate IBS symptoms. An equal number of healthy control donor plasmas were also obtained. The non-symptomatic group denied any history of IBS or IBS-like GI symptoms or poor sleep. In addition, all studies were performed within the same menstrual cycle phase to control for potential cycle phase differences. A total of twelve plasma samples were obtained during the night for the measurement of stress related hormones and PROK2 (prokineticin 2). Blood was drawn at 8:00 p.m. (20 hours), and hourly there after until 7:00 a.m. (7 hours).

### A. Platelet-Rich Plasma Preparation:

Approximately 4.5 ml of blood was collected into EDTA tubes and mixed by gentle inversion. Samples were stored on ice until all samples have been collected. Blood was centrifuged for 10 minutes at 200×g at 4° with brake off. The plasma fraction was decanted and aliquoted into tubes and frozen at −80° C.

Samples were stored frozen until the day they were assayed for PROK2 levels. Upon thawing, samples were spun at 13,000 rpm for 5 minutes at room temperature to remove any debris. Plasmas were diluted 1:4 in ELISA-B buffer (1% BSA in ELISA-C buffer) and each individual sample was run in triplicate.

### B. ELISA:

A sandwich based ELISA protocol was used to assay the plasma samples for circulating PROK2. Nunc-Immuno 96-well Maxisorp Surface ELISA plates were coated with a polyclonal rabbit anti-human antibody at a concentration of 1.06 μg/ml, which was prepared in ELISA-A buffer (0.1 M Na₂CO₃, pH 9.6). Then plates were sealed and incubated overnight at 4° C.

The next day, the plates were washed 5 times with ELISA-C buffer (1×PBS, 0.05% v/v Tween 20) and then they were blocked twice with SuperBlock (Pierce, Cat #37515) at room temperature for 5 minutes. Plates were washed 5 times with ELISA-C buffer before adding the samples and the standards to the plate.

For standard curve preparation, pooled platelet-rich plasma was prepared. Briefly, blood from four healthy individuals was drawn into EDTA containing tubes. Blood was spun at 200×g at 4° C. for 10 minute. Plasma from all four donors was pooled and aliquots were kept at −80° C.

On assay day, frozen platelet-rich plasma was thawed and spun for 5 minutes at 10,000 rpm to remove debris. Both standard curve plasma and human patient test plasmas were diluted 1:4 in ELISA-B buffer. E. coli produced PROK2 protein was spiked into the standard curve plasma at known concentrations to prepare a standard curve. Dilution series ran from 25 ng/ml to 0.08 ng/ml.

Both standard curve dilutions and samples were added to the plates in triplicate. Plates were sealed and incubated at 37° C. for 2 hours on a shaker. After the incubation, plates were washed five times with ELISA-C buffer.

For detection, biotinylated rabbit anti-human polyclonal PROK-1 antibody was diluted to 500 ng/ml in ELISA-B buffer. The ELISA plates were coated with antibody and incubated at 37° C. for an hour on a shaker. Following the incubation, plates were washed with ELISA-C buffer. Strepavidin horse radish peroxidase SA-HRP (Pierce) was diluted to 250 ng/ml in ELISA-B buffer and added to the plates. Plates were sealed and incubated at 37° C. for an hour on a shaker. After this incubation period, the plates were washed with ELISA-C buffer and Tetra methyl benzidine (TMB) solution (BioFX, Cat# TMBW-10000-01) was added to the plates at room temperature and incubated for 30 minutes on the bench. Color development was stopped with Stop Solution (BioFX 450 Stop Reagent, Cat# STPR-1000-01) and the absorbance at 450 nm minus 540 nm was read on a spectrophotometer (Molecular Devices) within 15 minutes of stop. Protein amounts were calculated from the standard curve using the SoftMax Pro software program.

### C. Results:

Control donor samples show lower levels of PROK2. While levels of PROK2 were highest in the samples drawn prior to midnight and after and including the 6:00 a.m, no PROK2 expression was detecting in control donors between midnight and 6:00 a.m. The final concentration of PROK2/ml was relatively low, with maximal values reaching levels of approximately 119 picograms/ml.

In the IBS donors, both the amounts of circulating PROK2 were higher than controls, and the pattern of expression was different, with expression observed throughout the night. Maximal PROK2 levels were approximately 9-fold higher, at 917 picograms/ml in the IBS patients. In addition, unlike the control donors, circulating PROK2 was detected in the samples obtained throughout the night (from midnight until 7:00 a.m).

### D. Conclusions:

In normal control patients, PROK2 expression follows a circadian pattern, with levels at there highest in the night and in the morning when the digestive process is either active, or commencing. In the IBS patients, this circadian pattern of expression is dys-regulated, suggesting PROK2 is involved in the pathology of IBS and contributes to the IBS syndrome. PROK2's profound effect on gut motility, both in the organ bath and in vivo, also support a connection to the altered intestinal motility symptoms related to IBS. A PROK2 antagonist could relieve the symptoms of constipation (or diarrhea), sleeplessness, abdominal bloating and increased sensitivity to pain sensation experienced in IBS patients.

### Example 16

### Expression of GPR73a and GPR73b in Rat Gastrointestinal Tract

Rats were fasted overnight and sacrificed. Intestines and stomachs were isolated and four-centimeter tissue sections from the stomach through the end of the colon were immediately flash frozen in liquid nitrogen. Acid-Phenol extraction method was used for RNA isolation. Briefly, tissue sections were grinded in liquid nitrogen then lysed/homogenized in acid guanidium based lysis buffer (4M Guanidine isothyocyanate, 25 mM sodium citrate (pH 7), 0.5% sarcosyl), NaOAc (0.1M final concentration) +βME (1:100). Lysates were spun down; supernatants were mixed with equal volume of acid phenol and 1/10 volume chloroform. After spinning down, equal volume of Isopropanol was added to the aqueous layer. Samples were incubated at −20° C. then pelleted down by spinning. Pellets were washed with 70% EtOH and then resuspended in DEPC treated water.

Taqman EZ RT-PCR Core Reagent Kit (Applied biosystems, Foster City, Calif.) was used to determine GPR73a and GPR73b receptor expression levels. Following manufacturer's instructions, a standard curve was prepared using one of the RNA isolates which had a high quality RNA and which showed expression of both receptors at the same level. Standard curve dilutions of this RNA sample were prepared at the following concentrations: 500 ng/μl, 250 ng/μl, 100 ng/μl and 12.5 ng/μl. These standard curve dilutions were first used to test the primers designed for GPR73a and GPR73b genes and for a housekeeping gene, rodent glyceraldehyde-3-phosphate dehydrogenase (GAPDH). Once the working conditions of primer and standard curve were established, RNA samples isolated from rat were tested.

The RNA samples were thawed on ice and diluted to 100 ng/μl in RNase-free water (Invitrogen, Cat #750023). Diluted samples were kept on ice during the experiment. Using the TaqMan EZ RT-PCR Core Reagent Kit (Applied Biosystems, Cat# N808-0236), master mix was prepared for GPR73a, GPR73b receptors and for the house keeping gene. To assay samples in triplicate, 3.5 μl of each RNA samples were aliquoted. For positive controls, 3.5 μl of each standard curve dilutions were used in place of sample RNA. For the negative control, 3.5 μl RNase-free water was used for the no template control. For endogenous controls (rodent GAPDH message), 3.5 μl of both standard curve dilutions and the sample RNAs were aliquoted. Then 84 μl of PCR master mix was added and mixed well by pipetting.

A MicroAmp Optical 96-well Reaction Plate (Applied Biosystems Cat# N801-0560) was placed on ice and 25 μl of RNA/master mix was added in triplicates to the appropriate wells. Then MicroAmp 12-Cap Strips (Applied Biosystems Cat# N801-0534) were used to cover entire plate. Then the plate was spun for two minutes at 3000 RPM in the Qiagen Sigma 4-15 centrifuge.

The samples were run on a PE-ABI 7700 (Perkin Elmer, now EG&G, Inc. Wellesley, Mass.). Sequence Detector was launched and the default was set to Real Time PCR. Fluorochrome was set to FAM. Plate template was set to indicate where standards and where unknown test samples were.

Expression for each sample is reported as a Ct value. The Ct value is the point at which the fluorochrome level or RT-PCR product (a direct reflection of RNA abundance) is amplified to a level, which exceeds the threshold or background level. The lower the Ct value, the higher the expression level, since RT-PCR of a highly expressing sample results in a greater accumulation of fluorochrome/product which crosses the threshold sooner. A Ct value of 40 means that there was no product measured and should result in a mean expression value of zero. The Ct is converted to relative expression value based on comparison to the standard curve. For each sample tested, the amount of GPR73a, GPR73b and GAPDH expression level was determined from the appropriate standard curve. Then these calculated expression values of GPR73a and GPR73b were divided by the GAPDH expression value of each sample in order to obtain a normalized expression for each sample. Each normalized expression value was divided by the normalized-calibrator value to get the relative expression levels. Using GraphPad Prism software, these normalized values were converted to fractions in which the highest expression level was indicated as 1.

TABLE 9
Normalized values (represented in fractions) for GPR73a and GPR73b expressions in rat.

| | GPR73a | | | | GPR73b | | |
|---|---|---|---|---|---|---|---|
| | normalized | | | | normalized | | |
| Samples | value | StDev | N | Samples | value | StDev | N |
| Forestomach | 0.067 | 0.057 | 3 | Forestomach | 0.063 | 0.013 | 3 |
| Fundus | 0.003 | 0.023 | 3 | Fundus | 0.106 | 0.033 | 3 |
| Antrum | 0.000 | 0.016 | 3 | Antrum | 0.000 | 0.004 | 3 |
| Pylorus/Antrum | 0.041 | 0.016 | 3 | Pylorus/Antrum | 0.104 | 0.005 | 3 |
| Duodenum | 0.107 | 0.035 | 3 | Duodenum | 0.205 | 0.037 | 3 |
| Jejunum-1 | 0.102 | 0.035 | 3 | Jejunum-1 | 0.100 | 0.058 | 3 |
| 2 | 0.087 | 0.020 | 3 | 2 | 0.021 | 0.008 | 3 |
| 3 | 0.126 | 0.037 | 3 | 3 | 0.097 | 0.016 | 3 |
| 4 | 0.250 | 0.054 | 3 | 4 | 0.150 | 0.042 | 3 |
| 5 | 0.268 | 0.030 | 3 | 5 | 0.123 | 0.022 | 3 |
| 6 | 0.240 | 0.024 | 3 | 6 | 0.177 | 0.037 | 3 |
| 7 | 0.339 | 0.039 | 3 | 7 | 0.173 | 0.031 | 3 |
| 8 | 0.329 | 0.107 | 3 | 8 | 0.129 | 0.031 | 3 |
| 9 | 0.327 | 0.101 | 3 | 9 | 0.286 | 0.078 | 3 |
| 10 | 0.425 | 0.071 | 3 | 10 | 0.235 | 0.011 | 3 |
| 11 | 0.379 | 0.011 | 3 | 11 | 0.147 | 0.016 | 3 |
| 12 | 0.577 | 0.076 | 3 | 12 | 0.253 | 0.068 | 3 |
| 13 | 0.570 | 0.043 | 3 | 13 | 0.315 | 0.053 | 3 |
| 14 | 0.250 | 0.011 | 3 | 14 | 0.171 | 0.017 | 3 |
| 15 | 0.492 | 0.027 | 3 | 15 | 0.397 | 0.034 | 3 |
| 16 | 0.989 | 0.089 | 3 | 16 | 0.494 | 0.048 | 3 |
| 17 | 0.977 | 0.313 | 3 | 17 | 0.420 | 0.045 | 3 |
| 18 | 1.000 | 0.061 | 3 | 18 | 0.523 | 0.146 | 3 |
| Ileum-1 | 0.797 | 0.080 | 3 | Ileum-1 | 0.630 | 0.141 | 3 |
| 2 | 0.636 | 0.014 | 3 | 2 | 0.434 | 0.080 | 3 |
| 3 | 0.614 | 0.015 | 3 | 3 | 0.441 | 0.115 | 3 |
| 4 | 0.923 | 0.085 | 3 | 4 | 0.871 | 0.288 | 3 |
| 5 | 0.807 | 0.142 | 3 | 5 | 0.739 | 0.017 | 3 |
| 6 | 0.755 | 0.080 | 3 | 6 | 1.000 | 0.246 | 3 |
| Cecum | 0.088 | 0.020 | 3 | Cecum | 0.369 | 0.036 | 3 |
| Proximal | 0.171 | 0.060 | 3 | Proximal | 0.887 | 0.021 | 3 |
| Middle | 0.088 | 0.051 | 3 | Middle | 0.209 | 0.047 | 3 |
| Distal | 0.047 | 0.019 | 3 | Distal | 0.012 | 0.002 | 3 |

### Example 17

### PROK2 and Monoclonal Antibodies

Rat monoclonal antibodies are prepared by immunizing 4 female Sprague-Dawley Rats (Charles River Laboratories, Wilmington, Mass.), with the purified recombinant protein from Example 6 or Example 7, above. The rats are each given an initial intraperitoneal (IP) injection of 25 □g of the purified recombinant protein in Complete Freund's Adjuvant (Pierce, Rockford, Ill.) followed by booster IP injections of 10 □g of the purified recombinant protein in Incomplete Freund's Adjuvant every two weeks. Seven days after the administration of the second booster injection, the animals are bled and serum is collected.

The PROK2-specific rat sera samples are characterized by ELISA using 1 ug/ml of the purified recombinant protein PROK2 as the specific antibody target.

Splenocytes are harvested from a single high-titer rat and fused to SP2/0 (mouse) myeloma cells using PEG 1500 in a single fusion procedure (4:1 fusion ratio, splenocytes to myeloma cells, “Antibodies: A Laboratory Manual, E. Harlow and D. Lane, Cold Spring Harbor Press). Following 9 days growth post-fusion, specific antibody-producing hybridoma pools are identified by radioimmunoprecipitation (RIP) using the Iodine-125 labeled recombinant protein PROK2 as the specific antibody target and by ELISA using 500 ng/ml of the recombinant protein PROK2 as specific antibody target. Hybridoma pools positive in either assay protocol are analyzed further for their ability to block the cell-proliferative activity (“neutralization assay”) of purified recombinant protein PROK2 on Baf3 cells expressing the receptor sequence of GPR73a (SEQ ID NO:27) and/or GPR73b (SEQ ID NO:28).

Hybridoma pools yielding positive results by RIP only or RIP and the “neutralization assay” are cloned at least two times by limiting dilution.

Monoclonal antibodies purified from tissue culture media are characterized for their ability to block the cell-proliferative activity (“neutralization assay”) of purified recombinant PROK2 on Baf3 cells expressing the receptor sequences. “Neutralizing” monoclonal antibodies are identified in this manner.

A similar procedure is followed to identify monoclonal antibodies to PROK1 using the amino acid sequence in SEQ ID NO:5.

### Example 18

### Stimulation of Contractility in Guinea Pig Gastrointestinal Organ Bath Assay

Male Hartley Guinea pigs at six weeks of age weighing approximately 0.5 kg were euthanized by carbon monoxide. Intestinal tissue was harvested as follows: 2-3 cm longitudinal sections of ileum 10 cm rostral of the cecum, and 2-3 cm longitudinal sections of duodenum, jejunum, and proximal and distal colon.

Tissue was washed in Krebs Ringer's Bicarbonate buffer containing 118.2 mM NaCl, 4.6 mM KCl, 1.2 mm MgSO₄, 24.8 mM NaHCO₃, 1.2 mM KH₂P0₄, 2.5 mM CaCl₂ and 10 mM glucose. Following a thorough wash, the tissue was mounted longitudinally in a Radnoti organ bath perfusion system (SDR Clinical Technology, Sydney Australia) containing oxygenated Krebs buffer warmed and maintained at 37° C. A one gram pre-load was applied and the tissue strips were allowed to incubate for approximately 30 minutes. Baseline contractions were then obtained. Isometric contractions were measured with a force displacement transducer and recorded on a chart recorder using Po-ne-mah Physiology Platform Software. The neurotransmitter 5 Hydroxytryptophane (5HT) (Sigma) at 130 μm, and atropine at 5-10 mM were used as controls. Atropine blocks the muscarinic effect of acetylcholine.

Varying doses of PROK2 from 1-400 ng/ml were tested for activity on strips of ileum. Muscle contractions were detected immediately after adding PROK2 protein and were recorded at concentrations as low as 1 ng/ml or 100 picomolar. The EC 50 of this response was approximately 10 ng/ml or 1 nM. PROK2 was tested for activity in the presence of 5HT, and a secondary contraction was observed. PROK2 was tested for activity in the presence of 0.1 μM tetrodotoxin (TTX), the nerve action potential antagonist and no reduction in the PROK2 effect was observed. PROK2 was also tested for activity in the presence of 100 nM Verapamil, the L-type calcium channel blocker. A significant reduction in the amplitude of the contractile response was observed.

Results of the effect of PROK2 on contractions in the ileum are shown in Table 10.

TABLE 10
Summary of Ileum Organ Bath Test Results

| Treatment | Ileum |
|---|---|
| 40 ng/ml PROK2 | +C |
| 40 ng/ml | +C |
| PROK2+130 μM | |
| 5HT | |
| 40 ng/ml PROK2+ | +C |
| 5 mM Atropine | |
| 40 ng/ml PROK2+ | − |
| 1 μM Verapamil | |
| 40 ng/nL PROK2+ | +C |
| 0.1 μM TTX | |

+C = Contraction Observed
− = No PROK2 effect observed

Results of the effect of PROK2 on contractions in duodenum, jejunum, proximal colon, and distal colon were performed at a concentration of 40 ng/ml did not produce contractions in duodenum, jejunum, or distal colon. However, relaxation of the tissue of the proximal colon was observed when the same concentration of PROK2 was added.

### Example 19

### Effect of Dose on Contractility in Guinea Pig Ileal Organ Bath Assay

All intestinal sections from the guinea pig ileum were obtained and tested using the same protocol and reagents as described in Example 6. Longitudinal strips of guinea pig ileum were mounted in the organ bath and allowed to stabilize for approximately 20 minutes. Acetylcholine (ACH) at a concentration of 10 μg/ml was added to tissue to confirm contractile activity. Two flush and fill cycles were run to wash ACH from the intestinal tissue. Baseline activity was confirmed for approximately 25 minutes. PROK2 was added to the organ bath at a final concentration of 1.0 ng/ml and an approximate 0.5 gram of deflection was recorded. The 1.0 ng/ml PROK2 dose was left on the tissue for 5 minutes to allow the tissue to return to baseline levels, and then a 10 ng/ml dose was added. Another contractile response was noted that resulted in a 2.0 gram deflection. The 10 ng/ml dose was left on for another 5 minutes before dosing the tissue with a 20 ng/ml dose of PROK2. Another contractile response was observed, yielding an approximate 2.2 gram deflection. Following a 5 minute incubation, the tissue was treated with a 40 ng/ml dose of PROK2. The tissue contracted again, with an approximate 2.0 gram deflection. The highest response was observed at the 20 ng/mL PROK2 dose.

### Example 20

### Effect of PROK2 on Gastric Emptying and Intestinal Transit

Eight-week old female C57B1/6 mice were fed a test meal consisting of a methylcellulose solution or a control, and both gastric emptying and intestinal transit was measured by determining the amount of phenol red recovered in different sections of the intestine. The test meal consists of a 1.5% aqueous methylcellulose solution containing a non-absorbable dye, 0.05% phenol red (50 mg/100 ml Sigma Chemical Company Catalogue # P4758). Medium viscosity carboxy methylcellulose from Sigma (Catalogue #C4888) with a final viscosity of 400-800 centipoises was used. One group of animals was sacrificed immediately following administration of test meal. These animals represent the standard group, 100% phenol red in stomach or Group VIII. The remaining animals were sacrificed 20 minutes post administration of test meal. Following sacrifice, the stomach was removed and the small intestine was sectioned into proximal, mid and distal gut sections. The proximal gut consisted approximately of duodenum, the mid gut consisted approximately of duodenum and jejunum, and the distal gut consisted approximately of ileum. All tissues were solubilized in 10 mls of 0.1 N NaOH using a tissue homogenizer. Spectrophotometric analysis was used to determine the OD and hence the level of gastric emptying and gut transit.

Each treatment group consisted of 10 animals, except for the animals being used as a standard group and the caerulein control group where the n=5. The study was broken down into two days, such that one half of all treatment groups are done on two consecutive days. The animals were fasted for 18 hrs in elevated cages, allowing access to water. The average weight of the mice was 16 grams.

Baculovirus-expressed PROK2 protein with a C-terminal Glu-Glu tag formulated in 20 mM MES buffer, 20 mM NaCl, pH 6.5 was diluted into 0.9% NaCl+0.1% BSA using siliconized tubes. (Sigma sodium chloride solution 0.9%, and Sigma BSA 30% sterile TC tested solution, Sigma Chemical Co, St Louis, Mo.). The protein concentration was adjusted so as to be contained in a 0.2 ml volume per mouse. Vehicle animals received an equivalent dose of PROK2 formulation buffer based on the highest (775 ng/g) treatment group.

Treatments were administered in a 0.2 ml volume via IP (intraperitoneal) injection two minutes prior to receiving 0.15 ml phenol red test meal as an oral gavage. Twenty minutes post administration of phenol red, animals were euthanized and stomach and intestinal segments removed. The intestine was measured and divided into three equal segments: proximal, mid and distal gut. The amount of phenol red in each sample was determined by spectrophotometric analysis and expressed as the percent of total phenol red in the stomach (Group VIII). These values were used to determine the amount of gastric emptying and gut transit per tissue collected. The CCK analogue caerulein at 40 ng/gram was used as a positive control and was administered five minutes prior to gavage, at which concentration it inhibits gastric emptying. Colormetric analysis of phenol red recovered from each gut segment and stomach was performed as follows. After euthanization, the stomach and intestinal segments were placed into 10 mls of 0.1 N NaOH and homogenized using a polytron tissue homogenizer. The homogenate was incubated for 1 hour at room temperature then pelleted by centrifugation on a table top centrifuge at 150×g for 20 minutes at 4 degrees C. Proteins were precipitated from 5.0 mls of the homogenate by the addition of 0.5 ml of 20% trichloracetic acid. Following centrifugation, 4 mls of supernatant was added to 4 mls of 0.5 N NaOH. A 200 μl sample was read at 560 nm using Molecular Devices Spectra Max 190 spectrophotometer. The amount of gastric emptying was calculated using the following formula: percent gastric emptying=(1−amount phenol red recovered from test stomach/average amount of phenol red recovered from Group VII stomach)×100. The amount of gastric transit was expressed as the percent of total phenol red recovered.

Results are shown in Table 7, below. Since test meal was not detected in the distal gut under any conditions, these data are not included. As expected, caerulein at 40 ng/ml inhibited gastric emptying (93.8% of test meal in stomach after 20 minutes compared to 63.8% with vehicle). Consistent with inhibited gastric emptying, in the caerulein treated group only 2.6% of meal was measured in the proximal gut and 1.2% in the mid gut.

At the lowest PROK2 concentration, 0.78 ug/kg body weight, a slight increase in gastric emptying compared to vehicle was observed (56.3% of meal remaining versus 63.8% with vehicle). Consistent with an increase in gastric emptying, increased meal was detected in the proximal gut of the PROK2 treated animals compared to vehicle control, 25.5% and 18.4% respectively. At the 7.8 ug/kg dose, PROK2 treated animals had 20% less test meal in the stomach (p=0.001), 16.6% more meal in the proximal gut (p=0.004) and 3.5% more meal in the mid gut. The largest effect was observed with the 77.5 ug/kg animals where gastric emptying was increased approximately 2 fold (37.8% test meal in PROK2 treated animals and 63.8% in vehicle treated animals p=0.0002). Intestinal transit was also increased significantly as a greater than 2 fold increase in test meal in the mid gut was measured in the PROK2 treated animals over vehicle control (37.1% compared to 15% (p=0.004). At the final, 775 ug/kg dose, increased gastric emptying was detected over control 46.6% compared to 63.8%, but the effect was not as great as the 77.5 μg/kg dose. Increased intestinal transit was detected in the mid gut (26% versus 15%), but the effect was not as significant as that observed with the lower 77.5 ug/kg dose. These data suggest that at higher concentrations, PROK2 can inhibit gastric emptying and intestinal transport.

TABLE 11
Description of treatment groups and results

| | Number of | % Test Meal | % Test Meal in | % Test Meal |
|---|---|---|---|---|
| Treatment Groups | Animals | in Stomach | Proximal Gut | in Mid Gut |
| Group I Vehicle (Buffer | N = 10 | 63.8% ± 3.8% | 18.4% ± 2.4% | 15% ± 3.3% SE |
| for PROK2) | | SE | SE | |
| Group II PROK2 | N = 10 | 56.3% ± 5.2% | 25.5% ± 4.1% | 14.6% ± 4% SE |
| 0.78 μg/kg body weight | | SE | SE | |
| *Group III PROK2 7.8 μg/kg | N = 10 | 43.7% ± 3.2% | 35.0% ± 5.4% | 18.5% ± 5.1% |
| body weight | | SE | SE | SE |
| | | *p = .001 | *p = .004 | |
| *Group IV PROK2 | N-10 | 37.8% ± 4.5% | 26.6% ± 5.1% | 37.1% ± 7.1% |
| 77.5 μg/kg body weight | | SE | SE | SE |
| | | *p = .0002 | | *p = .004 |
| *Group V PROK2 775 μg/kg | N = 10 | 46.6% ± 4.5% | 24.0% ± 5.9% | 26% ± 4.3% SE |
| body weight | | SE | SE | *p = .05 |
| | | *p = .009 | | |
| Group VI Caerulein (CCK | N = 10 | 93.8% ± 1.0% | 2.6% ± 0.9% | 1.2% ± 0.3% |
| analogue positive control) | | SE | SE | SE |
| 40 ng/g body weight | | | | |
| Group VII Sham non- | N = 5 | 100% | NA | NA |
| treated | | | | |

### Example 21

PROK2 Activity in Organ Bath

Organ bath testing was also performed with PROK2 using at a variety of tissues obtained from guinea pigs. A force transducer was used to record the mechanical contraction using IOX software (EMKa technologies, Falls Church, Va.) and Datanalyst software (EMKa technologies, Falls Church, Va.). Tissues analyzed included: duodenum, jejunum, ileum, trachea, esophagus, aorta, stomach, gall bladder, bladder and uterus.

### A. Organ Bath Methods

Two month old male guinea pigs (Hartley, Charles River Labs) weighing 250 to 300 g were fasted with access to drinking water for ˜18 hours then euthanized by CO₂ asphyxiation. All tissues were rinsed with Krebs buffer (1.2 mM MgSO₄, 115 mM NaCl, 11.5 mM glucose, 23.4 mM NaHCO₃, 4.7 mM KCl, 1.2 mM NaH₂PO₄, and 2.4 mM CaCl₂, oxygenated with 95% O₂-5% CO₂, pH 7.4, temperature 37° C.) then suspended in the 5 ml organ bath and pre-tensioned. All tissues were tested with positive controls to establish their viability prior to running. Positive controls used were CCK-8, acetylcholine (ACH), histamine, or 5HT, and were purchased from Sigma (Saint Louis, Mo.). All tissues were treated with a vehicle control, phosphate buffered saline (PBS), to rule out the possibility of vehicle effects.

1) Tissues that did not give a response to PROK2 in the organ bath:
Tracheal ring: 3 mm wide tracheal ring (3 cm away from brachial branches) was collected and allowed to equilibrate at 5 gram tension prior to any treatments. The positive control was 20 ug/ml ACH, which gave an approximate 1 gram deflection. No effect seen with PROK2 at 80 ng/ml.
Aortic ring: 3 mm wide aortic ring (immediately adjacent to aortic arch) was collected and allowed to equilibrate at 4 gram tension prior to any treatments. The positive control was 2 mg/ml KCl, which gave an average one gram deflection. PROK2 at 80 ng/ml did not cause a visible effect.
Esophagus: 2 cm in length esophagus (2 cm away from cardia) was suspended and allowed to equilibrate at 1 gram tension prior to any treatments. Two mg/ml 5HT gave an approximate 1.4 grams deflection. PROK2 at 20 ng/ml had no visible effect.
Gall bladder: Lumenal fluid was aspirated out with 1 ml syringe then longitudinally suspended and allowed to equilibrate at 1 gram tension prior to any treatments. Five ng/ml of ACH gave a 0.4 gram deflection response. No effect was seen with 20 ng/ml PROK2.
Bladder: 1.5 cm×0.3 cm longitudinal strip was suspended and allowed to equilibrate to 0.5 gram tension prior to any treatments. Positive controls induced a contractile response, but no activity was seen at a 80 ng/ml PROK2 dose.

2) Tissues that responded to PROK2:
Stomach/antrum: 1.5 cm×0.3 cm longitudinal strip was suspended and allowed to equilibrate to 0.5 gram tension prior to any treatments. Treatment with either 5 ng/ml ACH or 80 ng/ml CCK 8 resulted in an approximate one gram deflection. Eighty ng/ml PROK2 also produced a contractile response of approximately 0.5 gm deflection.
Duodenum: 2 cm in length duodenum (2 cm away from pylorus) was suspended and allowed to equilibrate at 1 gram tension prior to any treatments. ACH gave an approximate 0.75 gm deflection. Twenty ng/ml PROK2 also gave a contractile response of approximately 0.5 grams deflection.
Jejunum: 2 cm in length jejunum (midpoint between pylorus and ileal-cecal junction) was suspended and allowed to equilibrate at 1 gram tension prior to any treatments. ACH gave an approximate 1.0 gram deflection and 20 ng/ml PROK2 gave an approximate 0.5 gram deflection contractile response.
Ileum: 8 cm in length ileum (2 cm away from ileal-cecal junction) was collected and flushed with Krebs buffer to remove any fecal debris if present then cut into four equal pieces. All tissues were suspended and allowed to equilibrate at 1 gram tension prior to any treatments. The ileum was run at the same time to compare PROK2 effects on the small intestine. ACH gave an approximate 1.5 gram deflection, and 20 ng/ml PROK2 also gave a 1.5 gram deflection.
Proximal Colon: 2 cm in length colon (2 cm away from cecum) was suspended and allowed to equilibrate at 0.5 gram tension prior to any treatments. PROK2 at 20 ng/ml induced a relaxation effect with a decrease in muscle tone and a decrease in the amplitude of the contractions.

PROK2's contractile effects are specific to the gastrointestinal tract. The greatest contractile response is seen in the ileum, with lesser contraction seen in the duodenum, jejunum, and antrum. The relaxation effect in the proximal colon is suggestive of a coordinated effect on gut motility. As the smooth muscle contraction is enhanced in the antrum and the small intestine, the large intestine is preparing to accommodate the approaching meal by relaxing. Coordinated contractile activity between different parts of the gut will result in improved gastrointestinal function.

### Example 22

### Comparative Activity of PROK2 and PROK1 in the Organ Bath

Both PROK2 and PROK1 have contractile effects on intestinal tissue in the organ bath. Side by side comparisons were made to compare activity in tissue derived from the same animal.

Ileal strips from guinea pig were tested for contractility using methods described above. PROK1 was purchased from PeproTech Inc. (Rocky Hill, N.J.). Activity was compared at 40, 12, and 3 ng/ml concentrations. ACH at 5 ng/ml was used as a positive control. Contractile responses were normalized to the ACH response in each tissue. All three doses were run on separate ileal longitudinal tissue strips obtained from the same animal.

Results: Contractile effects were normalized to the ACH positive control and are expressed as the ratio of PROK2 or PROK1 to ACH in the table below.

TABLE 12

| | | | PROK2 | | | PROK1 | |
|---|---|---|---|---|---|---|---|
| Conc (ng/ml) | ACH | PROK2 | PROK2:ACH | ACH | PROK1 | PROK1:ACH | |
| 40 | 1.26 | 1.28 | 1.02 | | 1.25 | 0.58 | 0.46 |
| 12 | 2.5 | 2.51 | 1.00 | | 2.26 | 0.61 | .027 |
| 3 | 1.38 | .047 | .034 | | 1.73 | .027 | .016 |

Conclusions: PROK2 is approximately twice as active as PROK1 when comparing contractility in the ileum.

### Example 23

### Synergistic Effects of PROK2 and PROK1 in Gastrointestinal Contractility

In order to determine the combined effects of PROK2 and PROK1 on contractile activity, ileal tissues were pre-treated with varying doses of PROK1, followed by increasing doses of PROK2.

All tissues are stabilized, treated with ACH, and again stabilized prior to pre-treatment with PROK1 at concentrations of 0.8, 3.0 or 12 ng/ml. PROK1 was left on tissue for approximately 20 minutes prior to dosing with 20 ng/ml PROK2.

Results: Large 3 gram deflection contractions with PROK2 were observed when the tissue was pre-treated with 0.8 ng/ml PROK1. These contractions were larger than what is normally observed with a 20 ng/ml dose of PROK2, where contractile effects of approximately 1.5 to 2.0 grams deflection are normally observed. PROK1 alone at 0.8 ng/ml has a negligible contractile effect.

Conclusions: These data suggest that by pre-treating with a low dose of PROK1, and then treating with PROK2, increased motility effects may be obtained.

### Example 24

### Effect of PROK2 in Post-Operative Ileus In Vivo

Five to 25 male Sprague-Dawley rats (˜240 g) per treatment group were used for these POI studies. Animals were fasted for ˜22-23 h (with 2 floor grids placed in their cages to prevent them from having access to their bedding) with free access to water. While under gas isoflurane anesthesia, the rat's abdomen was shaved and wiped with betadine/70% ethanol. A midline incision was then made through the skin and linea alba of the abdomen (3-4 cm long), such that intestines were visible and accessible. The cecum was manipulated for 1 min with sterile saline-soaked gauze, using a gentle, pulsatile-like pressure. This procedure was consistent from animal to animal in order to reduce inter-animal ileus variability. The linea alba was sutured with silk suture and the skin closed with wound clips. Animals were kept on water-jacketed heating pads during recovery from surgery and placed back into their cages once they regained full consciousness.

When fully conscious, rats were administered 1.0 ml of the test meal 15 minutes following completion of cecal manipulation (CM); one minute or 20 minutes later, rats were administered 0.8 or 5 ug/kg BW E. coli-produced PROK2, or saline/0.1% w/v/BSA via indwelling jugular venous catheter. PROK2 was diluted with saline/0.1% BSA to the desired concentration (based on average BW of rat [˜240 g] and a 0.1 ml injection volume for i.v.) immediately prior to study, using siliconized microfuge tubes.

The test meal consisted of 1.5% (w/v) aqueous methylcellulose solution (medium viscosity methylcellulose from Sigma 400 centipoises; catalog #M-0262) along with a non-absorbable dye, 0.05% (50 mg/100 ml) phenol red (Sigma catalog #P-4758; lot #120K3660). Twenty minutes following administration of the test meal, animals were anesthetized under isoflurane and sacrificed by cervical dislocation. The stomach and intestinal segments were removed, and the amount of phenol red in each segment was determined by spectrophotometric analysis (see below) and expressed as the percent of total phenol red recovered per rat. These values are used to determine the amount of gastric emptying and gut transit per tissue collected.

Colorimetric analysis of phenol red recovered from each gut segment and stomach were performed according to a modification of the procedure outlined by Scarpinato and Bertaccini (1980) and Izbeki et al (2002). Briefly, following euthanization, the stomach and intestinal segments were placed into 20 ml of 0.1 N NaOH and homogenized using a Polytron tissue homogenizer. The Polytron was then rinsed with 5 ml of 0.1 N NaOH and added to the previous 20 ml, along with another 15 ml of 0.1 N NaOH. Homogenate was allowed to settle for at least 1 hour at room temperature. Proteins were precipitated from 5 ml of the supernate by the addition of 0.5 ml of 20% trichloracetic acid. Following centrifugation (3000 rpm for 15 min), 1 ml of supernatant was added to 1 ml of 0.5 N NaOH. A 0.2 ml sample (in a 96-well plate) was read at 560 nm using Molecular Devices Spectra Max 190 spectrophotometer. The extent of gastric emptying and intestinal transit were expressed as percent of total phenol red recovered per rat.

Data indicated that PROK2 (0.8 and 5.0 ug/kg, i.v.) significantly increased gastric emptying and upper intestinal transit of this semi-solid, non-nutritive meal by approximately 1.6 to 2.-fold compared to emptying and transit observed in vehicle-treated rats. Efficacy in this model was observed when these doses of PROK2 are administered at either 1 min or 20 min following meal administration.

### Example 25

### Effect of i.v. and ip. BV- and E. Coli-Produced PROK2 on Gastric Emptying and Intestinal Transit of a Phenol Red Semi-Solid Meal in Rats

Male Sprague-Dawley rats (˜240 g) were used for this study, with 6-12 animals per treatment group. Animals were fasted for ˜24 h (with 2 floor grids placed in their cages to prevent them from having access to their bedding) with free access to water. One minute following the administration of 1.0 ml of test meal, rats were administered varying doses of PROK2 (0.01 to 30 ug/kg BW) or saline/0.1% w/v BSA via indwelling jugular venous catheter. For i.p. dosing, PROK2 (0.1 to 100 ug/kg BW) or saline/0.1% BSA was administered either 1 or 10 min prior to or 1 min after the meal. PROK2 was diluted with saline/0.1% BSA to the desired concentration (based on average BW of rat [˜240 g] and a 0.1 ml injection volume for i.v. or 0.5 ml injection volume for i.p.) immediately prior to study, using siliconized microfuge tubes. The test meal consisted of 1.5% (w/v) aqueous methylcellulose solution (medium viscosity methylcellulose from Sigma 400 centipoises; catalog #M-0262) along with a non-absorbable dye, 0.05% (50 mg/100 ml) phenol red (Sigma catalog #P-4758; lot #120K3660). Fifteen or 20 min following administration of the test meal, rats were anesthetized under isoflurane and sacrificed by cervical dislocation.

The stomach and intestinal segments were removed, and the amount of phenol red in each sample was determined by spectrophotometric analysis (see below) and expressed as the percent of total phenol red recovered per rat. These values were used to determine the amount of gastric emptying and gut transit per tissue collected.

Colorimetric analysis of phenol red recovered from each gut segment and stomach were performed according to a modification of the procedure outlined by Scarpinato et al Arch Int. Pharmacodyn. 246:286-294 (1980) and Piccinelli et al. Naunyn-Schmiedeberg's Arch. Pharmacol 279: 75-82 (1973). Briefly, following euthanization, the stomach and intestinal segments were placed into 20 ml of 0.1 N NaOH and homogenized using a Polytron tissue homogenizer. The Polytron was then rinsed with 5 ml of 0.1 N NaOH and added to the previous 20 ml, along with another 15 ml of 0.1 N NaOH. Homogenate was allowed to settle for at least 1 hour at room temperature. Proteins were precipitated from 5 ml of the supernate by the addition of 0.5 ml of 20% trichloracetic acid. Following centrifugation (3000 rpm for 15 min), 1 ml of supernatant was added to 1 ml of 0.5 N NaOH. A 0.2 ml sample (in a 96-well plate) was read at 560 nm using Molecular Devices Spectra Max 190 spectrophotometer. The extent of gastric emptying and intestinal transit were expressed as percent of total phenol red recovered per rat.

Gastric emptying and intestinal transit of this semi-solid meal were increased by approximately two-fold following i.v. administration of 0.1-1.0 μg/kg BW BV- or E. coli-produced PROK2. Inhibitory effects of gastric emptying and intestinal transit were observed using higher doses (10-100 ug/kg BW for i.p. dosing; 30 ug/kg BW for i.v. dosing) of BV- and E. coli-PROK2. The inhibitory observations were especially evident when these higher doses of PROK2 were administered i.v. at 1 minute following test meal administration, or when administered i.p. at 10 minutes prior to test meal administration. Similar results were observed when PROK1 was administered i.v. at 30 μg/kg.

### Example 26

### Effect of i.v. BV- and E. Coli-Produced PROK2 on Gastric Emptying and Intestinal Transit of a Phenol Red Semi-Solid Meal in Mice

Female C57B1/6 mice, 8 to 10 weeks old, were used for the study, which consisted of eight treatment groups and ˜9 mice per group. The animals were fasted for ˜20 hrs in cages containing floor screens, and allowed access to water. Animals were weighed to determine proper dose, and their average weight was used to adjust the protein concentration. PROK2 protein (in stock solutions of either 20 mM Mes buffer/20 mM NaCl pH 6.5; or in PBS, pH 7.2) dilutions were prepared in siliconized tubes just prior to injections. Doses were based on the average weight of the study animals (approximately 20 g) and adjusted with saline 0.1% w/v BSA to 0.1 ml injection volumes per mouse. PROK2 and vehicle treatments were administered via i.v. tail vein injection 1-2 minutes prior to receiving 0.15 ml phenol red test meal as an oral gavage. The test meal consisted of 1.5% w/v aqueous methylcellulose solution (medium viscosity carboxy methylcellulose from Sigma with a final viscosity of 400-800 centipoises; catalog #C-4888; lot #108H0052) containing a non-absorbable dye, 0.05% phenol red (Sigma catalog #P-4758; lot #120K3660). Twenty minutes post-administration of the test meal, animals were euthanized and stomach and intestinal segments removed. The small intestine was measured and divided into three equal segments: proximal, mid and distal gut. The amount of phenol red in each sample was determined by spectrophotometric analysis (as described above for in Examples 20 and 21) and expressed as the percent of total phenol red recovered per mouse. These values were used to determine the amount of gastric emptying and gut transit per tissue collected.

Results indicated that there were increases in gastric emptying and intestinal transit in mice treated with i.v. PROK2 at doses ˜1-10 ug/kg BW. Trends toward inhibition of gastric emptying and intestinal transit were observed using higher doses (>50 ug/kg i.v. in mice) of PROK2.

### Example 27

### Effect of BV- and E. coli-produced PROK2 on Gross Morphology of Stomach and Intestines of Urethane-Anesthetized Rats

Studies were conducted in urethane-anesthetized male Sprague-Dawley rats to determine whether i.v. administration of BV- or E. coli PROK2 (doses up to and including 30 ug/kg BW; a dose known to induce intestinal motility) affected the gross appearance of the stomach and small intestine.

Rats were fasted (with access to water) on double floor grates in clean cages for ˜19 h. Between 07:00 and 08:30 am, rats received an i.p injection of urethane (0.5 ml/100 g BW of a 25% solution) and had a jugular venous catheter inserted. Anesthetized rats were returned to their cages and kept on warming pads (maintained at 37° C.) throughout the day, with additional i.p. doses of urethane administered as needed. An appropriate level of anesthesia was monitored using the toe-pinch reflex test.

At ˜5 minute intervals between animals saline was administered via the jugular vein, followed by either vehicle (PBS) or BV- or E. coli-produced PROK2 at increasing doses (3, 10 and 30 ug/kg BW; 0.1 ml injection volume) every hour for 3 hours (total of 43 ug/kg BW). PROK2 protein dilutions were prepared just prior to injection. Dose was based on the weight of the study animal (approximately 225 grams) and adjusted so that it was contained in 0.1 ml total volume of diluent (saline/0.1% BSA). Protein was diluted using siliconized microfuge tubes. Rats also received infusions of saline via Harvard pumps at a rate of 0.5 ml per hour. Approximately 8-9 hours later following the initial dose of urethane, rats were sacrificed by cervical dislocation (under anesthesia) and their stomachs and small intestine removed for inspection and morphological evaluation.

There was no evidence of gastric or intestinal lesions in any of the rats. A vehicle-treated rat had some dark fluid within a small segment of the intestinal lumen; there was not any dark fluid observed in the PROK2-treated rats. There was a significant amount of mucous within the intestinal lumen in all treatment groups, most likely as a result of the urethane anesthesia and fasting protocol.

### Example 28

### Effects of B V-Produced PROK2 on In Vivo Gastrointestinal Contractility in Anesthetized Experimental Mammals

“Sonomicrometry” is a technique, which utilizes piezoelectric crystals to measure gastrointestinal distensibility, compliance, and tone in vivo (Sonometrics, Corp. Ontario, Canada). Crystals can be placed anywhere along the gastrointestinal tract in experimental mammals. Peristaltic and segmentation contractions in the stomach and/or intestine can then be accurately quantified and qualified with great detail in response to the administration of PROK2. This system offers a great deal of detailed and sophisticated outcome measures of intestinal motility/contractility.

This method of digital ultrasonomicrometry was used to investigate motility and/or contractility in the ileum, jejunum, cecum and proximal colon as described by Adelson et al. Gastroenterology 122, A-554. (2002) in ten rats (two groups of 5 male Sprague-Dawley rats) following an i.v. infusion of the vehicle (saline/0.1% w/v BSA) and escalating doses of BV-produced PROK2. For these experiments, piezoelectric crystals were attached using a small drop of cyanoacrylate glue (Vetbond, 3M Animal Care, St. Paul, Minn.) to the relevant intestinal locations. After laparatomy the urethane anesthetized rats were maintained at 37° C. via a feedback-controlled heater. Sonometric distance signals were acquired continuously at a rate of 50 samples/sec via a digital sonomicrometer (TRX-13, Sonometrics Corp, London ONT) connected to a Pentium III class computer running SonoLAB software (Sonometrics Corp, London, Ontario, Canada). Digitally-acquired distance data were simultaneously recorded as analog signals via an installed 4-channel DAC. These sonometric analog signals, along with all analog physiological data (rectal temperature, blood pressure, EKG, respiratory rate) were acquired using a Microl401 A/D interface (Cambridge Electronic Design, Ltd, Cambridge) connected to a Pentium II class computer running Spike 2 (Cambridge Electronic Design, Ltd, Cambridge) data acquisition software to allow real-time observation and analysis of experiment progress. This method allows simultaneous observation of distance measurements for 4 crystal pairs. Baseline levels were obtained between each vehicle and PROK2 infusion. Both circular and longitudinal motion were monitored using triads of piezoelectric crystals 1 mm in diameter (Sonometrics Corp.) affixed so that two of the three were oriented parallel to the longitudinal axis and the third was oriented to the perpendicular axis.

Motility responses to applied stimuli may comprise tonic and/or phasic components. Tonic and phasic components of responses were analyzed separately. The tonic component of the trace was obtained by replacing each point in the trace with the median value of the trace over the surrounding 10 s. The phasic component was obtained by applying to the original trace the inverse operation of a smoothing function with a 10 s window, i.e. by removing the ‘DC component’ with a time constant of 10 s. Tonic responses were analyzed in terms of mean value during a response, 1-min maximum excursion from baseline, duration of response, and integrated response (mean normalized response times duration). Phasic activity was analyzed in terms of its rate and amplitude. Changes in relationships between motility in different gut regions measured simultaneously were analyzed using cross-correlation of continuous signals and event correlations of peak positions.

Strong contractility responses were observed in the ileum of PROK2-treated rats at i.v. doses as low as 3 ug/kg BW; contractions were also noted in the jejunum and duodenum, though not as strong as those observed for the ileum. Responses associated with a relaxation were observed in the proximal colon.

### Example 29

### Effects of ip. Administration of B V-Produced PROK2 on Distal Colonic Transit in Conscious Mice

Adult male C57/BL6 mice (6-8 weeks of age; Harlan, San Diego, Calif.) were used for this study with 6-10 mice per treatment group. Mice were maintained on a 12:12-h light-dark cycle with controlled temperature (21-23° C.) and humidity (30-35%), and were group housed in cages with free access to food (Purina Chow) and tap water. Mice were deprived of food for 18-20 h, with free access to water before the experiments. BV-produced PROK2 in stock solution of 20 mmol MES and 20 mmol NaCl at pH 6.5 was stored at −80° C. On the day of the experiment, PROK2 was diluted to 0.9% NaCl with 0.1% BSA. The pH for both vehicle and PROK2 at various doses was 6.5.

Distal colonic transits were measured as previously described (Martinez V, et al. J Pharmacol Exp Ther 301: 611-617(2002.)). Fasted mice had free access to water and pre-weighed Purina chow for a 1-h period, then were briefly anesthetized with enflurane (1-2 min; Ethrane-Anaquest, Madison, Wis.) and a single 2-mm glass bead was inserted into the distal colon at 2 cm from the anus. Bead insertion was performed with a glass rod with a fire-polished end to avoid tissue damage. After bead insertion the mice were placed individually in their home cages without food and water. Mice regained consciousness within a 1-2 min period and thereafter showed normal behavior. Distal colonic transit was determined to the nearest 0.1 min by monitoring the time required for the expulsion of the glass bead (bead latency).

At the end of the 1 h feeding period, mice were briefly anesthetized with enflurane for bead insertion into the colon followed by the intraperitoneal injection of either vehicle, or PROK2 (3, 10, 30, or 100 μg/kg). Animals were returned to their home cages without food or water and the bead expulsion time was monitored. Results were expressed as Mean ±S.E. and analyzed using one-way ANOVA.

In mice, fasted for 18-20 h, re-fed for 1 h, PROK2 injected i.p. (3, 10, 30, and 100 μg/kg) showed no significant changes in bead expulsion time in response to the i.p. injection of BV-PROK2 (3, 10 and 30 μg/kg): 32.7±6.1, 23.1±4.5 and 34.2±5.6 min respectively compared with 21.1±3.9 min in i.p. vehicle injected group. In a second group of mice, treated similarly except administered higher doses of BV-PROK2, the measurement of distal colonic transit showed a dose-related tendency to increase the time at which the bead is expelled in response to the i.p. injection of BV-PROK2 (30 and 100 μg/kg) (29.8±7.8 and 35.1±3.7 min respectively compared with 22.3±5.7 min after i.p. injection of vehicle) although changes did not reach statistical significance.

### Example 30

### Preparation of Hybridomas

### Immunization:

A group of five 6-8 week old female BALB/c mice were immunized with a purified, recombinant version of human PROK2 that had been produced in E. coli. Before use as an immunogen this molecule was first conjugated to keyhole limpet hemocyanin (KLH) and it was estimated that PROK2 comprised approximately 30% of the mass of the conjugate (PROK2-KLH). The mice were immunized by intraperitoneal injection with 75 ug of the conjugate in combination with Ribi adjuvant (containing CWS) according to manufacturer's instructions on days 1, 14, 28 and 51. Seven to ten days after the third and fourth immunizations and about 36 days after the fourth immunization the mice were bled via the retroorbital plexus and the serum separated from the blood for analysis of its ability to inhibit the binding and subsequent stimulatory activity of human PROK2 to a cell line transfected with the human PROK2 receptor. The sera were also analyzed for their ability to bind to PROK2 bound to a polystyrene ELISA plate and their capacity to bind to PROK2 in a solution phase assay. Mice chosen to be spleen/lymph node donors for fusion were given a final injection, via intravascular injection, of 10 ug of PROK2 in PBS on days 100 and 101.

### Fusion:

Three days after the last intravascular immunization with PROK2 the spleen and lymph nodes from these mice were harvested, combined, processed into a single cell suspension (total of 2.925×108 cells) and then fused to a clone of the mouse myeloma cell line P3-X63-Ag8.653 (Kearney, J. F. et al., J. Immunol. 123:1548-50, 1979)(designated P3-X63-Ag8.653.3.12.11) at a 2:1 lymphoid cell:myeloma cell ratio with 2.4 mL PEG 1450 for 3 minutes using standard methods known in the art (Lane, R. D. J Immunol Methods 81:223-8, 1985).

### Fusion Protocol:

The fusion was performed according to the following procedure:

Preparation of 50% PEG

Materials: 1) PEG 1450 [Acros, cat. # 41804-1000 (100 g bottle), cat. # 41804-5000 (500 g bottle)]; 2) Stoppered glass vial (Kimble Glass Inc., 27×55 mm, 5 dram, cat. # 60975L-5); 3) Phosphate buffered saline (PBS, pH 7.4, GIBCO/Invitrogen Corp., cat. # 10010-023); 4) 22 gauge needle (Becton-Dickinson, 22G1½, cat. # 305156); 5) DMSO (Sigma, cat. # D-5879); 6) 10 mL Luer-Lok syringe (Becton-Dickinson cat. # 309604); 7) Sterile 0.22 um filter (Millipore, Millex-GP, cat. # SLGP R25K₅ or Gelman Sciences, cat. # 4192); 8) 14 mL snap cap PP tube (Falcon 352059); 9) Foil; 10) Water bath (37° C.) and small glass beaker (100 mL).

### Procedure:

1) Weigh out slightly more than 3 grams of PEG into glass vial. Record weight.

Calculate the amount of PBS (x) to add to vial according to the following proportion:

25 grams PEG=Weight of PEG

22.5 mLs PBS x mLs PBS

3) Cap the vial, place a 22 gauge needle through the cap to allow for air expansion and place vial in a small beaker of water in a 37° C. water bath. After PEG has dissolved in the PBS, swirl the vial well to ensure that the contents are well mixed.

4) Remove vial from water bath and remove needle from vial. Remove cap and add DMSO to 1/10th (v/w) of the weight of the PEG in the vial. Mix well by swirling. Filter PEG solution through a 0.22 um filter into a 14 mL snap cap tube. Snaphthe cap down completely. Cover tube below cap with foil and place tube back in a small beaker of water in a 37° C. water bath.

### Fusion:

Materials:

1) Culture Medium for Myeloma/Hybridoma Cells
Iscove's modified Dulbecco's medium (IMDM—cat.# 12440-053, GIBCO/Invitrogen Corp.)
Fetal clone I serum (cat.# SH30080.03, HyClone Laboratories) (non-heat-inactivated)
100× (2 mM) L-glutamine (cat.# 25030-081, GIBCO/Invitrogen Corp.)
100× (10,000 U/mL:10,000 ug/mL) penicillin G sodium: streptomycin sulfate (cat.# 15140-122, GIBCO/Invitrogen Corp.)

Put the above components together as follows:

a) Add 50 mLs of serum to a 500 mL bottle of IMDM

b) Add 5.6 mLs of 100×L-glutamine

c) Add 5.6 mLs of 100× pen-strep

Effective concentration of serum in this media will be 8.91% (v/v) and 1× for the other components.

This medium is referred to hereafter as complete IMDM medium. Store at 4° C. and use at 37° C.

2) Lymphocyte Preparation Medium (LPM)
Iscove's modified Dulbecco's medium (IMDM—cat.# 12440-053, GIBCO/Invitrogen Corp.)
100× (10,000 U/mL:10,000 ug/mL) penicillin G sodium: streptomycin sulfate (cat.# 15140-122, GIBCO/Invitrogen Corp.)

Add 5.05 mLs of 100× pen-strep to the IMDM for an effective 1× pen-strep final concentration.

Store at 4° C. and use at room temperature.

3) Fusion Medium

Culture medium for myeloma/hybridoma cells (complete IMDM medium) plus:
10% (v/v) hybridoma cloning factor (BM Condimed H1, Roche Diagnostics, cat. # 1088947), alternatively 5% (v/v) hybridoma cloning factor (Origen, Igen, cat. # 210001)
50×HAT (cat. # 25-046-CI, Mediatech/Cellgro) diluted to 1×

4) Sterile 50 mL centrifuge tubes (Falcon, cat. # 352070)

5) Aspiration set-up

6) Sterile 35 mm plastic petri dishes (Falcon, cat. # 353004)

7) Scalpel (Bard-Parker #4)

8) Scalpel blade (Bard-Parker, #20 Rib Back, carbon steel surgical blade, sterile)

9) 5, 10, 25 and 50 mL sterile pipets

10) Curved forceps

11) Frosted end microscope slides (VWR, cat. #48312-002) or (Mercedes Medical, cat. # 7760/90□). Sterilize by wiping (wetted gauze works well) or spraying all but the unfrosted end of the slide that you hold between your index finger and thumb (both sides) with 70% alcohol. Air dry the slide to completion by continuing to hold the slide in the laminar flow hood.

12) Sterile 40 um cell strainers (Falcon, cat. # 352340)

13) Sterile 1 mL plastic syringe (Becton-Dickinson, cat. # 309602)

14) Table top centrifuge

15) Sterile 14 mL snap-cap polypropylene (PP) tubes (Falcon, cat. # 352059)

16) 2% acetic acid in water

17) Hemocytometer with coverslip

18) Inverted microscope

19) Sterile, flat-bottomed 96-well plates (Costar, cat. #3596)

20) Sterile 250 centrifuge tubes (Corning, cat. # 430776)

21) 1 mL pipetman and sterile tips

22) 200 ul pipetman and sterile tips

23) 20 gauge needles (Becton-Dickinson, 20G1, cat. # 305175)

24) Sterile 24-well plates (Falcon, cat. # 353047)

25) 600 mL PP beaker

26) Sterile 50 mL polystyrene reagent reservoir [(Costar, cat. # 4870, 5 pack) or (VWR, cat.29442-474, single unit)]

27) Electronic multi-channel pipettor and tips (Thermo Labsystems, cat. # 0002206 060, 1500 uL model)

### Procedure:

1) Preparation of mouse myeloma (P3-X63-Ag8.653.3.12.11) cells.

Cells are grown in complete IMDM medium. They should be in log phase growth at the time of fusion. To achieve this, cells are split (1:4-1:5) every other day a week before fusion and usually 1:2 or 1:3 the day before fusion. Ideally, the myeloma cells should be at a density of 2-4×10⁵ cells/mL at the time of fusion. Have 500 mLs on hand the day of fusion.

Prior to obtaining spleen and lymph nodes from immunized mice, check flasks of myeloma cells to make sure the cells are in good shape and there are no signs of any contamination.

2) Euthanize designated animal(s) and aseptically remove spleen and any accessible lymph nodes. Place these in a 50 mL centrifuge tube containing 15-20 mLs sterile lymphocyte prep medium.

3) Aspirate all but 5-10 mLs of the media in the tube containing the spleen/lymph nodes. Swirl the tube to suspend the lymphoid organs and pour all into a 35 mm petri dish.

4) Prepare a single cell suspension of spleen and lymph node cells. Begin with lymph nodes. In another 35 mm petri dish containing 10 mLs of LPM, pre-wet the frosted end of a sterile microscope slide with LPM and place nodes with sterile forceps on this area. Using a scalpel with blade, cut the nodes into pieces (try for 2-4 per node). Pre-wet the frosted end of another sterile microscope slide and place this end over that of the other slide containing the cut up nodes, frosted face to frosted face. Make sure the nodes sit in a small puddle of LPM. Gently press the frosted ends of the two slides towards each other and with a circular motion, slide the nodes between the slides to liberate the lymphocytes. Try not to rub glass on glass. Continue this motion until only the lymph node stroma is left. Re-wet the slides in the media of the petri dish to remove cells and lymph node stroma.

Proceed next with the spleen in the same manner as was used for the nodes, except make many more cuts in this organ with the scalpel. Make cuts perpendicular to each other across the organ so that it looks like it has been diced into small pieces. Liberate WBC and RBC as above with frequent exchanges of media from the dish below. Discontinue this operation when there is no more red color remaining in the stromal tissue. Rinse off slides into the dish below with approx. 5 mLs of LPM.

5) Fill a 50 mL centrifuge tube with LPM. Place a 40 um cell strainer in the top of another 50 mL centrifuge tube. Pipet the contents of the petri dish to resuspend cells well and loosen cells from stromal components. Draw up approximately 8-9 mLs of fine cell suspension (leaving larger stromal pieces behind) and pass this suspension through the filter. Using the same pipet, go back to reload with fresh LPM (about 10 mLs) from the 50 mL tube. Repeat resuspension of cells in petri dish and transfer approximately 10 mLs to cell strainer. Continue these steps until approximately 45 mLs of strained cell suspension has been collected in the centrifuge tube. By this time the petri dish should be well rinsed out of cells with only larger stromal pieces left. There should be some spleen material (besides splenic stroma) on the filter mesh that was not completely suspended by the slide operation. Press this material through the mesh with the black rubber end of a plunger from a sterile 1 mL syringe. Wash liberated cells through mesh with 5 mLs of LPM.

6) Centrifuge cell suspension at 1100 RPM (Beckman Allegra 6 centrifuge) for 10 min. at RT.

7) Aspirate supernatant leaving approximately 200 uL behind to resuspend the pellet by shaking/tapping the centrifuge tube. Add 25 mLs fresh LPM to tube and gently resuspend the cells 2-3× with the pipet. Re-filter through another 40 um cell strainer in the top of a new 50 mL centrifuge tube. Wash filter with an additional 5 mLs of LPM. Place 360 uL of LPM in a 14 mL snap-cap polypropylene tube. After resuspending the WBC/RBC mixture, remove 40 uL and add to the tube containing 360 uL LPM for an effective 10-fold dilution.

8) Mix the 400 uL WBC/RBC suspension well by tapping the side of the tube to create a gentle vortex. Add 40 uL of 2% acetic acid to a 14 mL PP snap cap tube. Add 40 uL of the diluted cell suspension to the same tube, mix the contents well by shaking the tube, withdraw 40 uL and place on a hemocytometer. Count viable cells on an inverted microscope. Calculate the total number of WBC in the original 50 mL WBC/RBC suspension tube.

9) Calculate the number of myeloma cells that will be needed to affect a 2:1 spleen/lymph node cell:myeloma ratio fusion. Mix a flask of the myeloma cells well and pour into 50 mL tubes. Pipet the cells in one tube a few times with a 25 mL pipet to break up any clusters then remove a sample and add to a hemocytometer. Count viable cells on an inverted microscope. Calculate the number of mLs of myeloma suspension needed for the fusion.

10) Centrifuge the needed volume of myeloma cells at 1000 RPM (Beckman Allegra 6 centrifuge) for 5 minutes. Aspirate media leaving approximately 200 uL behind to resuspend the pellet by shaking/tapping the centrifuge tube. Add 20 mLs fresh LPM to the first tube, gently resuspend the cells 1× with the pipet and transfer contents to next tube. Continue this operation until all cells have been transferred to last tube and then add this suspension to the tube containing the WBC/RBC suspension. and mix. Rinse tubes sequentially with another 5 mLs of LPM, add to tube containing other cells, cap tube and mix.

11) Centrifuge cell suspension at 1100 RPM (Beckman Allegra 6 centrifuge) for 10 min. at RT. During this centrifugation, prepare fusion media that the fusion products will be diluted in prior to plating in 96 well plates. Decide what the seeding density will be and calculate the volume needed to plate the fusion at 200 uL/well of a series of 96 well plates. Split the volume of media equally between 2-4 250 mL PP centrifuge tubes. Also set up inside the hood a PP beaker filled to within an inch of the top with water at approximately 40° C.

12) Aspirate supernatant from tube completely. Resuspend cells by tapping tube fairly hard against the backside of the window on the hood. Pellet must be completely broken up into a fine suspension. This could take a minute or two of tapping hard.

13) Place tube into the beaker with the cap loosely fitting over the opening of the tube. Retrieve PEG solution from water bath. Bring up desired amount of PEG into a 1 mL pipetman tip or a 2 mL pipet (for volumes >1 mL). Add PEG, drop by drop, to the tube of cells over a period of 45 seconds, swirling contents of the tube in the beaker water bath continuously. After completing addition of the PEG, swirl tube every 5-10 seconds for 120 seconds.

14) Fill a 50 mL pipet with 50 mLs of 37° C. myeloma culture media and immediately begin adding to the fusion tube, drop by drop with constant swirling of the tube. Add the first 5 mLs over the first 30 seconds, the second 10 mLs over the next 30 seconds and the remaining media over the last 30 seconds. Addition of media should ideally increase in a logarithmic fashion over the 90 second interval.

15) Cap the tube, mix the tube's contents very gently by inverting the tube 2-3 times and place in a 37° C. beaker water bath for 15 minutes. The tube should be immersed nearly to the cap in the beaker.

16) Centrifuge tube at 850 RPM for 5 minutes. Aspirate the media leaving approximately 200 uL behind to resuspend the pellet by gently shaking the centrifuge tube. Make sure entire pellet is evenly resuspended with no obvious large clusters of cells. With a 25 or 50 mL pipet, remove 10 mLs of media from each 250 mL tube containing fusion media and gently add to the fusion tube. Gently pipet the cells once or twice to evenly distribute cells and add back to 250 mL tubes, 10 mLs per tube.

17) Put all but one tube of the cell suspension back in the water bath. Mix remaining tube by rotating the tube end-over-end. Plate cell suspension at 200 uL/well in 96 well culture plates. When done with the first tube, retrieve second tube and repeat procedure, etc.

18) Feed plates by 20 gauge needle aspiration and replacement of fusion media (generally 200 uL/well). Feed plates 2-3 times depending on the titer of fused mouse's (mice) serum on relevant antigen (generally days 5 and 8 for 2 feeds and days 5, 7 and 8 for 3 feeds).

19) Assay fusion.

20) When positive wells are approximately 50% confluent, move entire contents to a 24 well containing 2 mLs of fusion media. Note: 1×HT (50×, ICN, cat.# 1680949) should replace 1×HAT in the fusion medium at this point.

See for example, Kearney, J. F., et al., J. Immunol. 123:1548-1550 and Lane, R. D. (1985), J. Immunol. Methods: 81:223-228.

The fusion mixture was distributed into 35 96-well flat-bottomed plates and fed three times with a 70% media replacement after 4, 6 and 7 days. This fusion was called 279.

### Screening of the Fusion

Fusion 279 was screened with all three assay formats detailed above. The ELISA assay on plate adsorbed PROK2 and the ORIGEN solution phase capture assay were performed on day 8 following fusion. The ELISA assay was performed as described earlier except 1) coating of the assay plates with PROK2, addition of undiluted culture supernatant from fusion plates, addition of HRP conjugated goat anti-mouse IgG, Fc specific antisera, addition of TMB and addition of TMB stop solution were all done with 50 uL volumes per well, 2) instead of diluted antisera in the assay plates, undiluted supernatant from each of the wells on the fusion plates was replica plated onto the assay plates and 3) plates were blocked once with PBS-Tween+1% BSA instead of Superblock for 1 hour at RT. The ORIGEN assay was as described earlier except that undiluted supernatant from each of the wells on the fusion plates was replica plated onto the assay plates. After removal of supernatant from the fusion plates for the above two assays, an equivalent amount of fresh media was added back. The following day the PROK2 neutralization assay on Rat2 KZ108 GPR73a cells was performed, again with undiluted supernatant as opposed to dilutions of antisera.

Results of these assays indicated that there were slightly over 150 master well supernatants that either yielded an OD in the ELISA of >1.1 (approximately 20 fold over background) or a relative unit value >5 fold over background in the ORIGEN assay or both and were referred to as positive master wells. For most of these supernatants, both minimal criteria were met and often well exceeded. Of these positive master wells, supernatants from 20 were shown to inhibit PROK2 activity on the Rat2 KZ108 GPR73a cells by 75% or more and all were associated with significantly positive results in both the ELISA and ORIGEN assays. Of the remaining ELISA/ORIGEN positive wells, about 30 demonstrated intermediate levels of inhibition (50-75%) and the rest showed a continuum of inhibition from the 50% level down to no inhibition at all with a number of these demonstrating little or no inhibition.

Hybridoma cells growing in the positive master wells were expanded into culture in 24 well plates. When the density of the 24 well cultures was approximately 4-6×105 cells/mL, the supernatant (approximately 1.5 mL) was individually collected and stored for each well and the cells from each well cryopreserved.

### Selection and Cloning of Master Wells to Isolate Hybridomas Producing Potent Anti-PROK2 Neutralizing MAbs

Each of the new 24 well supernatants was reanalyzed for PROK2 reactive antibody using the plate bound PROK2 ELISA and ORIGEN solution phase capture assays and more importantly for their ability to inhibit PROK2 in the Rat2 KZ108 GPR73a cell-based neutralization assay. Results of these analyses indicated that 16 master well supernatants retained the capacity to inhibit PROK2 activity in the neutralization assay by 75% or more. With the exception of one supernatant, these strong neutralizing supernatants demonstrated excellent binding to plate bound PROK2 and all showed significant binding in the ORIGEN solution phase capture assay (10-45 fold over background).

Cells in the 15 strongest neutralizing master wells (as indicated in this secondary analysis of master well supernatants) were cloned in order to isolate a cloned hybridoma producing the neutralizing mAb of interest. The master wells chosen included 279.39, 279.61, 279.62, 279.69, 279.96, 279.111, 279.121, 279.124, 279.126, 279.133, 279.145, 279.152, 279.154, 279.156 and 279.157.

Cells were cloned in 96 well microtiter cell culture plates using a standard low-density dilution (less than 1 cell per well) approach and monoclonality was assessed by microscopic examination of wells for a single foci of growth prior to assay. Cloning media consisted of fusion media lacking the HAT component (IMDM, 10% FC1 serum, 2 mM L-glutamine, 1× penicillin/streptomycin, 10% hybridoma cloning factor (Roche Applied Science). To address the possibility that no relevant clones might be obtained in the initial attempt to clone the appropriate hybridoma, at least one additional 96-well plate was seeded at 10 cells/well in order to hopefully generate a culture “enriched” for the appropriate hybridoma cells that could serve as the source for a second attempt at formal cloning. In those cases where a second attempt was made from such an “enriched” well, a backup plate seeded at 10 cells/well was again included.

The following cloning protocol was used: Cloning/Minicloning of Hybridoma Cells

Materials:

1) Culture medium (if cells are growing in fusion medium)
Iscove's modified Dulbecco's medium (IMDM—cat.# 12440-053, GIBCO/Invitrogen Corp.)
10% (v/v) fetal clone I serum (cat.# SH30080, HyClone Laboratories)
1× (2 mM) L-glutamine (cat.# 25030-081, GIBCO/Invitrogen Corp.)
1× (100 U/mL:100 ug/mL) penicillin G sodium: streptomycin sulfate (cat.# 15140-122, GIBCO/Invitrogen Corp.)
1×HT (GIBCO/Invitrogen Corp., cat.# 11067-030)
10% (v/v) hybridoma cloning factor (BM Condimed H1, Roche Diagnostics, cat. # 1088947)

Pre-mix first four components then add the latter two at the indicated concentrations.

After all components have been combined, filter the media through a 0.2 um sterile filter unit and place in a 37° C. water bath.

2) Culture medium (if cell are growing in HSFM)
50% (v/v) Hybridoma—SFM medium (GIBCO/Invitrogen Corp., cat. # 12045-076) supplemented with 1× (2 mM) L-glutamine (cat.# 25030-081, GIBCO/Invitrogen Corp.) & 0.5× (100 U/mL:100 ug/mL) penicillin G sodium: streptomycin sulfate (cat.# 15140-122, GIBCO/Invitrogen Corp.)—(HSFM)
50% (v/v) conditioned medium from a heavy culture (media yellow) of P3-X63-Ag8.653.3.12.11 cells growing in HSFM

3) Sterile 50 mL centrifuge tubes (Falcon, cat. # 352070)

4) Hemocytometer with coverslip

5) Inverted microscope

6) Sterile, flat-bottomed 96-well plates (Costar, cat. #3596)

7) Sterile, flat-bottomed half area 96-well plates (Costar, cat. #3696)

8) 1 mL pipetman and tips

9) 200 ul pipetman and tips

10) Sterile 15 mL centrifuge tube (Falcon, cat. # 352096)

11) Electronic multi-channel pipettor and tips (Thermo Labsystems, cat. # 0002206 060, 1500 uL model)

12) Sterile 50 mL polystyrene reagent reservoir (Costar, cat. # 4870)

Procedure:

1) Mix hybridoma cells well in a 24 well with a 1 mL pipetman (set at 1 mL) and count with the use of a hemocytometer.

2) Calculate the number of uLs of a 1:100 dilution of the cells needed to prepare a 35 mL solution with a total of 175 cells. This volume will be used for the clone plates. Also calculate the number of uLs of the same 1:100 dilution needed to prepare a 30 mL solution with 1200 cells. This volume will be used for a back-up 10 cells/well plate.

3) Fill a 15 mL centrifuge tube with 10 mLs of media (lacking cloning factor and HT or conditioned medium in the case of HSFM). Mix the contents of the 24 well again with a 1 mL pipetman and transfer 100 uL to the 10 mL to effect a 1:100 dilution of the cells.

4) Fill one 50 mL tube with 35 mLs of cloning media and another with 30 mLs of cloning media.

5) Cap the 15 mL tube and mix the contents very well by turning the tube upside down, shaking the tube to remove any fluid left in the bottom and returning the tube to the upright position. Do this about 10 times.

6) Quickly un-cap tube and remove the required volume with a 200 uL pipetman and transfer to the 35 mL tube. Rinse tip well in the media. Using a 1 mL pipetman transfer the required volume to the 30 mL tube. Rinse tip well in the media. Cap both tubes securely.

7) Mix the 35 mL tube by turning end-over-end about 10 times. Pour the contents into a sterile reagent reservoir. Plate 150 uL/well into 2 half-area 96 well plates using an electronic multichannel pipettor.

8) Mix the 30 mL tube as above and pour contents into a sterile reagent reservoir. Plate 250 uL/well into 1 standard area 96 well plate.

9) Place plates into an incubator.

10) Score plates microscopically 2-5 days following plating for a single clone vs. multiple clones vs. questionable number of clones per well.

Six to eight days post-plating, supernatants in all wells were screened by ELISA on plate bound PROK2. With the exception of master wells 279.152 and 279.156, in which no positive clones or positive wells on the 10 cells/well plate(s) were obtained and further efforts to clone appropriate hybridoma cells from these masters was suspended, at least one PROK2 specific clone was isolated on the first attempt or subsequent attempts from “enriched” wells originating from 10 cell/well plates. In these successful cases, cells from at least one and up to six wells for each set in which the supernatant was strongly positive for specific mAb and there appeared to be only a single colony of hybridoma growth, were expanded into 24 well cultures and new supernatant collected. Each of these supernatants was tested 1) via serial 4-fold dilution starting with neat supernatant in the immobilized PROK2 ELISA assay and 2) via serial 2-fold or 4-fold dilution in the cell-based PROK2 neutralization assay, to determine which clones in each set possessed the best specific antibody binding and strongest neutralizing titer. Results of these two assays indicated that the two measurements strongly paralleled each other for each clone supernatant (i.e., that the better binding supernatants possessed more potent neutralizing activity) and showed that measurement of anti-PROK2 mAb by ELISA could be used as a surrogate assay for the detection of neutralizing mAb (i.e., they were now one and the same).

Cloning and Screening of Mouse Anti-Human PROK2 (zven1) Antibodies:

The top 15 pools from fusion 279 (mouse anti-human zven1) were identified using a neutralization assay. Each of these master wells (in sets of five) was thawed and cloned after cells recovered two days later (see Protocol #1). Cells were seeded in 96 well plates at 0.75 cells per well and a 10 cell per well backup plate. These plates were scored microscopically 3 to 5 days later to identify single clones vs. multiple or questionable number of clones per well and assayed at 5 to 7 days post-plating. A direct ELISA was used to identify the clones with the best binding capacity (see Protocol #2). The wells with the highest OD readings were examined for cell health and confluency and the top 6 clones chosen from each master well were grown up to 24 well cultures. If there were no positive clones identified, another round of cloning was performed from a positive multi-clonal well.

As the 24-well cultures from the first round of cloning became confluent, a sample was taken and assayed using both the neutralization assay and a direct titration ELISA. In this assay a sample was titrated out using fourfold serial dilutions to see which clone could maintain the highest OD reading. Using the results from both the neutralization and titration assays, one or two clones from each initial master well were chosen to go forward with. Another neutralization screen was performed that ran all these samples in the same assay and at this point the number of cell lines was narrowed down to four top picks. These were subjected to an additional round of cloning to ensure culture homogeneity and screened using the direct ELISA. After one more titration assay, four final clones were chosen: 279.111.5.2; 279.121.7.4; 279.124.1.4; and 279.126.5.6.5.

These were scaled up for purification, weaned from cloning factor and 25 vials of each were banked for ATCC deposit. Mycoplasma testing performed at ZymoGenetics determined all were free of infection.

Protocol #1: Cloning of Hybridoma Cells

Materials:

1) Culture medium (if cells are growing in fusion medium)
Iscove's modified Dulbecco's medium (IMDM—cat.# 12440-053, GIBCO/Invitrogen Corp.)
10% (v/v) fetal clone I serum (cat.# SH30080, HyClone Laboratories)
1× (2 mM) L-glutamine (cat.# 25030-081, GIBCO/Invitrogen Corp.)
1× (100 U/mL:100 ug/mL) penicillin G sodium: streptomycin sulfate (cat.# 15140-122, GIBCO/Invitrogen Corp.)
1×HT (GIBCO/Invitrogen Corp., cat.# 11067-030)
10% (v/v) hybridoma cloning factor (BM Condimed H1, Roche Diagnostics, cat. # 1088947)

Pre-mix first four components then add the latter two at the indicated concentrations.

After all components have been combined, filter the media through a 0.2 um sterile filter unit and place in a 37° C. water bath.

2) Sterile 50 mL centrifuge tubes (Falcon, cat. # 352070)

3) Hemocytometer with coverslip

4) Inverted microscope

5) Sterile, flat-bottomed 96-well plates (Costar, cat. #3596)

6) Sterile, flat-bottomed half area 96-well plates (Costar, cat. #3696)

7) 1 mL pipetman and tips

8) 200 ul pipetman and tips

9) Sterile 15 mL centrifuge tube (Falcon, cat. # 352096)

10) Electronic multi-channel pipettor and tips (Thermo Labsystems, cat. # 0002206 060, 1500 uL model)

11) Sterile 50 mL polystyrene reagent reservoir (Costar, cat. # 4870)

Procedure:

1) Mix hybridoma cells well in a 24 well with a 1 mL pipetman (set at 1 mL) and count with the use of a hemocytometer.

2) Calculate the number of uLs of a 1:100 dilution of the cells needed to prepare a 35 mL solution with a total of 175 cells. This volume will be used for the clone plates. Also calculate the number of uLs of the same 1:100 dilution needed to prepare a 30 mL solution with 1200 cells. This volume will be used for a back-up 10 cells/well plate.

3) Fill a 15 mL centrifuge tube with 10 mLs of media (lacking cloning factor and HT or conditioned medium in the case of HSFM). Mix the contents of the 24 well again with a 1 mL pipetman and transfer 100 uL to the 10 mL to effect a 1:100 dilution of the cells.

4) Fill one 50 mL tube with 35 mLs of cloning media and another with 30 mLs of cloning media.

5) Cap the 15 mL tube and mix the contents very well by turning the tube upside down, shaking the tube to remove any fluid left in the bottom and returning the tube to the upright position. Do this about 10 times.

6) Quickly un-cap tube and remove the required volume with a 200 uL pipetman and transfer to the 35 mL tube. Rinse tip well in the media. Using a 1 mL pipetman transfer the required volume to the 30 mL tube. Rinse tip well in the media. Cap both tubes securely.

7) Mix the 35 mL tube by turning end-over-end about 10 times. Pour the contents into a sterile reagent reservoir. Plate 150 uL/well into 2 half-area 96 well plates using an electronic multichannel pipettor.

8) Mix the 30 mL tube as above and pour contents into a sterile reagent reservoir. Plate 250 uL/well into 1 standard area 96 well plate.

9) Place plates into an incubator.

10) Score plates microscopically 2-5 days following plating for a single clone vs. multiple clones vs. questionable number of clones per well.

Protocol #2: Direct ELISA zven1 (PROK2)

1. Dilute coating antigen in ELISA A Buffer (0.1 M Sodium Carbonate ph9.6).

PROK2 used at 1.27 mg/mL 1 ug/nl (9.4 μl/12 mL)

2. Plate coating antigen, 100 μl/well in 96/well plate(s).

3. Seal plate(s) and incubate overnight at 4 C.°.

4. Wash plate(s) 2×, 300 μl/well, in ELISA C Buffer using plate washer.

5. Block plate(s) with 1% BSA in ELISA C Buffer (ELISA B), 200 μl/well. Incubate 1 hr at RT.

Flick plate(s) to empty.

6. Load CM samples, 50 μL/well, incubate for 1 hour at RT.

7. Wash plate(s) 2×, 300 μl/well, in ELISA C Buffer using plate washer.

8. Dilute 2nd antibody in ELISA B Buffer. Plate 2′ Ab, 100 μl/well. Incubate 1 hour at RT.

HRP Goat anti-Mouse IgG Fc Specific (Jackson 115-035-071, use Concentration: 1:5000)

9. Wash plate(s) 5×, 250 μl/well, in ELISA C Buffer using plate washer.

10. Plate TMB development solution, 100 μl/well. Incubate at room temperature for 5 minutes.

11. Stop color development by plating Stop Solution, 1 00 μl/well.

12. Read plates, OD at 450 nm, within 15 minutes of Stop

Subcloning of the four selected first round clones indicated that a high majority of the subclones derived from 279.111.5 (98.6%) 279.121.7 (100%), 279.124.1 (100%) and 279.126.5.6 (100%) produced antibody reactive with PROK2 and indicated that further subcloning efforts to isolate final clonal hybridomas were not necessary. Cells from 6 wells in each final subclone set for which the supernatant was strongly positive for specific mAb and there appeared to be only a single colony of hybridoma growth were expanded into 24 well cultures. Each of the hybridoma clones was then adapted to growth in media lacking hybridoma cloning factor (IMDM, 10% FC 1 serum, 2 mM L-glutamine, 1× penicillin/streptomycin) by splitting cells into the latter media when cell density was appropriate. Following adaptation, supernatant was collected from the subclones in each set and titered by ELISA on plate bound PROK2. Based on titer with respect to cell density at the time of supernatant collection, a “best” final clone was chosen leading to the selection of the following group of final clones: 279.1111.5.2; 279.121.7.4; 279.124.1.4; and 279.126.5.6.5.

Hybridomas expressing the neutralizing monoclonal antibodies to human PROK2 described above were deposited with the American Type Tissue Culture Collection (ATCC; Manassas Va.) patent depository as original deposits under the Budapest Treaty and were given the following ATCC Accession No.s: clone 279.111.5.2 (ATCC Patent Deposit Designation PTA-6856); clone 279.121.7.4 (ATCC Patent Deposit Designation PTA-6859); clone 279.124.1.4 (ATCC Patent Deposit Designation PTA-6857); and clone 279.126.5.6.5 (ATCC Patent Deposit Designation PTA-6858).

The mouse IgG isotype of the mAb produced by each of these hybridomas was determined using the Mouse Monoclonal Antibody IsoStrip test (Roche Applied Science). All of the mAbs were found to belong to the IgG1 subclass except for 279.124.1.4 which was shown to belong to the IgG₂ₐ subclass. All possessed a kappa light chain.

### Example 31

### Serum Screening of Monoclonal Antibodies

### A. Measured by Luciferase Assay

Serum Screening of Mice

Antibody Inhibition of the Binding and Stimulatory Activity of PROK2 to Rat2 KZ108 GPR73a Cells in Luciferase Assay

Rat2 (rat, fibroblast) cells were stably transfected with a SRE luciferase construct and GPCR73a.

Cells were removed with trypsin, centrifuged at 1300 RPM, room temp, for five minutes.

Resuspend cells in plating media (DMEM, 1% FBS, 1 mM sodium pyruvate, 2 mM L-glutamine, 25 mM Hepes, and counted on a hemacytometer.

Cells were plated on 96 well, flat bottomed, white polystyrene plates (Corning/Costar 3917) at a density of 3,000 cells per well in a volume of 100 ul. Plates were incubated overnight at 370, 5% CO2.

Experiment 1

Assay of Mouse Bleed Three Samples

PROK2 protein was diluted in assay media (DMEM, 0.5% BSA, 1 mM sodium pyruvate, 2 mM L-Glutamine, 25 mM Hepes) to 50 ng/ml. Mouse serum was diluted in assay media at 1:250, 1:500, and 1:1000. Equal volumes of PROK2 and either mouse serum or assay media only were incubated at 370 C for 30 minutes. Final concentration of PROK2 was 25 ng/ml and mouse serum was 1:500, 1:1000, and 1:2000. Previous experiments demonstrated that this is a sub maximal concentration of PROK2 and these mouse serum dilutions have minimal effect on the assay. A dose response of PROK2 from 1000-1 ng/ml with ½ log dilutions was also prepared.

Plates were removed from incubator, media was dumped, and plates were blotted on paper towels to remove excess plating media. Samples were added to wells in duplicate containing PROK2/mouse serum or PROK2/media in 100 ul per well. Control wells contained PROK2 only. An additional plate was prepared with a dose response of PROK2. Plates were incubated at 370 and 5% CO2 for four hours. Media was dumped, plates were blotted on paper towels, and 25 ul of 1× Promega lysis buffer was added to each well. Plates were cooled to room temperature for at least 20 minutes and then read on a luminometer using a three second integration interval. Mouse sample 387 showed inhibition of PROK2 at 1:500 and 1:1000 dilutions.

Experiment 2

Assay of Fusion Samples

Cell plating and assay were the same as previous experiment with the following exceptions.

Monoclonal supernatants in fusion media were received in a total of 36 96 well Costar/Corning V bottom plates (3357) with 130 ul per well. Twenty ul of PROK2 was added to each well to give a final assay concentration of 10 ng/ml. Previous experiments showed that this is a sub maximal concentration in fusion media. Control wells on each plate were 1) PROK2 in fusion media and 2) PROK2 with mouse 387 bleed at 1:500 final concentration in fusion media. All 36 plates were incubated at 370 C for one hour.

Samples were added to plates containing cells and assayed as above.

Experiment 3

Assay of Selected Wells from Fusion

Assay is the same as experiment 2 with the following exceptions. These were from 24 well plates and 157 samples were assayed. Aliquots of each sample were received in two 96 well Costar plates. Each sample was assayed with both 10 and 32 ng/ml PROK2. The higher concentration was chosen to give a more stringent test of antibody potency. Results on P165 are percent response of supernatant sample with PROK2 in relation to PROK2 alone.

### B. Binding of Anti-PROK2 Antibodies to Immobilized PROK2

Sera were screened for IgG antibodies that could bind to PROK2 that had previously been adsorbed onto polystyrene ELISA plates. In this assay, wells of 96 well polystyrene ELISA plates were initially coated with 100 uL/well of PROK2 at a concentration of 1 ug/mL in 0.1M Na2CO3, pH 9.6. Plates were incubated overnight at 4° C. after which unbound antigen was aspirated and the plates washed twice with 300 uL/well of PBS-Tween (0.137M NaCl, 0.0027M KCl, 0.0072M Na2HPO4, 0.0015M KH2PO4, 0.05% v/v polysorbate 20, pH 7.2). Wells were blocked with 200 uL/well of SuperBlock (Pierce, Rockford, Ill.) for 5 minutes at room temperature (RT), the SuperBlock flicked off the plate and the block repeated once more after which the plates were washed twice with PBS-Tween. Serum samples were initially diluted 1:100 in PBS-Tween and subsequently serial 10-fold diluted in PBS-Tween to yield dilutions of 1:100, 1:1,000, 1:10,000 and 1:100,000. Samples of each dilution were added in duplicate to the assay plates, 100 uL/well. Plates were incubated for 1 hour at RT after which unbound antibody was aspirated and the plates washed twice with 300 uL/well of PBS-Tween. HRP conjugated goat anti-mouse IgG, Fc specific antisera (Jackson Immunoresearch) was diluted 1:5000 in PBS-Tween+1% BSA and added to wells of the assay plates, 100 uL/well. Following a 1 hour incubation at RT, unbound second step antibody was aspirated from the wells and the plates washed 5 times. 100 uL/well of tetramethyl benzidine (TMB) (BioFX Laboratories, Owings Mills, Md.) was then added to each well and the plates incubated for 5 minutes at RT. Color development was stopped by the addition of 100 uL/well of 450 nm TMB Stop Reagent (BioFX Laboratories, Owings Mills, Md.) and the absorbance values of the wells read on a Molecular Devices Spectra MAX 340 instrument at 450 nm.

### C. Binding of Anti-PROK2 Antibodies to PROK2 in Solution

Sera were screened for IgG antibodies that could bind to PROK2 in solution using an ORIGEN (Igen Corp.) solution phase capture assay. Briefly, PROK2 was first tagged with ruthenium according to manufacturer's instructions. Just before initiation of assay the stock ruthenium-PROK2 was diluted to a concentration of 100 ng/mL in IMDM-10%-Tween 80 [Iscove's Modified Dulbecco's Medium (Invitrogen)+10% FC1 serum (Hyclone Laboratories)+0.1% Tween 80 (Sigma)]. Serum samples were initially diluted 1:100 in IMDM-10%-Tween 80 and subsequently serial 10-fold diluted in same to yield dilutions of 1:100,1:1,000, 1:10,000 and 1:100,000. Samples of each serum dilution were added in duplicate to 96-well microtiter plates, 100 uL/well and were followed by the addition of 25 uL (2.5 ng) ruthenium-PROK2 to each well. Plates were covered and gently vortexed on a plate vortexer for 2 hours at room temperature (RT). Following the 2 hour incubation, sheep anti-mouse IgG conjugated Dynabeads (Dynal Corp.) were diluted to a concentration of 100 ug/mL in IMDM-10%-Tween 80 and added to the assay plates, 50 uL/well. Plates were again covered, gently vortexed for 30 minutes at RT to keep the beads in suspension and then the relative amount of ruthenium-PROK2 attached to the beads (via anti-PROK2 antibodies) was determined on an M384 analyzer (Igen Corp.).

Assay results from analysis of the first two serum samples from the mice indicated that relatively low titers of anti-PROK2 antibodies existed in all animals, regardless of the assay method used to measure titer. There was a significant improvement, however, at the time of the third serum sampling. ELISA on plate bound PROK2 demonstrated that most of the mice sera still showed significant reactivity (approximately half the maximal OD achievable in the assay) at 1:100,000 dilution. ORIGEN assay results indicated binding levels of IgG antibody 15-20 fold over background at dilutions of 1:10,000. Fifty percent or better inhibition of PROK2 in the Rat2 KZ 108 GPR73a cell-based luciferase assay was still apparent at a 1:1000 serum dilution in 3 of 4 mice tested. Based primarily on the neutralization assay results, the two mice with the highest neutralization titer were chosen for generation of anti-PROK2 mAbs with an emphasis on the generation of mAbs that neutralized PROK2 activity.

### Example 32

Neutralization by Anti-PROK2 Monoclonal Antibodies Measured by GROα Inhibition

Method for screening PROK2 neutralizing monoclonal antibodies for inhibitory activity in GROα secretion assay using Wky12-22 cells.

The initial screen to determine the optimal neutralizing PROK2 monoclonals was performed using the PROK2 activity assay with Rat 2 cells KZ108 (SRE reporter construct) transfected with the GPCR73 a receptor. Medias that had inhibitory activity in this first assay were then further assayed for biological activity in the GROα assay using the Wky12-22 cell line that expresses both PROK2 receptors GPCR73a and b. Monoclonals were ranked on their ability to inhibit PROK2 activity in both in vitro assays.

Background:

Our previous studies showed that the rat aortic smooth muscle cells Wky12-22 cells secrete the chemokine CINC-1, also known as GROα, when treated with zven1 and zven2.

In order to determine the optimum concentration of PROK2 to use in the inhibition assay, a dose response curve was generated using in-house e coli produced PROK2 protein, Peprotech purchased PROK2 protein, and PROK1 protein from Peprotech. The resulting EC50's were: PeproTech PROK1=2.94 ng/ml; PeproTech PROK2=0.15 ng/ml; and In-house E. coli produced PROK2 A1197F=0.55 ng/ml

The maximal effect is seen at 10 ng/ml PROK2 and >100 ng./ml PROK1. The EC50 concentrations result in the secretion of GRO at a concentration of approximately 350 ng/ml. A dose at 80% of maximum was chosen, or 1 ng/ml PROK2 and 5 ng/ml PROK1 to screen for inhibitory activity.

Screening of hybridoma cell culture conditioned medias to look for assay interference: Preliminary screening of four samples of CM from hybridomas was conducted to determine if the medias alone interfered with the GROα readout in Wky12-22 cells. Medias were tested to see if they induced GROα release, or if they inhibited PROK2 induced GROα release

Wky12-22 cells were plated in 24 well plates and grown to 90% confluency in 10% FBS/DMEM cell culture media at 37 degrees centigrade and 5% C02.
Hybridoma conditioned medias without antibody were tested at 10, 33 and 100% concentrations. CM was diluted in assay media consisting of 5% FBS/DMEM.
Total volume/well was 0.5 ml. The 24 well plates of Wky12-22 cells were incubated at 37° C., 5% CO2 for six hours. CM was collected, spun in an eppendorf tube and stored at 4° for short term or frozen @-80° for long term storage until samples can be assayed for GROα using a Rat GRO/CINC-1 Elisa Assay Kit from IBL Co., Ltd. Code No. 17162, Lot # OF-403.
Results: At 100% and 33% media, there was a very small increase in background levels of GROα from Wky12-22 cells.

At 1:10, all medias look good. Medias alone did not inhibit GROα release at any concentration. At 100% media, 0.5 ng/ml PROK2 induced GROα release was slightly increased. Since only diluted CM from monoclonals will be used, this should not be an issue. Media does not interfere with assay. See summary in Table 13 below.

TABLE 13
Picograms/ml GROα

| | | CM | CM | CM | CM |
|---|---|---|---|---|---|
| | | 285.179.12 | 285.234.9 | 283.108.2.3 | 285.234.9 |
| Basal Control | 29.33 pg/ml | 100% = 21.68 | 100% = 24.3 | 100% = 23.6 | 100% = 24.7 |
| 0.5 ng/ml PROK2 | 121.02 pg/ml | 100% = 181 | 100% = 248 | 100% = 229 | 100% = 248 |
| Control | | 33% = 111.5 | 33% = 163.2 | 33% = 155.9 | 33% = 163.2 |
| | | 10% = 106.4 | 10% = 109 | 10% = ND* | 10% = 135.8 |

*ND = data lost

Screening of hybridoma cell culture medias containing antibody to look for inhibition of PROK2 induced GROα release: In the same experiment, neutralizing activity was evaluated in antibody containing Hybridoma CM from three cultures. CM was tested at the same concentrations as above (100%, 33%, 10%), in the presence of 0.5 ng/ml PROK 2 Lot A1197F to determine if CM had inhibitory activity. Monoclonal batches tested were: 279.61.1.3, 279.111.1, and 279.111.4.

Assay was run as above, but prior to adding to Wky12-22 cells, CM samples were incubated for 30 minutes with 0.5 ng/ml PROK2. CM containing PROK2 was then added to cells and incubated for six hours. Samples were tested as described above. Results are outlined in Table 14 below.

TABLE 14
Inhibition of PROK2 induced GROα Release with
monoclonal supernatants pg/ml GROα

| | | CM | CM | CM |
|---|---|---|---|---|
| | | 279.111.4 | 279.61.1.3 | 279.111.1 |
| Basal Control | 29.33 pg/ml | | | |
| 0.5 ng/ml | 121.02 pg/ml | 100% = 35.9 | 100% = 130 | 100% = 40.8 |
| PROK2 | | | | |
| Control | | | | |
| | | 33% = 12.6 | 33% = 77.5 | 33% = 15.8 |
| | | 10% = 21 | 10% = 93.9 | 10% = 14 |

Conclusions: Two of the three monoclonal supernatants have inhibitory activity at all concentrations tested: 279.111.4 and 279.111.1. Sample 279.61.1.3 did not inhibit. This same sample performed poorly in the GPCR73a reporter assay.

Samples 279.111.4 was diluted further and run a second time with more monoclonal supernatants (six total). During this second screen where supernatants were diluted from 1:10 to 1:1250, antibody 279.121.9 inhibited GROα release down to a 1:250 dilution.

Neutralization assay optimization: To make the assay more biologically relevant, the procedure was changed so that the Monoclonal supernatants were not “pre-incubated with the PROK2 protein.

Supernatants containing antibody are added to the cell cultures first, then PROK2 ligand is added. This change in protocol did not affect the inhibitory activity of the monoclonal antibodies.

Screening of purified antibodies to determine IC50 values: The four final PROK2 neutralizing monoclonal antibodies were screened for inhibitory activity and their IC50 (50% inhibition values) calculated.

When purified monoclonal antibodies became available, the assay was run as outlined above with the following changes

The PROK2 ligand challenge was increased to 1 ng/ml final or 100 picomolar (80% challenge): 450 μl diluted Monoclonal was added/well of a 24 well plate. 50 μl 10×PROK2 protein (10 ng/ml) was immediately added to same wells. Antibody concentrations went from 10 pg/ml down to 0.00001 μg/ml. Final IC50 values are shown in Table 15, below. Antibody 279.126.5.6.6 had the best activity.

Results:

TABLE 15
IC50 Values of PROK2 Neutralizing Antibodies in GROα Assay

| | Antibody | | | |
|---|---|---|---|---|
| | 279.126.5.6.5 | 279.124.1.4 | 279.111.5.2 | 279.121.7.4 |
| IC50 ng/ml | 2.5 ng/ml | 6.94 ng/ml | 13.64 ng/ml | 19.92 ng/ml |
| Antibody | IgG1 | IgG2a | IgG1 | IgG1 |
| Class | | | | |
| Ranking in | #1 | #2 | #3 | #4 |
| order of | | | | |
| Potency | | | | |

Ability of PROK2 monoclonal antibodies to inhibit both PROK1 and PROK2 induced GROα release from Wky12-22 cells.

Wky12-22 cells are plated in 24 well plates and grown to approximately 95% confluency. Media is decanted and replaced with assay media RPMI+5% FBS containing test reagents.

Four monoclonal antibodies 279.111.5.2, 279.121.7.4, 279.124.1.4 and 279.126.5.6.5 are added to each of 4 wells at a concentration of 1 ug/ml.

Wells were then challenged with either PROK1 or PROK2 at 1 ng/ml or 0.1 ng/ml.

Control wells are run containing assay media only and assay media plus 0.1 or 1.0 ng/ml PROK1 or PROk2.

Plate is incubated in 5% CO₂ at 37° C. for 6 hours. CM is removed, spun in eppendorf tubes and assayed for GROα

See Table 16 for inhibition results:

TABLE 16
Inhibition of PROK1- and PROK2-induced Groα secretion. All values in pg/ml
GROα

| | | Antibodies @ | | | |
|---|---|---|---|---|---|
| | | 1 ug/ml | | | |
| | | 279.111.5.2* | 279.121.7.4 | 279.124.1.4 | 279.126.5.6.5 |
| Basal | 49.5 pg/ml | | | | |
| Control | | | | | |
| 0.1 ng/ml | 84.28 pg/ml | 52.9 | 45.628 | 55.15 | 46.3 |
| PROK2 + | | | | | |
| Control | | | | | |
| 1.0 ng/ml | 100.5 pg/ml | 53.1 | 50.329 | 57.29 | 54.48 |
| PROK2 + | | | | | |
| Control | | | | | |
| 0.1 ng/ml | 67.03 pg/ml | 53.195 | 52.52 | 56.53 | 55.66 |
| PROK1 + | | | | | |
| Control | | | | | |
| 1.0 ng/ml | 81.9 pg/ml | 54.08 | 60.35 | 61.4 | 66.9 |
| PROK1 + | | | | | |
| Control | | | | | |
| Potency | | #1 | #2 | #3 | #4 |

*Most potent

Conclusions: All antibodies inhibited PROK2 induced GRO release induced by 0.1 or 1.0 ng/ml ligand. All antibodies inhibited PROK1 0.1 ng/ml challenge. Only the PROK2 monoclonal 279.111.5.2 inhibited PROK2 at the highest, 10 ng/ml challenge. This data agrees with the ELISA binding data indicating that the antibodies do cross react with PROK1 also.

### Example 33

### Neutralization of Monoclonal Antibodies by Inhibition of Aoritic Ring Outgrowth Assay

Thoracic aortas were isolated from 4-5 month old SD rats were transferred to petri dishes containing HANK's buffered salt solution (Gibco). The aortas are flushed with additional HANK's buffered salt solution to remove blood and adventitial tissue surrounding the aorta carefully removed. Cleaned aortas are transferred to petri dish containing EBM basal media, serum free (Clonetics, San Diego, Calif.). Aortic rings were obtained by slicing, approximately 1 mm sections using a scalpel blade. The ends of the aortas used to hold the aorta in place were not used. The rings were rinsed in fresh EBM basal media and placed individually in a wells of a 24 well plate coated with Matrigel (Becton Dickinson, Bedford, Mass.). The rings were overlayed with an additional 50 μl Matrigel and placed at 37° C. for 30 min. to allow matrix to gel. Treatments diluted in EBM basal serum free media supplemented with 100 units/ml penicillin, 100 μg/ml streptomycin and HEPES buffer were added 1 ml/well. Background control was EBM basal serum free media alone and bFGF (R&D) at 20 ng/ml was used as a positive control. Samples were added in a minimum of quadruplets. Rings were incubated for 5-8 days at 37° C. and analyzed for growth.

Test Group Concentrations:

100 ng/ml+Neutralizing Ab E8410 (#4) 10 ug/ml

10 ng/ml+Neutralizing Ab 10 ug/ml

1 ng/ml+Neutralizing Ab 10 ug/ml

1 ng/ml+Neutralizing Ab 1 ug/ml

0.1 ng/ml+Neutralizing Ab 10 ug/ml

0.1 ng/ml+Neutralizing Ab 1 ug/ml

100 ng/ml PROK2 was run alone a control

Results indicate that PROK2 induces angiongenesis of the aortic rings.

### Example 34

### Characterization of Monocolonal Antibodies

Monoclonal antibodies from four different clonal hybridomas (279.124.1.4, 279.126.5.6.5, 279.121.7.4, 279.111.5.2) demonstrated the ability to neutralize the activity of PROK2 in a cell-based neutralization assay. The functional binding properties of these monoclonal antibodies were additionally characterized using competitive binding (epitope binning) experiments and Western blotting.

Competitive Epitope Binding (epitope binning):

Epitope binning experiments were performed to determine which antibodies are capable of binding to PROK2 simultaneously. Monoclonal antibodies that compete for the same, or a similar, binding site (epitope) on PROK2 are not able to bind PROK2 simultaneously and are functionally grouped into a single family or “epitope bin”. Monoclonal antibodies that do not compete for the same binding site on PROK2 are able to bind PROK2 simultaneously and are grouped into separate families or “epitope bins”. Experiments were performed using a Biacore 1000™ instrument. Biacore is only one of a variety of assay formats that are routinely used epitope bin panels of monoclonal antibodies. Many references (e.g. The Epitope Mapping Protocols, Methods in Molecular Biology, Volume 6,6 Glenn E. Morris ed.) describe alternative methods that can be used (by those skilled in the art) to “bin” the monoclonal antibodies, and would be expected to provide comparable data regarding the binding characteristics of the monoclonal antibodies to PROK2. Epitope binning experiments are performed with soluble, native antigen.

Materials and Methods:

Epitope binning studies were performed on a Biacore 1000™ system (Biacore, Uppsalla Sweden). Methods were programmed using Method Definition Language (MDL) and run using Biacore Control Software, v 1.2. Polyclonal goat anti-Mouse IgG Fc antibody (Jackson ImmunoResearch Laboratories, West Grove, Pa.) was covalently immobilized to a Biacore CM5 sensor chip and was used to bind (capture) the primary monoclonal antibody of a test series to the chip. Unoccupied Fc binding sites on the chip were then blocked using a polyclonal IgG Fc fragment (Jackson ImmunoResearch Laboratories, West Grove, Pa.). Subsequently, PROK2 (commercially obtained from PeproTech, Rocky Hill, N.J. #100-46, lot # 040429) was injected and allowed to specifically bind to the captured primary monoclonal antibody. The Biacore instrument measures the mass of protein bound to the sensor chip surface, and thus, binding of both the primary antibody and PROK2 antigen were verified for each cycle. Following the binding of the primary antibody and antigen to the chip, a monoclonal antibody of the test series was injected as the secondary antibody, and allowed to bind to the pre-bound antigen. If the secondary monoclonal antibody was capable of binding the PROK2 antigen simultaneously with the primary monoclonal antibody, an increase in mass on the surface of the chip, or binding, was detected. If, however, the secondary monoclonal antibody was not capable of binding the PROK2 antigen simultaneously with the primary monoclonal antibody, no additional mass, or binding, was detected. Each monoclonal antibody tested against itself was used as the negative control to establish the level of the background (no-binding) signal.

A single experiment was completed to test the binding properties of purified monoclonal antibodies from 4 hybridoma clones (279.124.1.4, 279.126.5.6.5, 279.121.7.4, 279.111.5.2). Each antibody was tested as the primary antibody in combination with the entire panel of monoclonal antibodies. All purified monoclonal antibodies were tested at equal concentrations. In between cycles, the goat anti-Mouse IgG Fc capture antibody on the chip was regenerated with 20 mM HCl. Control cycles were run to demonstrate a lack of response of the secondary antibody in the absence of primary antibody or antigen. Data was compiled using BioEvaluation 3.2 RCI software, then loaded into Excel™ for data processing.

Results:

Table 17 summarizes the results of the epitope binning experiment. The signal (RU, response units) reported by the Biacore is directly correlated to the mass on the sensor chip surface. Once the level of background signal (RU) associated with the negative controls was established (a single monoclonal antibody used as both the primary and secondary antibody), the binning results were reported as either positive or negative binding. Positive binding indicates that two different monoclonal antibodies are capable of binding PROK2 simultaneously. Negative binding indicates that two different monoclonal antibodies are not capable of binding PROK2 simultaneously. The differential between positive and negative response values in this experiment was significant, and allowed for an unambiguous assignment of the monoclonal antibodies into two distinct families or epitope bins. The first epitope bin was comprised of monoclonal antibodies from hybridomas 279.124.1.4, 279.126.5.6.5, 279.121.7.4, and the second bin was comprised of the monoclonal antibody from hybridoma 279.111.5.2.

TABLE 17
Epitope binning results for the four neutralizing mouse anti-human
PROK2 monoclonal antibodies:

| | | Secondary | | |
|---|---|---|---|---|
| Primary | 279.121.7.4 | 279.124.1.4 | 279.126.5.6.5 | 279.111.5.2 |
| 279.121.7.4 | − | − | − | + |
| 279.124.1.4 | −* | − | − | + |
| 279.126.5.6.5 | − | − | − | + |
| 279.111.5.2 | + | + | + | − |

*Signal was slightly elevated above background

Western Blotting:

The ability of the neutralizing monoclonal antibodies from 4 hybridoma clones (279.124.1.4, 279.126.5.6.5, 279.121.7.4, 279.111.5.2) to detect non-reduced and reduced human PROK2 from two sources was assessed using a Western blot format. A rabbit polyclonal antibody known to detect PROK2 in a Western blot format was used as a positive control. Monoclonal antibodies from all four hybridoma clones detected non-reduced human PROK. Under these conditions (one antigen concentration and one antibody concentration) no cross reactivity with human PROK1 was detected.

Materials and Methods:

The human PROK2 antigen was obtained from two sources: PROK2 was either produced in E. coli in house or commercially obtained from PeproTech (Rocky Hill, N.J. #100-46, lot # 040429). The human PROK1 antigen was obtained from PeproTech (Rocky Hill, N.J. #100-44, lot # 0403244). The antigen (100 ng/lane) was loaded onto 4-12% NuPAGE Bis-Tris gels (Invitrogen, Carlsbad, Calif.) in either non-reducing or reducing sample buffer (Invitrogen) along with molecular weight standards (SeeBlue; Invitrogen), and electrophoresis was performed in 1×MES running buffer (Invitrogen). Following electrophoresis, protein was transferred from the gel to 0.2 μm nitrocellulose membranes (Invitrogen). The nitrocellulose blots were blocked overnight in 2.5% non-fat dried milk in Western A buffer (ZymoGenetics, 50 mM Tris pH 7.4, 5 mM EDTA, 150 mM NaCl, 0.05% Igepal, 0.25% gelatin) then cut into sections and exposed to each antibody (0.2 μg/mL of each monoclonal or 2 μg/mL of the rabbit polyclonal antibody in Western A buffer). The blots were then probed with a secondary antibody conjugated to horseradish peroxidase; sheep anti-mouse IgG-HRP (Amersham: Piscataway, N.J.) for the monoclonal antibodies and donkey anti-rabbit Ig-HRP (Amersham) for the polyclonal antibodies. Bound antibody was detected using a chemiluminescent reagent (Lumi-Light Plus Reagent: Roche, Mannheim, Germany) and images of the blots were recorded on a Lumi-Imager (Mannheim-Boehringer).

Results:

Monoclonal antibodies from all four hybridoma clones detected non-reduced PROK2, but did not detect reduced PROK2 on Western Blots. Monoclonal antibodies from hybridoma clone 279.111.5.2 detected PROK2 with a visibly weaker signal than monoclonal antibodies from clones 279.124.1.4, 279.126.5.6.5, and 279.121.7.4 suggesting that the binding properties of this monoclonal antibody differs from those produced by the other three hybridomas. The polyclonal control antibody detected both denatured and denatured/reduced human PROK2. None of the antibodies detected the related antigen, human PROK1.

MAbs from clones 279.62 and 279.121 appeared to recognize the same or very similar epitopes and both of these appeared to share some epitope reactivity (overlap or spatial proximity of recognized epitopes) with 279.69, 279.124 and 279.157. MAbs from the 279.111 clones appeared to react with an epitope distinct from the others. Based on these results, first round clones 279.111.5, 279.121.7 and 279.124.1 were subcloned using the cloning procedure described earlier and screened using the immobilized PROK2 ELISA. In addition, a first round clone from 279.126 (279.126.5.6), which was obtained later than the others and did not make it into the aforementioned assays, was included in the subcloning effort since supernatant taken from low cell density cultures of this hybridoma appeared to be as potent in the PROK2 neutralization assay as some of the other most potent mAbs whose supernatants had been obtained from higher cell density cultures.

The epitope binning and Western blot results support the assignment of the neutralizing monoclonal antibodies raised against human PROK2 into two distinct families or epitope bins. The first epitope bin is comprised of monoclonal antibodies from hybridomas 279.124.1.4, 279.126.5.6.5, 279.121.7.4, and the second bin is comprised of the monoclonal antibody from hybridoma 279.111.5.2.

### Example 35

### PROK2 Induces Angiogenesis in Dorsal Airsac Model

PROK2 was administered in a Dorsal Airsac model according to the proceudure as described by Goi, et al., Cancer Research, 64: 1906-1910, 2004. Breifly, transiently transfected SW620 mouse colon carcinoma cells were places in a sterile chamber, which was placed in the air sac of a nude mouse and the protein was allowed to express. After one week the chamber was removed and the local tissue was examined for hemorrhage and vascular branching. The results show that PROK2 induced vascular branching and localized hemorrhaging, showing that PROK2 is angiogenic. The experiment can be performed with stably transfected SW620 cells as well.

Thus, the monoclonal antibodies described herein will be useful to inhibit hemaorrage and vascular branching.

### Example 36

Neutralization of Reporter Assay Activity by PROK2 Monoclonal Antibodies

Luciferase based PROK2 Activity Assay was performed according to the following procedure.

Materials:

Cells: Rat-1 fibroblast cells that have been transfected with the KZ108 (SRE) luciferase construct using G418 selection and then with the GPCR73a receptor using puromycin selection.

Growth Media: DMEM, 10% FBS, 2 mM L-Glutamine, 1 mM NaPyruvate, 500 ug/ml G418, 2 ug/ml puromycin

Cells should not be allowed to become confluent.

Splitting the cells: When they become almost confluent, split the cells 1:5 or 1:10 if you need them within 2-3 days, or 1:20 if you need them 4-5 days later.

Freezing the cells: Trypsinize and spin down confluent cells, bring up them in 90% serum-10% DMSO, aliquot them and freeze them for later use

Plating Media: DMEM, 1% FBS, 2 mM L-Glutamine, 1 mM NaPyruvate

Assay Media: DMEM, 0.5% BSA, 2 mM L-Glutamine, 1 mM NaPyruvate, 25 mM HEPES

Lysis Buffer: Cell Culture Lysis Reagent (5×), PT#E153A, Promega

Assay Substrate: Luciferase Substrate and Buffer from Promega (located in the Promega freezer, stock room).

Negative Control: monoclonal antibody supernatant

PROK2: In-house purified protein.

Cell Preparation: When the cells get confluent, aspirate the media from the flask, add 4 ml of PBS to wash (if using 10 cm plate use 2 ml PBS and 2 ml Trypsin instead).

Aspirate PBS and add 4 ml of Trypsin-EDA to the cells. Incubate at 37° C. for 2 minutes. Check the cells under microscope to observe loose cells.

Add 16 ml of growth media (10% FBS), and spin the cells at 1000-1300 rpm for 5 minutes with high break on at room temperature.

Aspirate the media and resuspend in 1 ml of plating media (1% FBS). Use 1 ml pipette to disperse the cell clumps. Bring the volume to 10 ml with plating media and count the cells on hemocytometer (mix well and pipette 10 ul for counting).

Dilute cells in plating media to 10⁵ cells/ml and add 100 ul/well to Costar3917-96 well white plates (final concentration is going to be 10,000 cells/well If you are short of cells, you can go down on concentration as low as 8,000 cells/well). Also add cells to one column of a clear plate to check cell density the next day.

Incubate these plates at 37° C. overnight.

Reagent Preparation for Testing:

Prepare standard curve dilutions in assay media. First, dilute PROK2 to 1 ug/ml then do ½ log dilutions.

Prepare samples for the assay (you may need to run samples straight or with several dilutions) on a deep-well plate. Keep the samples volumes the same in each well.

Hybridoma Supernatants: Prepare sample dilutions using fusion media. Also, prepare PROK2 in the same media and add onto the samples with a final concentration of 5 ng/ml. (e.g. if the sample volume is 100 ul, then add 25 ul of 25 ng/ml PROK2 to the plate to get 5 ng/ml final PROK2 concentration). Incubate for 30 minutes at 37° C. Then proceed to the next step. Overheads 1-11 represent data generated from the 1^{st} screens of each masterwell (see powerpoint file “entire luciferase assay data”. Overheads 12-22 are data from 2^{nd} screenings.

Purified Monoclonal Antibodies: Prepare sample dilutions using assay media. Also, prepare PROK2 in the same media. Unlike hybridoma supernatants, there is no 30 minutes preincubation period for purified monoclonal antibodies. First, add the antibody dilutions to the cells and then, add PROK2 to them with a final concentration of 30 ng/ml. Then continue with 4 hour incubation. See power point slide #24 for dose response curve with prok2 illustrating 80% activity. Slide #23 is final EC50 plots with linear regressions.

Dump the assay plate and blot on gauze pads. Then add 100 ul of samples, controls and the standards to the appropriate wells (Leave row A and row H empty and use rows B through G in order not to have edge effect).

Controls: diluent alone (no antibody or PROK2)

PROK2 alone (no antibody)

Incubate plates at 37° C. for 4 hours.

Dump the plate and blot on gauze pads. Add 25 ul of 1× Promega lysis buffer to each well. Let the plate sit on the bench for ≧20 minutes to equilibrate at room temperature.

Stock solution is 5× and it is very viscous. Pour 5 ml into a 50 ml Falcon tube and bring the volume to 25 ml with deionized water. Prepare this solution close to the end of 4-hour incubation period.

Take out Luciferase assay substrate and the buffer an hour before the end of 4-hour incubation. Put them into water bath for 10 minutes then let them sit on the bench until you are ready to read the plates (Luciferase substrate must be at room temperature for assay to work properly).

Add 40 ul of Promega E4550 luciferase substrate to the plates. Substrate addition and reading the plate are being done on Berthold instrument as following:

Open LB96VR Control Window. Put dI-H₂O to the water container, put the tubing in and close the lid. Hit wash and say yes to the prompt. Hit “New” on either “A” or “B” section. It will prompt Login window. Login to the machine, and put comments if you need to. Make sure the substrate bottle (which has aluminum foil) is empty. Add the substrate solution to this bottle put the tubing in and close the lid. Select “40 ul injection with 3 second integration” from protocol tab. Select “Robotic” from Run Mode Tab. Select number of plates to be run on the machine. First prime the instrument by hitting the prime button. Once this is done, hit start.

Export the results to MS Excel format. Plot the standard curve using PROK2 dose response read outs. If you run inhibition assay, calculate % inhibition values of the samples and plot the results as % inhibition vs. samples with ascending dilution series.

% Inhibition=(Negative Control Read Out—Sample Read Out)*100

Negative Control Read Out

Results:

We screened 121 hybridoma supernatants using this activity assay. From these samples, four of them with the best titer were chosen and from which monoclonal antibodies were purified. Neutralizing activities of these purified antibodies were tested using the same KZ108 GPR73a Luciferase based activity assay with 30 ng/ml PROK2 challenge. The EC₅₀ values and were determined and the four monoclonal antibodies were ranked as shown in Table 18.

TABLE 18

| | Antibody | | | |
|---|---|---|---|---|
| | 279.126.5.6.5 | 279.124.1.4 | 279.121.7.4 | 279.111.5.2 |
| EC50 ng/ml | 2.65 μg/ml | 3.84 μg/ml | 4.16 μg/ml | 5.70 μg/ml |
| Ranking in | #1 | #2 | #3 | #4 |
| order of | | | | |
| Neutrali- | | | | |
| zation | | | | |
| Potency | | | | |

The purified monoclonal antibody with the clone number of 279.126.5.6.5 appears to be the best neutralizing monoclonal antibody.

### Example 37

### PROK2 and PROK1 Expression Profiling of Cancer and Normal Tissue

PROK2 and PROK1 Expression Profiling of Cancer/Normal tissue pairs using TaqMan RT-PCR:

Tissue preparation: Cancerous and normal tissue sections from colon, esophagus, pancreas, small bowel, small intestine, stomach, endometrium (cancer only), kidney, liver, lung, mammary gland, skin, and testes were collected from the same patients and flash frozen in liquid nitrogen immediately. Note, the majority of samples were from colon, with the other tissues being represented by six or fewer donors. Tissue samples are obtained from CHTN (Cooperative Human Tissue Network). The company sent us the tissue samples that they labeled as cancer or NAT (Normal adjacent tissue). Tissues are flash frozen in liquid nitrogen within 2 hours.

Total RNA was purified from cancer and normal tissues using an acid-phenol purification protocol (Chomczynski and Sacchi, Analytical Biochemistry, 162:156-9, 1987). The RNAs were then DNAsed using DNA-free reagents (Ambion, Inc, Austin, Tex.) according to the manufacturer's instructions. The RNAs were quantitated by three independent measurements on a spectrophometer, and the quality of the RNA was assessed by running an aliquot on an Agilent Bioanalyzer. Presence of contaminating genomic DNA was assessed by a PCR assay on an aliquot of the RNA with zc41011 (5′CTCTCCATCCTTATCTTTCATCAAC3′; SEQ ID NO: 30) and zc41012 (5′CTCTCTGCTGGCTAAACAAAACAC3′; SEQ ID NO: 31), primers that amplify a single site of intergenic genomic DNA. The PCR conditions for the contaminating genomic DNA assay were as follows: 2.5 ul 10× buffer and 0.5 ul Advantage 2 cDNA polymerase mix (BD Biosciences Clontech, Palo Alto, Calif.), 2 ul 2.5 mM dNTP mix (Applied Biosystems, Foster City, Calif.), 2.5 ul 10× Rediload (Invitrogen, Carlsbad, Calif.), and 0.5 ul 20 uM zc41011 and zc41012, in a final volume of 25 ul. Cycling parameters were 94 oC 20″, 40 cycles of 94 oC 20″ 60 oC 1′20″ and one cycle of 72 oC 7′. 10 ul of each reaction was subjected to agarose gel electrophoresis and gels were examined for presence of a PCR product from contaminating genomic DNA. If contaminating genomic DNA was observed, the total RNA was DNAsed again, then retested as described above.

RNA extraction: Frozen tissue sections were crushed and resuspended in lysis buffer (included in Qiagen kit) containing □ME. RNA isolation performed using RNeasy RNA isolation kit (Qiagen), following manufacturer's instructions.

RNA clean-up: Because DNA and RNA have very similar chemical properties, it is almost impossible to isolate RNA without some DNA contamination. DNase treatment is performed using Superase-In DNase-free kit (Ambion, following manufacturer's instructions.

Quality and Quantity Check: Quality of the RNA samples are determined on HP-Bioanalyzer, using eukaryotic total RNA nano protocol from the assays menu. For quantity determination, absorbances at 260 nm are read and using the following formula, concentrations are determined:

Quantity of sample X=OD₂₆₀*DF*40 ng/μL

DF=1/dilution

1 unit of 260 reading=40 ng/μl

Expression Analysis:

PROK2 and PROK1 standard curve preparation: Synthetic RNA templates were prepared by HDST. Template dilutions were set to 10⁸, 10⁷, 10⁶, 10⁵ and 10⁴ and used to calculate standard curve. Normal human testes RNA were prepared at different concentrations (200, 100, 50, 25 and 10 ng/μl) to serve a standard curve for housekeeping gene.

Primer and probe preparation: primer and the probe sets were designed for both PROK2 and PROK1. As an endogenous control, human glucuronidase (GUS) expression is tested. Primer and probe set for GUS are available in-house.

Sample preparation: RNA samples were thawed in ice and then diluted to 50 ng/□l in RNase-free water (Invitrogen, Cat# 750023). Diluted RNA samples were kept in ice until use.

Master Mix preparation: TaqMan EZ RT-PCR Core reagents (Applied Biosystems, Cat# N808-0236) is used to prepare multiplex master mixes for both PROK2 and PROK1. See Table 19 below).

TABLE 19
Multiplex Master Mix Recipe (per sample)

| Component | Volume/sample (uL) | Final Concentration | |
|---|---|---|---|
| Rnase-free water | 9.45 | — | |
| 5x TaqMan EZ Buffer | 5 | 1x | |
| 25 mM Manganese acetate | 3 | 3 | mM |
| 10 mM deoxyATP | 0.75 | 300 | μM |
| 10 mM deoxyCTP | 0.75 | 300 | μM |
| 10 mM deoxyGTP | 0.75 | 300 | μM |
| 0 mM deoxyUTP | 0.75 | 600 | μM |
| Forward Primer: PROK2 (or | 1 | 800 | nM |
| PROK1) 20 pMoles/λ | | | |
| Reverse Primer: PROK2 (or | 1 | 800 | nM |
| PROK1) 20 pMoles/λ | | | |
| FAM/TAMRA Probe: | 0.025 | 100 | nM |
| PROK2 (or PROK1) | | | |
| 100 pMoles/λ | | | |
| Forward Primer: huGUS | 0.125 | 100 | nM |
| 20 pMoles/λ | | | |
| Reverse Primer: huGUS | 0.125 | 100 | nM |
| 20 pMoles/λ | | | |
| VIC Probe: huGUS | 0.025 | 100 | nM |
| 100 pmoles/λ | | | |
| AmpErase UNG | 0.25 | 0.01 | U/μL |
| rTth DNA Polymerase | 1 | 0.1 | U/μL |

| | | |
|---|---|---|
| Total | 24 | — |

To assay samples in triplicate, 3.5 μl of each RNA sample and controls are aliquoted into optical tube strips (Applied Biosystems, Cat# 4316567). For positive control, human testes standard curve dilutions are used. For negative control, 3.5 μl of RNase-free water (no template control) is used. Then 84 μl of PCR multiplex master mix added and mixed well by pipetting.

MicroAmp Optical 96-well plate (Applied Biosystems, Cat# N801-0560) is placed on ice and 25 μl of RNA/master mix is added in triplicates to the appropriate wells. Then optical adhesive cover (Applied Biosystems, Cat# 4311971) is applied to the plate surface with the applicator and then the plate is spun for two minutes at 300 rpm in the Qiagen Sigma 4-15 centrifuge. A compression pad (Applied Biosystems, Cat# 4312639) is put on top of the plate.

Running the ABI 7000 instrument and Data Analysis: Sequence detector is launched and it is set to real time PCR. Fluorochromes are set to FAM (for PROK2 or for PROK1) and to VIC (for GUS). Plate template is set indicating where standards and where the unknowns are. Thermocycling conditions are: Hold-1 at 50° C. for 2 minutes, Hold-2 at 60° C. for 30 minutes, Hold-3 at 95° C. for 5 min, and 40 cycles at 94° C. for 20 seconds, and 60° C. for 1 minute. After the experiment is over, data analysis is performed per the manufacturer user bulletin #2.

Expression for each sample is reported as a Ct value. The Ct value is the point at which the fluorochrome level or RT-PCR product (a direct reflection of RNA abundance) is amplified to a level, which exceeds the threshold or background level. The lower the Ct value, the higher the expression level, since RT-PCR of a highly expressing sample results in a greater accumulation of fluorochrome/product which crosses the threshold sooner. A Ct value of 40 means that there is no product measured and should result in a mean expression value of zero. For each sample is being tested, Ct values for gene of interest (PROK2 or PROK1) and housekeeping gene (GUS) are determined. The expression is represented as percent ratio to GUS, which is calculated by the following formula:

Percent Ratio to GUS=(2^{-Ct of GOI}/2^{-Ct of HKG})*100

GOI=Gene of Interest (PROK2 or PROK1)

HKG=House Keeping Gene (GUS)

Results: Expression analysis of these samples indicated that in eleven of nineteen patient samples tested, there is a trend toward increased expression of PROK2 in cancer tissue versus normal tissue from the same donor in colon cancer patients.

### Example 38

### Human PROK2 ELISA

NUNC Maxisorb 96-well plates were coated overnight at 4° C. with mouse monoclonal Ab raised against human Prok2 (capture Ab). Coating was done in ELISA A buffer: 0.1M Na₂CO₃, pH adjusted with HCl to 9.6.

After 3 washes with ELISA C (PBS1× with Tween-20 0.05% v/v) samples and standards were added. Standards and sample dilutions were made in ELISA B (ELISA C+2% BSA).

The plates were then placed at 37° C. for 1 h.

After this incubation, plates were washed three times with ELISA C, and a biotinylated mouse monoclonal Ab raised against human PROK2 (detection Ab) was added. Plates were returned at 37° C. for 1 h.

At the end of this period, the plates were again washed three times with ELISA C. SA-HRP (streptavidin-horseradish peroxidase) reagent in ELISA B was added to the plates, which were then placed for 1 h at 37 oC.

The plates were then washed three times with ELISA C and TMB, an HRP substrate, was added. Color was let to develop for 10, before a stop solution was added.

The plate was then read by an ELISA plate reader at 450 nm with a 540 nm subtraction.

Using this method with antibody from clone number 279.124.1.4 as the capture antibody and the antibody from clone number 279.111.5.2 as the detection antibody the OD values for concentrations of PROK2 were as follows: 0 ng/ml=0.017 OD; 0.3 ng/ml=0.049 OD; 1 ng/ml=0.094 OD; 3 ng/ml=0.222 OD; 10 ng/ml=0.788 OD; 30 ng/ml=1.155 OD; 100 ng/ml=1.448 OD; and 300 ng/ml=1.331 OD.

It was shown that the combination of these two monoclonal Abs is useful in detecting human Prok2 in an ELISA format in a dose-dependent fashion.

### Example 39

### PROK2 Effects on Serum Cytokines and Vascular Leak

### A. Analysis of PROK2 on Serum Cytokines

IL-2 therapy is effective in the treatment of certain cancers. However, the use of IL-2 as a therapeutic agent has been limited by its toxic effects, namely vascular leak syndrome (VLS). IL-2 induced VLS is characterized by infiltration of lymphocytes, monocytes and neutrophils into the lung causing endothelial damage in the lung eventually leading to vascular leak (reviewed in Lentsch A B et al, Cancer Immunol. Immunother., 47:243, 1999). VLS in mice can be induced with administration of repeated high doses of IL-2 and measuring vascular leak by Evan's Blue uptake by the lung. Other parameters that have been shown to be characteristic of VLS in mice include increased serum levels of TNFα and IFNγ (Anderson J A et al, J. Clin. Invest. 97:1952, 1996) as well as increased numbers of activated T, NK and monocytes in various organs. Blocking of TNFα with a soluble TNFR-Fc molecule inhibited lung infiltration by lymphocytes and therefore lung injury (Dubinett S M et al, Cell. Immunol. 157:170, 1994). The aim is to compare the ability of IL-2 and PROK2 to induce VLS in mice and to measure the different parameters indicative of VLS (Evan's Blue uptake, serum cytokine analysis, spleen cellular phenotype).

Mice (female, C57B16, 11 week old; Charles River Labs, Kingston, N.Y.) are divided into five groups. All groups contained 10 mice per group. Groups are as follows: Group I or Vehicle group receives Phosphate Buffered Saline (PBS); Group II and III receives PROK2, and Group III receives a PROK2 monoclonal antibody. The study consists of 4 days, body weight is measured daily and animals receive 7 intraperitoneal injection of test substance over the 4-day period. Animals receive two daily injections on day 1-3 and on the fourth day received a single morning injection. Two hours post final injection animals receive a tail vein injection of 1% Evan's blue (0.2 ml). Two hours post Evan's blue injection mice are anesthetized with Isoflurane and blood is drawn is serum cytokine analysis. Following blood draw animals are transcardial perfused with heparinized saline (25 U hep/ml saline). Following perfusion spleen is removed and weighed, liver and lung are removed and placed into 10 mls of formamide for 24 hr incubation at room temperature. Following 24 hr incubation vascular leakage is quantitated by Evan's blue extravasation via measurement of the absorbance of the supernatant at 650 nm using a spectrophotometer.

Mice are bled and serum separated using a standard serum separator tube. 25 μl of sera from each animal is used in a Becton Dickenson (BD) Cytokine Bead Array (Mouse Th1/Th2 CBA Kit) assay. The assay is done as per the manufacturer's protocol. Briefly, 25 μl of serum is incubated with 25 μl bead mix (IL-2, IL-4, IL-5, TNFα and IFNγ) and 25 μl PE-detection reagent for two hours at room temperature in the dark. A set of cytokine standards at dilutions ranging from 0-5000 pg/ml is also set up with beads as per the manufacturer's instructions. The incubated beads are washed once in wash buffer and data acquired using a BD FACScan as per instructions outlined in the Kit. The data is analyzed using the BD Cytometric Bead Array Software (BD Biosciences, San Diego, Calif.).

### B. Analysis of PROK2 on Vascular Leak—Immunophenotyping of Splenic Cells

IL-2 induced vascular leak syndrome (VLS) involves organ damage that occurs at the level of postcapillary endothelium. However, this damage occurs secondary to two distinct pathological processes: the development of VLS, and transendothelial migration of lymphocytes. Acute organ injury is mediated by infiltrating neutrophils while chronic organ injury is mediated by infiltration monocytes and lymphocytes (reviewed in Lentsch A B et al, supra.). In mice, depletion of cells with surface phenotypes characteristic of LAK or NK cells ameliorates organ damage (Anderson T D et al, Lab. Invest. 59:598, 1988; Gately, M K et al. J. Immunol., 141:189, 1988). Increased numbers of NK cells and monocytes is therefore a marker for IL-2 mediated cellular effects of VLS. In addition, IL-2 directly upregulates the expression of adhesion molecules (i.e. LFA-1, VLA-4 and ICAM-1) on lymphocytes and monocytes (Anderson J A et al, supra.). This increase is thought to enable cells to bind activated endothelial cells and help in transmigration of cells to the tissue. Increased expression of these molecules is considered another marker of IL-2 induced cellular activation during VLS. The aim of this study is to study splenic cells from IL-2 and PROK2 treated mice under a VLS protocol and compare the effects of the two cytokines to mediate cellular effects associated with VLS.

Groups of age and sex matched C57BL/6 mice treated and described above (Example 17A) are analyzed. On d4, mice are sacrificed and phenotype of splenic cell populations studied by standard flow cytometry. Splenic weight and cellularity are measure in IL-2 treated mice compared to PBS treated mice.

Spleens are isolated from mice from the various groups. Red blood cells are lysed by incubating cells for 4 minutes in ACK lysis buffer (0.15M NH4Cl, 1 mM KHCO3, 0.1 mM EDTA) followed by neutralization in RPMI-10 media (RPMI with 10% FBS). The expression of cell surface markers is analyzed by standard three color flow cytometry. All antibodies are obtained from BD Pharmingen (San Diego, Calif.). Fluorescin-isothiocyanate (FITC) conjugated CD11a (LFA-1), CD49d (VLA-4, a chain), Gr-I FITC, phycoerythrin (PE) conjugated CD4, NK1.1, CD11b and CyC-conjugated CD8, CD3 and B220 are used to stain cells. 1-3×106 cells are used for individual stains. Non-specific binding is blocked by incubating cells in blocking buffer (PBS, 10% FBS, 20 ug/ml 2.4G2). After blocking, cells are incubated with primary antibodies for 20 minutes. Unless specified otherwise, all mAbs are used at 1 ug/stain in a volume of 100 ul. Cells are washed once in 1×PBS and resuspended in PBS before being acquired using the FACScan or FACSCalibur instruments (BD Biosciences, San Diego, Calif.). Data is analyzed using the Cellquest Software (BD Biosciences).

In addition, additional endpoints are measured between groups. The following endpoints are compared: Body weight, spleen weight, vascular leakage in lung and liver, and serum cytokines. Vascular leakage is also measured in both lung and liver.

### Example 40

### Association of PROK Receptors with Cancer by Immunohistochemistry

A) Cell and Tissue Preparations

Positive control cells consisted of 293FT cells transiently transfected with sequences of gpr73a or gpr73b. Negative controls cells consisted of untransfected 293FT cells.

Control cells were as follows: C06-2593: 293FT cells transiently transfected human gpr73a; C06-2594: 293FT cells transiently transfected human gpr73b; and C06-2595: 293FT cells (untransfected).

Other tissues examined include: gastrointestinal tissue from Genomics Collaborative Inc. (Cambridge, Mass.); gastrointestinal tissue from NDRI (New York, N.Y.); gastrointestinal tissue from ProteoGenex, Inc. (Culver City, Calif.); and gastrointestinal tissue, breast, and lung from Asterand, plc. (Detroit, Mich.).

The cells and tissues described above were fixed overnight in 10% NBF and embedded in paraffin using standard techniques.

B) Immunohistochemistry

5 μM sections were baked at 61° C. for 15 min for tissue adhesion. Slides were subsequently dewaxed in 3×5′ in xylene and rehydrated through graded alcohols as follows: 2×2′ in 100% EtOH, 1×2′ in X95% EtOH, 1×2′ in 70% EtOH. Slides were rinsed in dH20, then either heat induced epitope retrieval (HIER) was performed for 20 minutes under steam followed by 20 minutes cooling to RT in 10 mM Tris, 1 mM EDTA, pH 9.0 or enzyme induced epitope retrieval (EIER) was performed by digesting tissue sections with proteinase K (catalog# 03115844001, Roche)

Slides were loaded onto a DakoCytomation Autostainer. Slides were rinsed with TBS/Tween buffer (TBST), prepared as recommend by manufacturer. Endogenous biotin was blocked with a 10-minute incubation in avidin solution, washed in TBST followed by a 10-minute incubation in biotin solution. Slides were washed in TBST. A protein block (1% BSA ELISA Plate Block solution, ZGI reagent) (EPB) was applied for 30 minutes and blown off slides. Primary antibodies were diluted in EPB and applied for 60 minutes at RT.

Tissues washed twice in TBST, and then incubated 45 minutes in biotinylated Goat anti-Rabbit Ab, 750 ng/ml in EPB (catalog #BA-1000, Vector Labs). Slides washed twice in TBST. Vectastain Elite ABC-HRP Reagent (catalog# PK-7100, Vector Labs) was incubated for 45 minutes. Slides washed twice in TBST. Signals were developed with DAB+ (catalog# K-3468, DakoCytomation) for 10 minutes at room temperature. Tissue slides were then counterstained in hematoxylin (catalog# H-3401 Vector Labs), dehydrated and coverslipped with mounting medium (catalog# 4111, Richard Allen Scientific).

C) Antibody Information:

Rabbit anti-Prokineticin Receptor 1 (GPR73A), affinity purified, Novus Biologicals NLS 3152 (referred to as GPR73× since it recognizes both gpr73a and gpr73b)

Rabbit anti-Prokineticin Receptor 1 (GPR73A), affinity purified, MBL LS-A6684

D) Summary of Major Findings:

In colon cancer, there is an increased level of expression in more than 50% of the adenocarcinoma cells with both antibodies. This suggests the expression level of either GPR73a or both GPR73a and b receptor(s) are increase in colon cancer.

In breast cancer, there is no significant change in the expression levels in the epithelium of normal or cancerous tissues. Approximately 100% of both normal and cancer cells stained positive for the GPR73x (Novus) antibody. This data suggests that either GPR73a or both GPR73a and b receptor(s) are present in the epithelium of breasts. Comparing the relative intensities of the 2 antibodies in cancer and normal control samples, the data also suggests that the GPR73b is more prevalent in the epithelium of normal cells compare to the epithelium cancers due to the fact that there is a trend of slightly higher staining in the cancerous epithelium with GPR73a (MBL) antibody. Another novel finding is that most of endothelial cells in both normal and cancer samples are positive. The endothelium signal was not observed in either gastrointestinal or lung samples.

In lung cancer, both antibodies showed positive staining in greater than 70% of the cancer cells. There is a medium level of normal bronchial epithelium signal with the GPR73x (Novus) antibody, however, no detectable staining was observed in the bronchial epithelium with the GPR73a (MBL) antibody. This may suggest that possibly bronchial epithelium expresses predominantly GPR73b rather a GPR73a.

All above staining pattern comparison between the antibodies are based on the assumption that these two antibodies are specific and have similar affinity and properties towards GPR73a protein.

### Example 41

### Upregulation of PROK2 RNA in IL-10-Stimulated Human Macrophage Cells

Materials and Methods: Monocytes were collected from PBMNC by negative selection from a donor. Briefly, whole peripheral blood (PB) was diluted 1:2 in PBS, underplayed with Ficol, then centrifuged at 200 rpm for 20 minutes at RT. The monocyte-containing interface was then collected and washed several times in PBS. Monocytes were isolated by negative selection using the Dynal kit. Monocytic cells were washed 1×, then resuspended in assay media (RPMI-1640, 10% FBS, 2-ME, L-glutamine and sodium pyruvate). Cells at 3×10⁵/ml were then cultured in media containing 50 ng/ml hCSF-1 (R&D Systems, lot#CC105041) +/−50 ng/ml hIL-10 (R&D Systems, #55) using 6-well (3 mls/well) low adhesion plates (Costar, #3471). Cells were cultured at 5% CO₂, 37° C. for 7 days. On day 6, 100 ng/ml E. coli-derived LPS (Sigma, L-4391, 78H4122) was added to several wells (CSF-1 alone only). RNA from macrophage was prepared and probed for PROK 1 and PROK2 transcripts.

Results: While there was only a weak signal for PROK1 RNA in macrophage derived in CSF-1 alone, there was a significant upregulation for PROK2 message in macrophage cultured in CSF-1+IL-10. PROK2 RNA was also slightly upregulated in CSF-1-derived macrophage stimulated with LPS.

### Example 42

### PROK2 Secretion by IL-10-Stimulated Human Macrophage Cells

Experiment #1:

Materials and Methods: Monocytes were collected from PBMNC by negative selection from a donor. Monocytic cells were washed 1×, then resuspended in assay media (RPMI-1640, 10% FBS, 2-ME, L-glutamine and sodium pyruvate). Cells at 3×10⁵/ml were then cultured in media containing 50 ng/ml hCSF-1 (R&D Systems, lot#CC105041)+/−50 ng/ml hIL-10 (R&D Systems, #55) using 6-well low adhesion plates (Costar, #3471). Cells were cultured at 5% CO₂, 37° C. for 7 days. On day 4 additional CSF-1 and IL10 were added to cultures in respective wells. On day 6, 100 ng/ml E. coli-derived LPS (Sigma, L-4391, 78H4122) was added to several wells (CSF-1 alone). On day 7 cells supernatants were collected (froze at −20° C.). Macrophage supernatants were assayed by ELISA for PROK2.

Results: ELISA results demonstrated low levels of PROK2 protein constitutively secreted by CSF-1-derived macrophage cells. However, macrophage cocultured in IL-10 and CSF-1 secreted significantly more (6×) PROK2.

Experiment #2

Materials and Methods: Monocytes were collected from PBMNC by negative selection from a donor. Monocytic cells were washed 1×, then resuspended in assay media (RPMI-1640, 10% FBS, 2-ME, L-glutamine and sodium pyruvate). Cells at 2.5×10⁵/ml were then cultured in media containing 100 ng/ml hCSF-1 (R&D Systems, lot#CC105041) using 6-well (3 mls/well) low adhesion plates (Costar, #3471). In addition to CSF-1, some cultures were also supplemented with 10 ng/ml hIL-4 (R&D Systems, #44), 10 ng/ml hIL-10 (R&D Systems, #55) or 10 ng/ml hTGB-b (R&D Systems, #95). Cells were cultured at 5% CO₂, 37° C. for 6 days. On day 5 cells (CSF-1 alone) were stimulated with either 50 ng/ml CpG2006 or 100 ng/ml E. coli-derived LPS (Sigma, L-4391, 78H4122). On day 6, supernatants were collected and assayed by PROK2 ELISA.

Phenotypic analysis was performed by staining cells in FACS buffer (PBS, 3% pooled AB human Serum [Sigma], 0.02% sodium azide and 50 ug/ml huIgG [Zymed]) at 1×10⁵ cells/sample in 50 ul volume for 30 min at 40° C. All mabs purchased from BD Pharmingen and used at 1:20 dilution. Cells were analyzed on the FACS Caliber using Cell Quest software.

Results: As shown previously in experiment #2, there were low levels of PROK2 secreted by macrophage in CSF-1 alone and stimulation with LPS, CpG2006 or IL-4 had no effect. However, coculture with either TGF-b or IL-10 resulted in higher levels of secreted PROK2 (4×).

In addition, phenotypic analysis of the macrophage cells demonstrated a good correlation between CD163 expression and PROK2 levels. CD163, the receptor for hemoglobin-haptoglobin complexes, is regarded as one of the best markers for alternatively-activated macrophage, which are typically associated with tumors (see Am J Surg Pathol, 2005, 29:617 and Cellular Oncology, 2005, 27:203).

### Example 43

### Secretion of PROK2 by Human PMNs

Experiment #1:

Materials and Methods: Human polymorphonuclear neutrophils (PMNs) were collected by first removing lymphocytes from human blood via Ficol density gradients (per above). The PBC and PMN-containing cell pellets were then washed in PBS 1×. Cells were then resuspended in H₂0 to lyse RBCs and then 10×HBSS was added to normalize the osmolarity. This was repeated 1× to remove all residual RBCs. Cells were washed 2×s in PBS, resuspended in assay media (above), then counted using Turks stain.

Culture of PMNs was performed in assay media supplemented with 10 ng/ml hGM-CSF (R&D Systems, #25), 100 ng/ml E. coli-derived LPS (above) or nothing. Cells were seeded into T-25 flasks at 15×10⁶/flask in 8 mls media/flask. Cells were cultured at 5% CO₂, 37° C. for 2 days. RNA from remaining freshly isolated PMNs (40×10⁶) PMNs were analyzed for PROK2 and PROK1 expression. Supernatants from cultured PMNs, as well as the cells (pelleted and frozen) were collected and analyzed for PROK2 and PROK1 expression.

Results: Freshly isolated PMNs expressed relatively high levels of PROK2 RNA, but no ProK1 RNA. Cultured PMNs secreted very low levels of PROK2. Although there was a 2-fold increase in GM-CSF-stimulated PMNs, these levels were still relatively low (<20 pg/ml).

Experiment #2:

Materials and Methods: Purified human PMNs per exp #1 for PROK2 secretion after fMLP stimulation. PB was isolated from a donor. PMNs purified per exp#1 (above). Both lymphocytes (collected from interface) and PMNs were cultured in media (above) with and without 1 uM fMLP (Sigma, 074K1437) for up to 120 minutes. Periodically, supernatants and cells were collected and frozen for future analysis.

Results: fMLP stimulation of PMNs resulted in PROK2 secretion. Little if any PROK2 was detected from lymphocytes cultured with or without fMLP. Thus the primary source of PROK2 from PB cells in PMNs. In addition, maximal proK2 secretion occurred after only 10 minutes in fMLP suggesting it is preformed and ready for release from normal circulating PMNs.

### Example 44

### Secretion of PROK2 by Mouse PMNs

Materials and Methods: Collected femurs from one female Balb/C mouse. Bone marrow (BM) cells were purged using assay media (above) in a syringe and 25-gauge needle. Cells were washed one time, then resuspended at 1×10⁵ cells/ml in media supplemented with 100 ng/ml mSCF (R&D Systems, Minneapolis, Minn.) and 20 ng/ml mG-CSF (R&D Systems). Cells were then cultured in a 24 well plate at 5% CO₂, 37° C. On day 7 cultures were split 1:2 and fresh media was added. On day 9 cells and supernatants were harvested and given to for analysis or PROK2 expression.

Results: By ELISA, mouse PMNs spontaneously secreted low levels of PROK2. In addition, both ProK1 and PROK2 RNA were detected by TaqMan.

### Example 45

### Correlation of PROK2 Expression with Tumor Growth (TUG 03 and 12)

A) Preparation of Cells Expressing Mouse PROK2 or VEGFA into SW620, SW480 or CT-26 Tumor Cells:

Cloning of Mouse PROK2

Full-length mouse PROK2 was PCR amplified out from a template using the Advantage 2 PCR Kit (BD Biosciences). 5′ EcoRI and FseI sites were added using forward primer GATCGAGAATTCGGCCGGCCACCATGGGGGACCCGCGCT (SEQ ID NO: 32). 3′ BamHI and MluI sites were added using reverse primer TCGATCGGATCCACGCGTTCATTTCCGGGCCAAGCA (SEQ ID NO: 33). Amplification conditions were: 94° C.×1 min, [94° C.×15 sec, 68° C.×1 min]×30, 70° C.×5 min, and held at 4° C. Following amplification, reactions were cleaned up using Qiagen PCR Purification Kit according to manufacturers'. instructions. Double digest with EcoRI and BamHI in NEB Buffer #2 were performed on amplified fragment as well as 1 μg of pZKK130 and pZKK131 (B 7294, p. 127-129). After digestion, samples were run out on a 1% agarose gel, correct bands were excised and purified with Qiagen Gel Purification Kit per mfg. instructions. Ligation reactions were set up for each vector and insert using T4 DNA Ligase (Promega). Following o/n incubation at 14° C., 1 μl ligation mixture was transformed into electrocompetent TOP10 E. Coli (Invitrogen) using a standard electroporation protocol. Sequence reports showed that pZKK131-zven1 and pZKK130_zven1 were correctly cloned.

Cloning of Mouse VEGFA

An aliquot of a vector containing the full-length murine VEGFA (164) with flanking 5′FseI and 3′ AscI sites was generated in-house. FseI and AscI restriction digests were performed on the above vector in NEB Buffer #4. FseI and MluI digests were performed on pZKK130-zven1 and pZKK131_zven1 in NEB Buffer #4 (AscI and MluI have compatible ends.) After digestion, samples were run out on a 1% agarose gel, correct bands were excised and purified with Qiagen Gel Purification Kit per manufacturers'. instructions. Ligation reactions were set up for each vector using T4 DNA Ligase. Following o/n incubation at 14° C., 1 μl ligation mixture was transformed into electrocompetent TOP10 E. Coli (Invitrogen) using a standard electroporation protocol. The following day colonies were submitted for sequencing. Sequence reports showed that pZKK131 mvegfa and pZKK130 mvegfa were correctly cloned.

B) Cell Culture

CT26 mouse colon carcinoma (ATCC, Manassas, Va.), SW480 human colorectal adenocarcinoma (ATCC, Manassas, Va.), and SW620 human colorectal adenocarcinoma—lymph node metastatic site (ATCC, Manassas, Va.) cultures were obtained from in-house stocks and expanded according to ATCC protocol. 293FT cells (SV40 T-antigen expressing) were obtained from in-house stocks and expanded in DMEM, 10% FBS, 1× GlutaMax (Invitrogen) according to ATCC protocol.

C) Viral Production

For viral production, 293FT cells were plated in 6-well standard tissue culture plates at a density of 700,000 cells per well and allowed to attach overnight. Transient transfections were set up using FuGene-6 (Roche) according to manufacturer's. protocol at a 3:1 FuGene:DNA ratio. 3 μg DNA was transfected per well consisting of 1 μg retroviral vector construct, 1 μg pVPack gag-pol, and 1 μg pVPack vsvg (Stratagene). The next day the medium was replaced with 1 mL fresh medium and cells were examined for GFP expression by fluorescent microscopy. The following day, retroviral supernatant was collected, filtered through a 0.45 μm syringe filter and immediately used for transduction or frozen at −80° C.

D) Retroviral-Mediated Transduction of Tumor Cell Lines

CT26 cells were plated in 6-well plates at a density of 80,000 per well and allowed to attach overnight. SW620 and SW480 cells were plated in 6-well plates at a density of 100,000 cells per well and allowed to attach overnight. Culture medium was aspirated and replaced with either a 1:2 or 1:10 dilution of above-produced retroviral supernatant containing 4 μg/mL polybrene (Sigma) from 1000× stock. Infection was allowed to proceed overnight. The next day cells were examined for GFP expression and were split 1:3 with one subset placed on puromycin selection at 20 μg/mL for CT26 cells and 1 μg/mL for SW620 and SW480.

E) Analysis of Stable GFP Expression

Initial analysis of stable GFP-producing cells was performed using fluorescence microscopy using an appropriate filter for GFP detection. Subsequent analysis was performed by flow cytometry on a FACSCaliber instrument with CellQuest software (BD Biosciences). Briefly, after 7 days of expansion, cells were harvested by trypsin, washed 2× with PBS, and flow cytometry was performed. FL1 intensity was compared between transduced cells and parentals.

F) Analysis of PROK2 and VEGFA Expression

ELISA's were performed on 72-hour conditioned medium from PROK2 and VEGFA stable producing cell lines with appropriate controls using standard ELISA procedures. VEGFA capture antibody was NF-493 (R&D Systems) and detection antibody was BAF-493 (R&D Systems). Mouse VEGFA from in-house cytokine bank (Lot# RQ018111) was used as a standard. For PROK2, capture antibody was E8588, clone #111 and detection antibody was E8484 which was freshly biotinylated with EZ-Link sulfoNHS-LC-Biotin (Pierce). Human PROK2 (Peprotech, Rocky Hill, N.J.) was used as a standard. ELISA's were read on a SpectraMax instrument and analyzed with SOFTMax Pro.

G) Method for Detecting Human PROK2 Detection ELISA in Conditioned Media

The capture antibody (E8588, clone #111, 1.1 mg/ml) is diluted to 250 ng/ml in ELISA A buffer. The plate is coated with the antibody (100 ul/well in Nunc 96-well ELISA plates) and the plates are sealed and incubated overnight at 4oC. The plates are washed with 250 ul/well 3× in ELISA C buffer, then blocked with ELISA-B (ELISA-C+2% BSA), 200 ul/well and incubated for 15 min at RT, after which the plates are flicked to empty. The plates are washed again with 250 ul/well 3× in ELISA C buffer.

Standard curve dilutions are prepared using PROK2 (Peprotech Prokineticin-2; Stock Concentration=0.919 mg/ml). When measuring PROK2 levels in conditioned medias (CMs), samples are usually tested as is, plus with couple serial dilution points. If the sample volume is limited then the starting dilution is made at lowest possible point (1:1 or 1:2, etc) since protein level is not known. The standard curve and the dilutions of the samples are made in the culture media at the following concentrations: 30.0000 ng/ml (1:3); 10.0000 ng/ml (1:3); 3.3333 ng/ml (1:3); 1.1111 ng/ml (1:3); 0.3704 ng/ml (1:3); 0.1235 ng/ml (1:3); 0.0412 ng/ml (1:3); 0.0137 ng/ml (1:3); 0.0046 ng/ml (1:3); 0.0015 ng/ml (1:3); 0.0005 ng/ml (1:3); and Diluent only.

The samples and the standards (100 ul/well) are added to the plate and incubate on a plate shaker for 1.5 hours at 37° C., then washed 3× in ELISA C buffer, 250 ul/well. The antibody (E8484 clone #124, 1.32 mg/ml) is freshly biotinylated by adding 2.5 ug of antibody for each plate (for 2 plates=3.79 uL of antibody), 1 uL of 1 mg/ml Biotin (EZ-Link sulfoNHS-LC-Biotin, PIERCE) per ug of antibody (for 2 plates=5 uL) is added, and incubated at room temperature for 45 minute (mixing at low speed). The biotinylation reaction is stopped by adding 50 uL of 2M glycine, and the volume is brought to desired amount with ELISA B (for 2 plates=20 ml). The biotinylated antibody is used at a concentration of 250 ng/ml. One hundred uL/well is added and the plates are incubated for 1.5 hr, 37° C. The plates are washed 3× in ELISA C, 250 ul/well.

SA-HRP is diluted to 1:3000 in ELISA B and plated at SA-HRP, 100 ul/well and incubated for 1 hr at 37° C. The plates are washed 3× in ELISA C buffer, 250 ul/well. and TMB solution is added at 100 ul/well. The plates are developed for 3 minutes at RT, on the bench. Color development is stopped by plating BioFX 450 Stop reagent, 100 ul/well, and read at OD at 450 nm, within 15 minutes of stop.

H) Preparation of Cells for In Vivo Use

Stably-transduced CT26 cells from pZKK131_empty, pZKK131_zven1, and pZKK131_mvegfa were expanded, trypsinized, washed 2× with PBS, passed through a 40 μm cell strainer, counted by trypan blue exclusion, and diluted to a concentration of 2 million cells per mL. These were placed on ice prior to inoculation in mice.

Stably-transduced SW620 and SW480 cells from pZKK131_empty, pZKK131_zven1, and pZKK131_mvegfa were expanded, trypsinized, washed 2× with PBS, passed through a 40 μm cell strainer, counted by trypan blue exclusion, and diluted to a concentration of 10 million cells per mL. These were placed on ice prior to inoculation in mice.

I) Injection of Transfected Cells into Mouse Tumor Model

Pooled SW620, SW480 and CT-26 tumor cells transfected with retrovirus carrying PROK2+GFP, VEGFA+GFP, or GFP alone were tesed in this study. Treatment groups were injected with an inoculum of 0.5×10⁶ cells for the SW620 or SW480 cell lines and 0.1×10⁶ cells for the CT-26 cell line as described in Table 20, below. Each animal received 50 uL solution of cells using a 0.5 mL insulin syringe with a 30G needle. The injections were given into the mammary fat pad. Tumor measurements (length and width in mm) were made with a digital caliper and recorded once the size exceeded 10 mm square. Blood was collected for a CBC on all animals prior to beginning the study, on day 7 and at study termination. Animals were euthanized at the discretion of the study monitor as the tumors reached a given size or the tumors are ulcerating the skin. At the time of euthanasia, blood was collected for CBC and serum, tumor tissue collected for RNA analysis (frozen on dry ice) and histology (10% NBF for 24 hrs then into 70% ETOH) and the spleen collected for histology. ELISA assay for PROK2 and VEGFA was performed on the serum. RNA from each tumor was isolated and assayed for PROK2 and VEGFA expression by Taqman RTPCR.

TABLE 20
Study Groups

| Group | | | | |
|---|---|---|---|---|
| Number | N/Group | Mouse strain | Inoculum | Blood draws |
| 1 | 10 | Nu/Nu | SW620 + GFP | Pre, day 7, end |
| 2 | 10 | Nu/Nu | SW620 + | Pre, day 7, end |
| | | | PROK2 + GFP | |
| 3 | 10 | Nu/Nu | SW620 + | Pre, day 7, end |
| | | | VEGFA + GFP | |
| 4 | 10 | Nu/Nu | SW480 + GFP | Pre, day 7, end |
| 5 | 10 | Nu/Nu | SW480 + | Pre, day 7, end |
| | | | PROK2 + GFP | |
| 6 | 10 | Nu/Nu | SW480 + | Pre, day 7, end |
| | | | VEGFA + GFP | |
| 7 | 10 | BALB/c | CT-26 + GFP | Pre, day 7, end |
| 8 | 10 | BALB/c | CT-26 + | Pre, day 7, end |
| | | | PROK2 + GFP | |
| 9 | 10 | BALB/c | CT-26 + | Pre, day 7, end |
| | | | VEGFA + GFP | |

J) Expression of PROK2 or VEGFA in Mouse Model

Expression of PROK2 and VEGFA was measured by Taqman PCR.

Results: PROK2 expression in CT26 tumors lead to an increase in tumor growth similar to overexpression of VEGFA.

Note: the SW620 and SW480 cells failed to establish tumors in all test groups. This was not an effect of either PROK2 or VEGFA.

Experiment #2:

Materials and Methods: In a second experiment the CT26 cells were prepared according to A) above.

Ten nu/nu mice per group (Lot #1416) were injected with an inoculum of 150,000 transfected cells. The three groups were GFP-only, GFP plus VEGF, GFP plus PROK2. In addition, a fourth group was inoculated with parental (non-transfected CT26 cells). Each animal received the 50 uL solution of cells using a 0.5 mL insulin syringe with a 30G needle. The injections were given into the mammary fat pad. Tumor measurements (length and width in mm) were made with a digital caliper and recorded once the size exceeded 10 mm square. Blood was collected for serum at study termination. Animals were euthanized at 21 days or earlier if tumors reached a given size or the tumors were ulcerating the skin. At the time of euthanasia, blood was collected for serum (ELISA for VEGF and PROK2), tumors were collected and weighed, tumors were then split into tissue samples for RNA analysis (frozen on dry ice) and for histology (10% NBF for 24 hrs then into 70% ETOH). The spleen was also collected for histology. ELISA assay for PROK2 and VEGFA was performed on the serum. RNA from each tumor was collected and assayed for PROK2, VEGF-A and GPR73a and 73b expression.

Results: Increased tumor growth was seen in mice carrying tumors overexpressing PROK2 and VEGFA, consistent with the previous results seen in the BalbC mice.

### Example 46

### Angiogenic Evaluation of PROK2 Utilizing the Rabbit Corneal Micropocket Model (ANG 19)

Materials and Methods: Twenty-five New Zealand White Rabbits were used as test subjects. Five groups of five animals per group were used. The negative control group had a methacrylate/sucralfate pellet dosed with saline inserted into a surgically created corneal micropocket. VEGF was used as a positive control. The VEGF group had a methacrylate/sucralfate pellet containing 100 ng VEGF inserted into a surgically created corneal micropocket. The PROK2 groups had a methacrylate/sucralfate pellet containing either 1 ng or 10 ng PROK2 inserted into a surgically created corneal micropocket. All the animals were examined on day 6 and day 9.

Results: PROK2 (10 ng dose) caused a significant increase in angiogenesis when compared to control animals and also to the lower dose PROK2. This response was seen in all five treated animals.

### Example 47

### Angiogenic Evaluation PROK2 Transfected SW620 Cells in Diffusion Chamber Model in Nude Mice

Experiment #1 (ANG 05)

Materials and Methods: Nude mice were used as test subjects. Three groups of five animals per group were used for this study. The negative control group received a diffusion chamber loaded with 1×10⁶ non-transfected SW620 cells in 200 uL implanted subcutaneously in the mid back. The positive control group received a diffusion chamber loaded with 1×10⁶ SW620 cells transiently transfected with VEGF in 200 uL implanted subcutaneously in the mid back. The experimental group received a diffusion chamber loaded with 1×10⁶ SW620 cells transiently transfected with PROK2 in 200 uL implanted subcutaneously in the mid back. The animals were euthanized on day 7 following the implantations and the skin in contact with the chambers dissected away from the chamber and photographed. Blood was also collected for serum for ELISA assays.

Results: Animals in the VEGF as well as PROK2 group showed increased vascularity and microvascular leakage suggesting an angiogenic effect of PROK2.

Experiment #2 (ANG16)

Materials and Methods: Female Nude mice (n=30) are used as test subjects. Three groups of ten animals per group are implanted with chambers containing cells. Each diffusion chamber is loaded with approximately 200 uL of saline or cells at a concentration of 5.0×10⁶ cells per mL and implanted subcutaneously in the mid back. The animals are euthanized on day 7 following the implantations and the skin in contact with the chambers dissected away from the chamber and photographed. Fluid from each diffusion chamber is collected at euthanasia and assayed for cellular viability.

### Example 48

### Angiogenic Evaluation of PROK2 Secreted by Stably Transfected RENCA.2 Cell in the Diffusion Chamber Model in Nude Mice

Experiment #1: (ANG 06)

Materials and Methods: Female Nude mice (n=40) are used as test subjects for this study. Eight groups of five animals per group are implanted. Three clones of RENCA.2 cells containing the empty vector, three clones of RENCA.2 cells expressing PROK2 and two clones of RENCA.2 cells expressing VEGFA will make up the study groups. Each diffusion chamber will be loaded with 170-180 uL of cells at a concentration of 5×106 cells per mL and implanted subcutaneously in the mid back. Half of the animals will be bled for serum on day 1 and the other half bled for serum on day 2 and the serum assayed for protein levels. The animals will be euthanized on day 7 following the implantations and the skin in contact with the chambers dissected away from the chamber and photographed. Blood will also be collected for serum for ELISA assays for protein levels on day 7. Following the photographic procedure, the surface of the skin in contact with the diffusion chamber will scraped and the material collected and assayed for Hb levels.

Experiment #2: (ANG 07)

Materials and Methods: Female Nude mice (n=38) are used as test subjects. Seven groups of five animals per group are implanted with chambers containing cells. One group of 3 animals with chambers containing saline only is implanted. Three clones of RENCA.2 cells containing the empty vector and three clones of RENCA.2 cells expressing PROK2 will make up the study groups with the saline acting as the true negative control. Each diffusion chamber is loaded with 170-180 uL of saline or cells at a concentration of 0.5×106 cells per mL and implanted subcutaneously in the mid back. The animals are euthanized on day 9 following the implantations and the skin in contact with the chambers dissected away from the chamber and photographed. Blood is also collected for serum for ELISA assays for protein levels on day 9. Fluid from each diffusion chamber is collected at euthanasia and assayed via ELISA for protein levels.

### Example 49

Dose Ranging Study to Evaluate the Angiogenic Potential of Retrovirus-Transfected CT-26 Cells in the Diffusion Chamber Model in Nude Mice (ANG17)

Materials and Methods: Female Nude mice (n=20) are used as test subjects for this study. Four groups of five animals per group are implanted with chambers containing cells. The diffusion chambers are loaded with approximately 200 uL of cellular suspension at a concentration of 2.5×10⁶ cells per mL or 10×10⁶ cells per mL and implanted subcutaneously in the mid back. The animals are euthanized on day 7 following the implantations and the skin in contact with the chambers dissected away from the chamber and photographed. Fluid from each diffusion chamber is collected at euthanasia and assayed for cellular viability and VEGF levels.

### Example 50

### Angiogenic Potential Measured in Matrigel Model (ANG12)

Materials and Methods: Matrigel (low growth factor, Cat #47743-722) is injected s.c., 400 uL/site, bi-laterally and dorsally in C57/B6 female mice. Two sites of injection are performed on each mouse with the left being always Control. Ten mice are tested in each group. Matrigel, +/− factors, was adjusted to contain +/−60Units/mL heparin (Sigma). Heparin is prepared at 50× in PBS on same day and passed through 0.2 micron filter.

### Example 51

### PROK2 Induces the Release of VEGF from Wky12-22 Cells

The rat cell line Wky12-22, derived from the neo-intima of 12 day old rats, expresses predominately the PROK2 receptor GPCR73a while the corresponding control cell line Wky3m, from 3 month old rats does not express either receptor:

Wky12-22 cells treated with PROK2 secrete the pro-angiogenic chemokine GRO alpha in a time and dose dependent fashion. Secretion of other angiogenic factors, such as VEGF, were analyzed to see if they were secreted in response to PROK2.

At 24 hours, GRO concentrations of 1.5 ng/ml were obtained with a 1 nM PROK2 concentration. GRO is detected in the media as early as 2 hours post treatment and reaches maximal levels at 20 hours post treatment. Wky3m cells treated with PROK2 do not secrete GRO alpha.

A more inclusive rat cytokine/chemokine screen was conducted on Wky12-22 cells treated with PROK2 for 24-48 hours. Conditioned media were screened for 65 analytes using a Luminex based system performed by RBM (Rules Based Medicine, Inc. 3300 Duval Rd, Austin, Tex. 78759). Based on these results, which indicated an increase in both GRO and VEGF at 24 hours, a second experiment was run with both 24 and 48 hour time points.

Materials and Methods: Wky12-22 cells, were plated on a Falcon 24 well plate and grown to 95% confluency in DMEM+10% FBS in 5% CO2 37 degree C. incubator. Cells were treated with 0.5 ml PROK2 in either serum containing or serum free media (RPMI 1640+/−5% FBS). The plate was decanted and 0.5 ml/well media containing PROK2 at 1, 10, and 10 ng/ml was added to each well and conditioned media was collected following either a 24 hour incubation or a 48 hour incubation Basal wells containing media only were also run. CM was analyzed by ELISA for VEGF levels.

Results: PROK2 induced the release of VEGF from Wky12-22 cells. Maximal levels were observed in the presence of 5% serum and at PROK2 concentrations of 1 nM (10 ng/ml). An approximate 2 fold increase of VEGF was observed. See Table 21 below.

TABLE 21

| Time | Basal | 1 nM PROK2 |
|---|---|---|
| 24 hours | 90 pg/ml | 180 pg/ml |
| 48 hours | 160 pg/ml | 280 pg/ml |

### Example 52

### PROK2 Expression in Human Cancer Sera

PROK2 gene expression is increased in colon cancer tissues compared to normals. To determine if this increased expression might correlate with an increase in PROK2 protein concentration in cancer patients, PROK2 levels in sera obtained from cancer patients was compared with age and sex matched control donors.

Method: Human cancer patient sera and control matched donor sera were purchased from ProMedDx, LLC, (Norton, Mass.) and from Asterand ( ). Sera from the following types of cancer was obtained and screened: Liver, lung, ovary, breast, pancreas, colon, brain, bladder, kidney and thyroid. Samples were thawed and diluted to 1% and assayed by ELISA.

Method for detecting human PROK2 Detection ELISA in serum:

Dilute capture antibody (E8588, clone #111, 1.1 mg/ml) to 250 ng/ml in ELISA A buffer. Plate coating antibody, 100 ul/well in Nunc 96-well ELISA plates. Seal plates and incubate overnight at 4° C. Wash 3× in ELISA C buffer, 250 ul/well. Block plate with ELISA-B (ELISA-C+2% BSA), 200 ul/well. Incubate 15 min, RT. Flick plate to empty. Wash 3× in ELISA C buffer, 250 ul/well. Standard Curve Dilutions are as follows: PROK2 standard: PROK2 (Peprotech, Rocky Hill, N.J.) Stock Concentration=0.919 mg/ml; Primary Dilution was 1:100 in ELISA-B; Dilutent wasl % normal human serum pool (prepared from ProMedDx normal donors). Concentrations were as follows: 30.0000 ng/ml, 10.0000 ng/ml; 3.3333 ng/ml; 1.1111 ng/ml; 0.3704 ng/ml; 0.1235 ng/ml; 0.0412 ng/ml; 0.0137 ng/ml; 0.0046 ng/ml; 0.0015 ng/ml; 0.0005 ng/ml; and Blank. Add samples and the standards (100 ul/well) to the plate and incubate on a plate shaker for 1.5 hours at 37° C. Wash 3× in ELISA C buffer, 250 ul/well. Biotinylate E8484 (clone #124, 1.32 mg/ml) freshly and use it at 250 ng/ml concentration. Add 100 uL/well. Incubate for 1.5 hr, 37° C. Biotinylation: Use 2.5 ug of antibody for each plate (for 2 plates=3.79 uL of antibody).

Add 1 uL of 1 mg/ml Biotin*** per ug of antibody (for 2 plates=5 uL). Incubate at room temperature for 45 minute (mixing as low speed). Stop biotinylation by adding 50 uL of 2M glycine. Bring volume to desired amount with ELISA B (for 2 plates=20 ml). Add EZ-Link sulfoNHS-LC-Biotin, PIERCE, #21335. Wash 3× in ELISA C, 250 ul/well. Dilute SA-HRP to 1:3000 in ELISA B. Prepare 10 ml/plate. Plate SA-HRP, 100 ul/well. Incubate for 1 hr, 37° C. Add SA-HRP. Take out a needed volume of TMB from fridge and allow warm to RT in the dark (10 ml/plate). Wash 3× in ELISA C buffer, 250 ul/well. Plate TMB solution, 100 ul/well. Develop plates for 5 minutes, RT, on the bench. Stop color development by plating BioFX 450 Stop reagent, 100 ul/well. Read plates, OD at 450 nm, within 15 minutes of stop. Set the wavelength correction to 540 nm in order to correct for optical imperfections on the plate.

Results: are shown in Table 22 below.

TABLE 22

| | PROK2 | | | PROK2 |
|---|---|---|---|---|
| | positive/total | PROK2 | | concentration |
| | number of cancer | concentration | | In matched donor |
| Type of cancer | sera tested | ng/ml | | control ng/ml |
| Liver | 1/9 | 1.4 | ng/ml | 0 |
| Thyroid | 1/2 | 3.1 | ng/ml | 0 |
| Lung | 4/18 | 0.3-0.8 | ng/ml | 0 |
| Colon | 1/10 | 0.6 | ng/ml | 0 |
| Breast | 3/12 | 0.4-0.6 | ng/ml | 0 |
| Bladder | 2/3 | 0.3-0.4 | ng/ml | 0 |
| Ovary | 2/5 | 0.233-3.0 | ng/ml | 0 |
| Brain | 0/4 | <0.3 | ng | 0 |
| Pancreas | 0/5 | <0.3 | ng/ml | 0 |
| Kidney | 0/2 | <0.3 | ng/ml | 0 |

Staging values at diagnosis and at the time of the serum collection for the 4 highest lung cancer patients were: IV, I, IV, IV respectively. All Colon cancer patients were Stage IV, the 2 highest PROK2 bladder cancer serums were from patients with tumors at Grade 2 and 3, the patient with no circulating PROK2 was a T1. All of the PROK2 positive ovarian cancer patients were Grade IIIC. No staging data was available for the liver and thyroid cancer patients.

This data suggests that PROK2 levels are elevated in some cancer patients, with the highest levels detected in thyroid, liver and lung cancer. The highest incidence of PROK2 presence was in the lung cancer patients

### Example 53

### PROK2 Direct Effects on Tumor Cells

A) PROK2 Effects on 4Ti1.2 Murine Breast Cancer Cells

4T1.2 murine breast cancer cells were tested for signaling in response to PROK2 using the Phospho-protein assay.

On day 1 4T1.2 murine breast cancer cells were plated out at 1×104 cells/well in complete growth media in 96-well, flat-bottom tissue culture plates. On day 2 cells were switched into serum free media for overnight starvation. On day 3 serial dilutions of PROK2 ranging from 1-100 ng/ml were added to the cells in serum free media containing 0.5% BSA and incubated at 37° C. for 7 and 15 minutes.

Following incubation, cells were washed with ice-cold wash buffer and put on ice to stop the reaction according to manufacturer's instructions (BIO-PLEX Cell Lysis Kit, BIO-RAD Laboratories, Hercules, Calif.). Wash buffer was removed prior to adding 50 μL/well lysis buffer to each well; lysates were pipetted up and down five times while on ice, then agitated on a microplate platform shaker for 20 minutes at 300 rpm and 4° C. Plates were centrifuged at 4500 rpm at 4 oC for 20 minutes. Supernatants were collected and transferred to a new micro titer plate for storage at −20° C.

Capture beads (BIO-PLEX Phospho-ERK1/2 and JNK Assay, BIO-RAD Laboratories) were combined with 50 μL of 1:1 diluted lysates and added to a 96-well filter plate according to manufacture's instructions (BIO-PLEX Phosphoprotein Detection Kit, BIO-RAD Laboratories). The aluminum foil-covered plate was incubated overnight at room temperature, with shaking at 300 rpm. The plate was transferred to a microtiter vacuum apparatus and washed three times with wash buffer. After addition of 25 μL/well detection antibody, the foil-covered plate was incubated at room temperature for 30 minutes with shaking at 300 rpm. The plate was filtered and washed three times with wash buffer. Streptavidin-PE (50 μL/well) was added, and the foil-covered plate was incubated at room temperature for 15 minutes with shaking at 300 rpm. The plate was filtered and washed two times with bead resuspension buffer. After the final wash, beads were resuspended in 125 μL/well of bead suspension buffer, shaken for 30 seconds, and read on an array reader (BIO-PLEX, BIO-RAD Laboratories) according to the manufacture's instructions. Data was analyzed using analytical software (BIO-PLEX MANAGER 3.0, BIO-RAD Laboratories). Increases in the level of the phosphorylated ERK1/2 and JNK transcription factors present in the lysates were indicative of a receptor-ligand interaction.

A small (1.8×) increase in ERK phosphorylation was detected, with maximal response seen at 100 ng/ml PROK2 and at the 5 minute time point. This response was dose dependent and time dependent. The 4T1.2 line was subcloned and screened again for ERK activity. A sub clone F9 was identified that responded at the same 1.8× level. This line was further tested for PROK2 effects using an Alamar Blue based proliferation assay. Other receptor expressing cancer lines also tested for PROK2 induced proliferation were: LL2, IMR-32 and CT26.

Prior to treating with PROK2, assay conditions were optimized. Cells were plated at varying concentrations and incubated with Alamar blue for varying times to determine optimal conditions.

The final experiment was done in 0 and 1% serum in DMEM media with PROK2 at 0, 1, and 10 ng/ml. Cells were plated in 96 well plates on Day 0 in their regular growing media (DMEM+10% FBS) at a concentration off 1000 cells/well. The following day, plates are decanted and PROK2 in either DMEM only or DMEM+1% FBS was added to cells, 100 ul/well. 24 hours later, 10 ul of Alamar Blue (Alamar Biosciences, Inc. 4110 N. Freeway Blvd., Sacramento, Calif. 95834-1219) was added to each well. In order to eliminate edge effects, the outside edge wells were not used. Background values were obtained from wells containing media only. N=4/treatment condition.

Readings were taken on days 1, 2, and 3 post PROK2 treatment. Plates were read on a Cytofluor fluorometric plate reader, excitation wavelength 530 and emission wavelength 580 following a 2 hour incubation in Alamar blue. These conditions yielded values that were on the linear portion of the curve, indicating the cells were still in log phase and sufficient substrate was present.

Results: Following a 3 day incubation, in the 4T1.2 F9 cells only, increased proliferation was detected at both 0 and 1% serum conditions. (n=2 experiments). Cells treated with PROK2 for 3 days had an approximate 36% increase in cell #, based on Alamar blue readings in n=2 experiments (30% and 46% respectively). This effect was dose dependent with the largest effect seen at the 100 ng/ml concentration.

B) PROK2 Effects on Wky12-22 Murine Breast Cancer Cells

The same protocol as specified in part A) of this example, was used but Wky12-22 cells were substituted for the 4T1.2 cells.

Results: A maximal ERK 1/2 response (11× fold induction over basal) was seen 15 minutes post treatment with 100 ng/ml PROK2. The JNK pathway was also activated with a maximal response of 3× at 15 minutes post 100 ng/ml PROK2 treatment. Both responses were dose and time dependent.

When receptor expression using Taqman RTPCR was performed on the Wky12-22 cells, the ratGPR73a was expressed at 17.821% of GUS and ratGPR73b was expressed at 0.010% of GUS. In addition, ratGPR73a was expressed at 5.555% GAPDH and ratGPR73b was expressed as 0.003% of GAPDH.

### Example 54

### Neutralization of PROK2 by Purified Anti-PROK2 Monoclonal Antibodies as Measured by Reporter Assay

Neutralization of PROK2 activity as measured by the Luciferase based PROK2 Activity Assay described in Example 36 above was performed by antibodies from hybridomas which were allowed to grow in serum-free media.

Results: The EC50 results are shown in Table 23, below. All EC50 values are in the nanomolar range in this assay. These antibodies are free of contaminating bovine IgG.

TABLE 23

| | Antibody | | | |
|---|---|---|---|---|
| | 279.126.5.6.5 | 279.124.1.4 | 279.121.7.4 | 279.111.5.2 |
| EC50 ng/ml | 94.96 | 118.8 | 175.0 | 205.5 |
| Ranking in | #1 | #2 | #3 | #4 |
| order of | | | | |
| Neutrali- | | | | |
| zation | | | | |
| Potency | | | | |

### Example 55

### Neutralization of PROK2 by Purified anti-PROK2 Monoclonal Antibodies as Measured by GROα Inhibition

Neutralization of PROK2 activity as measured by inhibition of GROα secretion as described in Example 32 above was performed by antibodies from hybridomas which were allowed to grow in serum-free media.

Results: The EC50 results are shown in Table 24, below. All EC50 values are in the picomolar range in this assay.

TABLE 24

| | Antibody | | | |
|---|---|---|---|---|
| | 279.126.5.6.5 | 279.124.1.4 | 279.121.7.4 | 279.111.5.2 |
| EC50 ng/ml | 0.64 | 11.39 | 9.33 | 22.88 |

### Example 56

### Neutralizaion of PROK1 by Purified Anti-PROK2 Monoclonal Antibodies as Measured by Reporter Assay

An assay measuring the ability of the antibody produced by hybridoma clones 279.126.5.6.5, 279.124.1.4, 279.121.7.4, and 279.111.5.2, which were allowed to grow in serum-free media, was performed similar to the PROK2 ligand challenge using the reporter assay as described in Example 32, with the exception that the PROK1 ligand challenge was at 30 ng/ml.

Results: In this assay, all four antibodies showed some inhibitory activity within a range of inhibition of 27% to 35%.

### Example 57

### Neutralization of PROK1 by Purified Anti-PROK2 Monoclonal Antibodies as Measured by GROα Release

Purified antibodies from hybridoma clone number 279.126.5.6.5, which was allowed to grow in serum-free media, was used to measure IC50 as a measure of inhibition of PROK1 activity. The reporter assay as described in Example 32 was used. The PROK1 ligand challenge was 200 picomolar.

Results: In this assay the IC50 was determined to be about 3.4 ug/ml.

### Example 58

### Evaluation of PROK2 on Tumor Growth

Three groups of 10 animals (female BALB/c mice) per group were used for this study. Tumors were established on Day 0, by injection of 4T1.2 cells (100 k/mouse) into the mammary fat pad. Cells were prepared similar to methods described above.

The test antibody used was from clone number 279.126.5.6.5 (Lot # E8487 at 1.36 mg/ml), was made up in saline and injected at a dose of 0.5 mg/kg (10 ug/20 g mouse) in 100 uL volume. An IgG1 isotype control mouse monoclonal antibody from R&D Systems (clone 11711.11; Cat. # MAB002; Lot # 1X155101) was used and made up to 100 ug/ml in saline.

Treatment groups were: 1) Saline; 2) Control AB (10 ug/mouse); and 3) Test AB (10 ug/mouse). Tumor growth was measured 3 times/week and PROK2 level in serum was determined at the end of the study. Tumor weights were determined at the end of the study. PROK2, GPR73a and b, and VEGF-A RNA was analyzed by ELISA as described above. Treatments were administered by i.v. injection.

Results: Administration of the anti-PROK2 antibody in this model resulted in a reduction of PROK2 in serum as compared to the saline and control antibody.

### Example 59

### PROK2 is Upregulated in Lung Metastases as Compared to Primary Tumors

4T1.2 tumors were grown in female Balb/C (vendor CRL) for collection of tumor RNA and peripheral blood (PB) samples for complete blood counts (CBCs) and plasma. The 4T1.2 cells were cultured as described above, harvested and washed in PBS twice, then resuspended in cold PBS to 2×106/ml. Fifteen mice were injected with 50 ul volume (1×105 cells) of cells via SC at mammary fat pad (abdomen).

Tumor dimensions were scored starting day 7 and every few days thereafter until termination of study. Mice were weighed once weekly, until day 18. Six mice were sacrificed on days 14, and 21, and 0.5 mls of PB was collected by cardiac puncture and dispensed into EDTA collection tubes. The mice were weighed. The spleens and lungs were removed for separate weight determinations. Tumors were collected for IHC by excising the tumor mass, but leaving the connective tissue (skin and peritoneal wall) in place. Lung tissue was also included from respective mice (preferably with tumor mass). Tumors were fixed in 10% buffered formalin for 24 hours before processing. For the remaining three mice, tumor tissue was collected for RNA analysis: all connective tissue and obvious necrotic tissue was removed then the tumor tissue was snap-frozen in liquid nitrogen on dry ice. This process was repeated with excised lung metastases. The tumor samples were stored frozen at −80oC.

CBCs from the PB samples were acquired using the Hemavet 2500. The PB samples were spun down to acquire plasma samples. PB was also collected from normal non-tumor-bearing mice as control tissues (weigh bodies and spleens as well).

Results: PROK2 levels are upregulated in the lung metastases as compared to the primary tumors. See Table 25 below.

TABLE 25

| | | ProK2 | ProK2 | GPR73a | GPR73a | GPR73b | GPR73b |
|---|---|---|---|---|---|---|---|
| | | expression | expression | expression | expression | expression | expression |
| SAMPLES: | | % of GUS | StDev | % of GUS | StDev | % of GUS | StDev |
| 4T1.2 in | | 0.00 | 0.00 | 0.89 | 0.00 | 0.00 | 0.00 |
| vitro | | | | | | | |
| Early | ~day 14 | 0.05 | 0.04 | 0.74 | 0.04 | 4.51 | 0.04 |
| tumor | | | | | | | |
| (n = 3) | | | | | | | |
| Lung Mets | ~day 28 | 0.61 | 0.44 | 2.08 | 0.44 | 0.51 | 0.44 |
| (n = 3) | | | | | | | |
| B1/6 | | 0.00 | 0.00 | 3.78 | 0.00 | 0.01 | 0.00 |
| Normal | | | | | | | |
| Lung | | | | | | | |

This result coupled with the ability of PROK2 antagonists to reduce the levels of circulating PROK2 in serum, as shown in Example 58 indicates that PROK antagonist will be useful in preventing, limiting, inhibit, or reducing metastasis of a tumor. Thus, PROK antagonists can be used as treatment to prevent, limit, inhibit or reduce metastasis from a primary tumor to a secondary sight of tumor growth.

### Example 60

### Antibodies from Hybridomas 279.111.5.2 and 279.124.1.4 Bind Mouse PROK2 in Addition to Human PROK2

Monoclonal antibodies from separate epitope bins are frequently useful for developing a sandwich ELISA for the detection and quantification of the antigen to which they bind. Based on the epitope binning results described in Example 34, monoclonal antibodies from hybridoma 279.124.1.4, 279.126.5.6.5, 279.121.7.4 were paired with the monoclonal antibody from hybridoma 279.111.5.2 to evaluate their potential for a sandwich ELISA for PROK2. All the pairs detected human PROK2 well and a sensitive detection ELISA for human PROK2 was developed using immobilized monoclonal antibody from hybridoma 279.111.5.2 as the capture antibody and a biotinylated form of the monoclonal antibody from hybridoma 279.124.1.4 as the detection antibody. This sandwich ELISA accurately measures human PROK2 concentrations in both cell supernatants and in serum. In addition to detecting human PROK2, this sandwich ELISA can measure endogenous (mouse) PROK2 concentrations in either the supernatants from (mouse) PROK2 secreting cells and in mouse serum. This observation demonstrates that the monoclonal antibodies from hybridomas 279.111.5.2 and 279.124.1.4 bind mouse PROK2 in addition to human PROK2.

### Example 61

### PROK2 Ligand and Receptor Gene Expression in Cancer Cell Lines

The Taqman RTPCR protocol as described in Example 37 was used to measure RPOK2 ligand and receptors GPR73a, and GPR73b, in various cell lines and in in vitro tumor models.

Results: See Table 26, below.

TABLE 26

| CELL | ProK2 | ProK2 | GPR73a | GPR73a | GPR73b | GPR73b |
|---|---|---|---|---|---|---|
| LINES | In vitro | in vivo | in vitro | in vivo | in vitro | in vivo |
| CT26 | .− | .− | .+++ | .++ | .− | .+ |
| 4T1.2 | .− | .− | .+ | .− | .− | .+++ |
| LL/2 | .− | .− | .++ | .+ | .− | .− |
| IMR-32 | .++ | ND | .++ | ND | .− | .− |
| DLD-1 | .− | .− | .− | .+ | .− | .++ |
| HT-29 | .− | .− | .− | .− | .− | .++ |
| KG-1 | .++++ | ND | .++ | ND | .− | ND |
| TF-1 | .++ | ND | .+ | ND | .− | ND |
| SK-N-SH | .− | ND | .− | ND | .+++ | ND |
| A-673 | .+ | ND | .++++ | ND | .− | ND |

CT26 is a mouse colon carcinoma cell line; 4T1.2 is a murine breast cancer cells; LL/2 is a mouse lung carcinoma cell line; IMR-32 is a human nueroblastoma cell line; DLD-1 is a cell line derived from a human colorectal adenocarcinoma; HT-29 is a human colon adenocarcinoma cell line; KG-1 is a myelogenous leukaemia cell line; TF-1 is a factor-dependent human erythroleukemic cell line; SK-N-SH is a human neuroblastoma cell line; and A-673 is a Human rhabdomyosarcoma cell line.

This data show that these cancer cell lines express the receptor for PROK2 and that the gene for the PROK2 ligand is also present in the IMR-32 and KG-I cell lines.

In addition the data suggest that for some of the cells receptor expression is upregulated in vivo as compared to in vitro expression.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. An antibody that specifically binds a polypeptide comprising the amino acid sequence of SEQ ID NO: 2, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859).

2. The antibody of claim 1, wherein the hybridoma is selected from:
a) the hybridoma of clone designation number 279.124.1.4 (ATCC Patent Deposit Designation PTA-6857);
b) the hybridoma of clone designation number 279.126.5.6.5 (ATCC Patent Deposit Designation PTA-6858); and
c) the hybridoma of clone designation number 279.121.7.4 (ATCC Patent Deposit Designation PTA-6859).

3. The antibody of claim 1, wherein the hybridoma is hybridoma of clone designation number 279.124.1.4 (ATCC Patent Deposit Designation PTA-6857).

4. The antibody of claim 1, wherein the hybridoma is hybridoma of clone designation number 279.126.5.6.5 (ATCC Patent Deposit Designation PTA-6858).

5. The antibody of claim 1, wherein the hybridoma is hybridoma of clone designation number 279.121.7.4 (ATCC Patent Deposit Designation PTA-6859).

6. The antibody of claim 1, wherein the hybridoma is hybridoma of clone designation number 279.111.5.2 (ATCC Patent Deposit Designation PTA-6856).

7. The antibody of claim 1, wherein the antibody is capable of binding the polypeptide as shown in SEQ ID NO: 5.

8. A method of reducing, inhibiting or preventing angiogenesis comprising admixing an antibody with a polypeptide as shown in SEQ ID NO: 2, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

9. The method of claim 8 wherein the binding of the antibody to the polypeptide inhibits, reduces or prevents signal transduction by the polypeptide on its receptor.

10. The method of claim 9 wherein the antibody neutralizes the signal transduction.

11. The method of claim 8 wherein there is also an inhibition of chemokine release.

12. The method of claim 11, wherein the chemokine is GROUα.

13. A method of reducing, inhibiting or preventing angiogenesis comprising admixing an antibody with a polypeptide as shown in SEQ ID NO: 5, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

14. A method of reducing, inhibiting or preventing tumor formation or tumor size comprising admixing an antibody with a polypeptide as shown in SEQ ID NO: 2, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

15. The method of claim 14 wherein the binding of the antibody to the polypeptide inhibits, reduces or prevents signal transduction by the polypeptide on its receptor.

16. The method of claim 15 wherein the antibody neutralizes the signal transduction.

17. The method of claim 14 wherein there is also an inhibition of chemokine release.

18. The method of claim 17, wherein the chemokine is GROα.

19. A method of reducing, inhibiting or preventing tumor formation or tumor size comprising admixing an antibody with a polypeptide as shown in SEQ ID NO: 5, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

20. A method of decreasing vascular leakage comprising admixing an antibody with a polypeptide as shown in SEQ ID NO: 2, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

21. The method of claim 20 wherein the binding of the antibody to the polypeptide inhibits, reduces or prevents signal transduction by the polypeptide on its receptor.

22. The method of claim 21 wherein the antibody neutralizes the signal transduction.

23. The method of claim 20 wherein there is also an inhibition of chemokine release.

24. The method of claim 23, wherein the chemokine is GROα.

25. A method of decreasing vascular leakage comprising admixing an antibody with a polypeptide as shown in SEQ ID NO: 5, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

26. A method of inhibiting, reducing or preventing metastasis formation comprising admixing an antibody with a polypeptide as shown in SEQ ID NO: 2, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

27. The method of claim 26 wherein the binding of the antibody to the polypeptide inhibits, reduces or prevents signal transduction by the polypeptide on its receptor.

28. The method of claim 27 wherein the antibody neutralizes the signal transduction.

29. The method of claim 26 wherein there is also an inhibition of chemokine release.

30. The method of claim 29, wherein the chemokine is GROα.

31. A method of reducing, inhibiting or preventing metastasis formation or tumor size comprising admixing an antibody with a polypeptide as shown in SEQ ID NO: 5, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

32. A method of inhibiting, reducing or preventing secretion of the polypeptide as shown by the amino acid sequence of SEQ ID NO: 2, comprising admixing an antibody with a polypeptide as shown in SEQ ID NO: 2, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

33. A method of inhibiting, reducing, or delaying progression of inflammation comprising admixing an antibody with a polypeptide as shown in SEQ ID NO: 2, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

34. A method of detecting a polypeptide comprising admixing the polypeptide with an antibody wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.

35. The method of claim 34, wherein the polypeptide comprising the amino acid sequence of SEQ ID NO: 2 or SEQ ID NO: 5, or a fragment thereof.

36. The method of claim 34 wherein the polypeptide is detected in serum.

37. The method of claim 36, wherein the serum is from a patient with cancer.

38. A method of inhibiting or reducing neutrophil infiltration comprising admixing an antibody with a polypeptide as shown in SEQ ID NO: 2, wherein the polypeptide is capable of binding the antibody produced by the hybridoma selected from:
a) the hybridoma of clone designation number 279.111.5.2 (ATCC Patent Deposit Designation PTA-6856);
b) the hybridoma of clone designation number 279.124.1.4 (ATCC Patent Deposit Designation PTA-6857);
c) the hybridoma of clone designation number 279.126.5.6.5 (ATCC Patent Deposit Designation PTA-6858); and
d) the hybridoma of clone designation number 279.121.7.4 (ATCC Patent Deposit Designation PTA-6859);
and
where in the antibody binds to the polypeptide.
